SEQUENCE LISTING (63 sequences)

SEQ ID NO: 1 - DNA, 10 bp, Artificial Sequence
  misc_feature: Primer
aatgctagcn

SEQ ID NO: 2 - DNA, 16 bp, Artificial Sequence
  misc_feature: Primer
atttctagaacttacn

SEQ ID NO: 3 - DNA, 28 bp, Artificial Sequence
  misc_feature: Primer
aattctagagtctgtccctaacatgccc

SEQ ID NO: 4 - DNA, 28 bp, Artificial Sequence
  misc_feature: Primer
aaaggtacctggaactgaggagcaggtg

SEQ ID NO: 5 - DNA, 24 bp, Artificial Sequence
  misc_feature: Primer
aatgctagcggctctcactccatg

SEQ ID NO: 6 - DNA, 27 bp, Artificial Sequence
  misc_feature: Primer
attgaattcttaggtgaggggcttggg

SEQ ID NO: 7 - protein, 10 aa, Artificial Sequence
  misc_feature: HLA Adapter
GGGGSGGGGS

SEQ ID NO: 8 - DNA, 42 bp, Artificial Sequence
  misc_feature: HLA Adapter
tttcagctgggggcggcggcggctctggcggcggcggctctg

SEQ ID NO: 9 - DNA, 42 bp, Artificial Sequence
  misc_feature: HLA Adapter
cagagccgccgccgccagagccgccgccgcccccagctgaaa

SEQ ID NO: 10 - DNA, 24 bp, Artificial Sequence
  misc_feature: Primer
aaagctagcggggacacccgacca

SEQ ID NO: 11 - DNA, 32 bp, Artificial Sequence
  misc_feature: Primer
aaagaattcattcatcttgctctgtgcagatt

SEQ ID NO: 12 - DNA, 27 bp, Artificial Sequence
  misc_feature: Primer
aattctagagaactgtggctgcaccat

SEQ ID NO: 13 - DNA, 29 bp, Artificial Sequence
  misc_feature: Primer
aaaggtaccacactctcccctgttgaagc

SEQ ID NO: 14 - DNA, 30 bp, Artificial Sequence
  misc_feature: Primer
aaagctagcatcaaagaagaacatgtgatc

SEQ ID NO: 15 - DNA, 36 bp, Artificial Sequence
  misc_feature: Primer
tttaagcttttagttctctgtagtctctgggagagg

SEQ ID NO: 16 - DNA, 32 bp, Artificial Sequence
  misc_feature: DRA Adapter 1
cggcggcggcggctctggcggcggcggctctg

SEQ ID NO: 17 - DNA, 40 bp, Artificial Sequence
  misc_feature: DRA Adapter 1
ctagcagagccgccgccgccagagccgccgccgccggtac

SEQ ID NO: 18 - protein, 60 aa, Artificial Sequence
  misc_feature: Spacer
GGGGSGGGGSGGGGSGGGGSGGGGSGGGGSGGGGSGGGGSGGGGSGGGGSGGGGSGGGGS

SEQ ID NO: 19 - protein, 30 aa, Artificial Sequence
  misc_feature: Spacer
GGGGSGGGGSGGGGSGGGGSGGGGSGGGGS

SEQ ID NO: 20 - DNA, 29 bp, Artificial Sequence
  misc_feature: Primer
aaaggtacccatcttgctctgtgcagatt

SEQ ID NO: 21 - DNA, 30 bp, Artificial Sequence
  misc_feature: Primer
aaaactagtatcaaagaagaacatgtgatc

SEQ ID NO: 22 - DNA, 36 bp, Artificial Sequence
  misc_feature: Primer
tttgaattcttagttctctgtagtctctgggagagg

SEQ ID NO: 23 - DNA, 29 bp, Artificial Sequence
  misc_feature: DRA 2
cggcggcggcggctctggcggcggcggca

SEQ ID NO: 24 - DNA, 37 bp, Artificial Sequence
  misc_feature: DRA 2
ctagtgccgccgccgccagagccgccgccgccggtac

SEQ ID NO: 25 - protein, 9 aa, Artificial Sequence
  misc_feature: Adapter
GGGGSGGGG

SEQ ID NO: 26 - DNA, 15 bp, Artificial Sequence
  misc_feature: Primer
ggggacacccgacca

SEQ ID NO: 27 - DNA, 18 bp, Artificial Sequence
  misc_feature: Primer
gactcgccgctgcactgt

SEQ ID NO: 28 - DNA, 21 bp, Artificial Sequence
  misc_feature: Primer
atcaaagaagaacatgtgatc

SEQ ID NO: 29 - DNA, 20 bp, Artificial Sequence
  misc_feature: Primer
ggtgatcggagtatagttgg

SEQ ID NO: 30 - DNA, 36 bp, Artificial Sequence
  misc_feature: Primer
gtgcagcggcgagtcatcaaagaagaacatgtgatc

SEQ ID NO: 31 - DNA, 24 bp, Artificial Sequence
  misc_feature: Primer
aaagctagcggggacacccgacca

SEQ ID NO: 32 - DNA, 32 bp, Artificial Sequence
  misc_feature: Primer
aaagaattcttaggtgatcggagtatagttgg

SEQ ID NO: 33 - DNA, 354 bp, Artificial Sequence
  misc_feature: C35
gccgcgatgagcggggagccggggcagacgtccgtagcgccccctcccgaggaggtcgagccgggcagtggggtccgcatcgtggtggagtactgtgaaccctgcggcttcgaggcgacctacctggagctggccagtgctgtgaaggagcagtatccgggcatcgagatcgagtcgcgcctcgggggcacaggtgcctttgagatagagataaatggacagctggtgttctccaagctggagaatgggggctttccctatgagaaagatctcattgaggccatccgaagagccagtaatggagaaaccctagaaaagatcaccaacagccgtcctccctgcgtcatcctgtga

SEQ ID NO: 34 - protein, 117 aa, Artificial Sequence
  misc_feature: C35
AAMSGEPGQTSVAPPPEEVEPGSGVRIVVEYCEPCGFEATYLELASAVKEQYPGIEIESRLGGTGAFEIEINGQLVFSKLENGGFPYEKDLIEAIRRASNGETLEKITNSRPPCVIL

SEQ ID NO: 35 - protein, 8 aa, Artificial Sequence
  misc_feature: ovalbumin 257-264 peptide
SIINFEKL

SEQ ID NO: 36 - DNA, 20 bp, Artificial Sequence
  misc_feature: Primer
cggcgagatgtctcacagga

SEQ ID NO: 37 - DNA, 20 bp, Artificial Sequence
  misc_feature: Primer
accccaccatctgcacaaag

SEQ ID NO: 38 - protein, 9 aa, Artificial Sequence
  misc_feature: C35 peptides
SVAPPPEEV

SEQ ID NO: 39 - protein, 8 aa, Artificial Sequence
  misc_feature: C35 peptides
VAPPPEEV

SEQ ID NO: 40 - protein, 8 aa, Artificial Sequence
  misc_feature: C35 peptides
EVEPGSGV

SEQ ID NO: 41 - protein, 10 aa, Artificial Sequence
  misc_feature: C35 peptides
EVEPGSGVRI

SEQ ID NO: 42 - protein, 8 aa, Artificial Sequence
  misc_feature: C35 peptides
EATYLELA

SEQ ID NO: 43 - protein, 9 aa, Artificial Sequence
  misc_feature: C35 peptides
ATYLELASA

SEQ ID NO: 44 - protein, 10 aa, Artificial Sequence
  misc_feature: C35 peptides
ATYLELASAV

SEQ ID NO: 45 - protein, 8 aa, Artificial Sequence
  misc_feature: C35 peptides
YLELASAV

SEQ ID NO: 46 - protein, 10 aa, Artificial Sequence
  misc_feature: C35 peptides
SAVKEQYPGI

SEQ ID NO: 47 - protein, 9 aa, Artificial Sequence
  misc_feature: C35 peptides
AVKEQYPGI

SEQ ID NO: 48 - protein, 8 aa, Artificial Sequence
  misc_feature: C35 peptides
GIEIESRL

SEQ ID NO: 49 - protein, 9 aa, Artificial Sequence
  misc_feature: C35 peptides
EIESRLGGT

SEQ ID NO: 50 - protein, 10 aa, Artificial Sequence
  misc_feature: C35 peptides
RLGGTGAFEI

SEQ ID NO: 51 - protein, 9 aa, Artificial Sequence
  misc_feature: C35 peptides
GTGAFEIEI

SEQ ID NO: 52 - protein, 8 aa, Artificial Sequence
  misc_feature: C35 peptides
EIEINGQL

SEQ ID NO: 53 - protein, 9 aa, Artificial Sequence
  misc_feature: C35 peptides
EIEINGQLV

SEQ ID NO: 54 - protein, 9 aa, Artificial Sequence
  misc_feature: C35 peptides
DLIEAIRRA

SEQ ID NO: 55 - protein, 8 aa, Artificial Sequence
  misc_feature: C35 peptides
LIEAIRRA

SEQ ID NO: 56 - protein, 10 aa, Artificial Sequence
  misc_feature: C35 peptides
AIRRASNGET

SEQ ID NO: 57 - protein, 8 aa, Artificial Sequence
  misc_feature: C35 peptides
RASNGETL

SEQ ID NO: 58 - protein, 10 aa, Artificial Sequence
  misc_feature: C35 peptides
KITNSRPPCV

SEQ ID NO: 59 - protein, 9 aa, Artificial Sequence
  misc_feature: C35 peptides
ITNSRPPCV

SEQ ID NO: 60 - protein, 10 aa, Artificial Sequence
  misc_feature: C35 peptides
ITNSRPPCVI

SEQ ID NO: 61 - protein, 9 aa, Artificial Sequence
  misc_feature: C35 peptides
EVEPGSGVR

SEQ ID NO: 62 - protein, 9 aa, Artificial Sequence
  misc_feature: C35 peptides
EPCGFEATY

SEQ ID NO: 63 - protein, 9 aa, Artificial Sequence
  misc_feature: C35 peptides
ASNGETLEK

(19) United States
(12) Patent Application Publication
(10) Pub. No.: US 2003/0166277 A1
(43) Pub. Date: Sep. 4, 2003

(54) Targeted vaccine delivery systems

(75) Inventors: Maurice Zauderer, Pittsford, NY (US); Ernest S. Smith, Rochester, NY (US)

Correspondence Address: STERNE, KESSLER, GOLDSTEIN & FOX PLLC, 1100 NEW YORK AVENUE, N.W., WASHINGTON, DC, 20005, US

(73) Assignee: University of Rochester

(21) Appl. No.: 09/833,203

(22) Filed: Apr. 12, 2001

Related U.S. Application Data
(60) Provisional application No. 60/196,472, filed on Apr. 12, 2000.

(51) Int. Cl. A61K 39/395; C12P 21/08; C07K 16/46

(52) U.S. Cl. 435/372; 530/391.1; 424/178.1

(57) ABSTRACT

The present invention is directed to a novel targeted vaccine delivery system, comprising one or more MHC-peptide complexes linked to an antibody which is specific for a cell surface marker. The complexes of the invention are useful for treating and/or preventing cancer, infectious diseases, autoimmune diseases, and/or allergies.

### CROSS-REFERENCE TO RELATED APPLICATIONS

This application claims priority benefit of U.S. Appl. No. 60/196,472, filed Apr. 12, 2000, which is incorporated by reference herein.

### STATEMENT REGARDING FEDERALLY-SPONSORED RESEARCH AND DEVELOPMENT

Not applicable.

### BACKGROUND OF THE INVENTION

1. Field of the Invention

The present invention relates to immunology. More specifically, the present invention relates to vaccines and methods for modifying immune responses.

2. Background Art

T lymphocytes are both key effector cells and key regulatory cells of the immune system. The ability to stimulate or inhibit specific T cell responses is a major goal for the immunotherapy of cancer, infectious diseases, and autoimmune diseases. T cell specificity is mediated by a T cell receptor (TCR) on the surface of the T cells. Each TCR is specific for a complex of a unique peptide epitope of a protein antigen associated with a major histocompatibility complex (MHC) molecule on the surface of a cell. There are two classes of MHC proteins which bind to TCRs in conjunction with peptide antigens: MHC class I proteins, which are found on the membranes of all nucleated cells; and MHC class II proteins, which are found only on certain cells of the immune system. The two major classes of T cells, CD8+ and CD4+, are selected to be specific for peptide epitopes that associate, respectively, with MHC class I and class II molecules on the antigen presenting cell. Polymorphism within each class of MHC molecule determines which peptide fragments bind with functional affinity to the MHC molecules expressed by a particular individual.

Peptide-MHC complexes have a relatively fast dissociation rate from the TCR. Multimeric peptide-MHC complexes have, as expected, been shown to have slower dissociation rates and are far more suitable than soluble monomeric complex for binding to receptors on a specific T cell. A technology for engineering tetrameric peptide-MHC complexes based on addition of biotin to the COOH-terminus of the MHC class I heavy chain and high affinity association with tetrameric avidin has been developed (Altman, J. D., et al., Science 274:94-96 (1996)). A similar strategy has been adapted for MHC class II molecules (Schmitt, L. et al., Proc. Natl. Acad. Sci., USA. 96:6581-6586 (1999); Zarutskie, J. A. et al., Biochemistry 38:5878-5887 (1999)). Such molecules are referred to as peptide-MHC tetramers and are widely employed for staining of specific T cells. A different form of dimeric peptide-MHC complex has been shown to activate specific T cells in vitro (Hamad, A. R. A. et al., J. Exp. Med. 188:1633-1640 (1998)).

Binding of peptide-MHC complexes to T cells is, in general, not sufficient to induce T cell proliferation and differentiation. Additional costimulatory signals delivered through interactions between other membrane molecules of the T cell and the antigen presenting cell are required for optimal T cell activation. Indeed, signaling through T cell antigen receptor alone in the absence of costimulation can result in tolerization rather than activation.

Dendritic cells are a uniquely potent lineage of professional antigen presenting cell that express high membrane levels of both MHC and co-stimulatory molecules. A number of vaccine strategies target antigen presentation by dendritic cells through ex vivo introduction of antigen into dendritic cells or provision of GM-CSF and/or other cytokines together with a source of antigen in vivo in order to promote recruitment and maturation of dendritic cells at the site of antigen deposit. Ex vivo strategies require complex manipulations of patient materials which are time consuming and expensive. In vivo manipulations are limited by the efficiency with which dendritic cells are recruited and with which they take up, process, and present antigenic peptide to specific T cells.

Both T cells and activated dendritic cells express membrane differentiation antigens that can be targeted by specific antibodies. Some of the corresponding membrane molecules may deliver either positive or negative activation signals to the T cell or dendritic cell precursor. These include the T cell markers CD28 and CTLA-4 (CD 152) which are, respectively, thought to mediate positive and negative co-stimulator interactions. In contrast, the dendritic cell differentiation markers CD83, CMRF-44 and CMRF-56 are not known to have a specific function in membrane signaling. CD83, in particular, has been tested in a variety of experiments and never found to have an effect beyond target cell recognition.

Methods are available to target a specific ligand or regulatory molecule to an antigen positive cell by genetically linking the specificity domain of an antibody specific for that antigen to a particular ligand or cytokine. Fusion proteins encoded in this fashion may retain both antigen specificity and ligand or cytokine function. Examples of such reagents have been described in which the ligand coding sequence is linked to either the carboxyl or amino terminus of an antibody chain which may itself be either whole or truncated (Morrison, S. L. et al., Clin. Chem. 34:1668-1675 (1988); Shin, S. U. and Morrison, S. L., Meth. in Enzymol. 178:459-476(1989); Porto, J. D. et al., Proc. Nat'l. Acad. Sci. USA 90:6671-6675 (1993); Shin, S.-U. et al, J. Immunol 158:4797-4804 (1997)). A particularly flexible construct has been described, in which an avidin molecule is linked to the carboxyl-terminus of the heavy chain of an antibody that can target the transferrin receptor and can, in principle, deliver any biotinylated ligand to the target cell (Penichet, M. L. et al., J. Immunol. 163:4421-4426 (1993)).

The key requirements for construction of a delivery system that can target specific cells and tissues to deliver a ligand or cytokine are to identify an appropriate target molecule, select an antibody with a specificity domain with high affinity for that target molecule, and to link an effective concentration of ligand or cytokine to that antibody specificity domain. For the specific purpose of vaccine delivery, the relevant ligand is a specific peptide-MHC complex, preferably in multimeric form. Two types of constructs would be especially useful: 1) a delivery vehicle that could target professional antigen presenting cells, such as dendritic cells, or other cells, such as tumor cells, epithelial cells or fibroblasts, and deliver an effective concentration of peptide-MHC complex to modulate (i.e., stimulate or inhibit) a specific T cell response; and 2) a delivery vehicle that could target T cells through either positive or negative regulatory molecules, CD28 and CTLA-4, or lymphokine receptor, CD25, on the T cell and simultaneously deliver an effective concentration of peptide-MHC complex to signal through the specific TCR.

In view of the diversity of antigens expressed in cancer and in infectious or autoimmune disease, and the natural polymorphism of human MHC, effective use of such fusion proteins for immunotherapy would be greatly facilitated by the ability to flexibly couple different multimeric peptide-MHC complexes to one or more dendritic cell or T cell targeting specificities.

### BRIEF SUMMARY OF THE INVENTION

The present invention provides compounds useful for modulating, i.e., either inhibiting or stimulating, an immune response. The compound of the invention comprises one or more MHC-peptide complexes linked to an antibody or fragment thereof specific for a cell surface marker.

In one embodiment, the compound comprises one or more MHC-peptide complexes linked to an antibody or fragment thereof specific for a cell surface marker. In one embodiment, the MHC-peptide complexes comprise an MHC class I α chain or fragment thereof, a β₂-microglobulin molecule or fragment thereof, and an antigenic peptide bound in the MHC groove. In another embodiment, the MHC-peptide complexes comprise an MHC class II α chain or fragment thereof, an MHC class II β chain or fragment thereof, and an antigenic peptide bound in the MHC groove. Preferably, the MHC-peptide complexes are linked to the carboxyl terminus of the antibody or fragment thereof.

In another embodiment, the compound comprises two or more MHC-peptide complexes and an antibody or fragment thereof specific for a cell surface marker, wherein the MHC-peptide complexes and the antibody are linked to a multivalent compound. In one embodiment, the MHC-peptide complexes comprise an MHC class I α chain or fragment thereof, β₂-microglobulin or fragment thereof, and an antigenic peptide bound in the MHC groove. In another embodiment, the MHC-peptide complexes comprise an MHC class II α chain or fragment thereof, an MHC class II β chain or fragment thereof, and an antigenic peptide bound in the MHC groove. The MHC-peptide complexes may be linked to the antibody through the multivalent compound.

In certain embodiments, the antibody is specific for a cell surface marker of a professional antigen presenting cell, more particularly a dendritic cell. In other embodiments, the antibody is specific for a cell surface marker of a tumor cell, an epithelial cell or a fibroblast. In other embodiments, the antibody is specific for a cell surface marker of a T cell.

In certain embodiments, the antigenic peptide is derived from a cancer cell. In other embodiments, the antigenic peptide is derived from an infectious agent or an infected cell. In still other embodiments, the antigenic peptide is derived from an allergen or the target tissue of an autoimmune disease. In other embodiments, the antigenic peptide is synthetic.

Also provided are method of modulating, i.e., either stimulating or inhibiting, and immune response, comprising administering to an animal an effective amount of a compound or composition of the invention.

### BRIEF DESCRIPTION OF THE DRAWINGS/FIGURES

FIG. 1 shows the structure of Antibody-Avidin fusion protein with bound biotinylated MHC class I molecules.

FIG. 2 shows the structure of Antibody-Avidin fusion protein with bound biotinylated MHC class II molecules.

FIG. 3 shows the structure of Antibody-MHC class I fusion proteins.

FIG. 4 shows the structure of Antibody-MHC class II fusion proteins.

FIG. 5 shows the structure of Antibody-Single Chain MHC Class II fusion molecules.

FIG. 6 shows the structure of Antibody-Two Domain MHC class II fusion molecules.

FIG. 7 shows the nucleotide (SEQ ID NO:33) and amino acid (SEQ ID NO:34) sequence of C35.

### DETAILED DESCRIPTION OF THE INVENTION

The present invention provides compounds which are useful for modulating, i.e., either inhibiting or stimulating, an immune response. The compounds comprise one or more MHC-peptide complexes linked to an antibody or fragment thereof specific for a cell surface marker. The compounds are useful for stimulating desirable immune responses, for example, immune responses against infectious agents or cancer; or for inhibiting undesirable immune responses, such as allergic responses, allograft rejections, and autoimmune diseases. The present invention targets a peptide-MHC complex to professional antigen presenting cells, such as dendritic cells, B cells, or macrophages; tumor cells; epithelial cells; fibroblasts; T cells; or other cells, by linking one or more peptide-MHC complexes to an antibody or fragment thereof specific for a surface antigen of the targeted cell type. Depending on the targeted cell type, this will lead to either very efficient stimulation or inhibition of antigen specific T cell activity.

In certain embodiments, the compound comprises one or more MHC-peptide complexes linked to an antibody or fragment thereof, wherein the antibody is specific for a cell surface marker. In one embodiment, the MHC-peptide complex comprises an MHC class I α chain or fragment thereof, a β₂-microglobulin molecule or fragment thereof, and an antigenic peptide bound in the MHC groove. In certain embodiments, the MHC class I α chain is linked to the heavy chain of the antibody, and the β₂-microglobulin molecule is linked to the light chain of the antibody; the MHC class I α chain is linked to the light chain of the antibody, and the β₂-microglobulin molecule is linked to the heavy chain of the antibody; the MHC class I α chain is linked to the heavy chain of the antibody; the MHC class I α chain is linked to the light chain of the antibody; the β₂-microglobulin molecule is linked to the heavy chain of the antibody; or the β₂-microglobulin molecule is linked to the light chain of the antibody.

Alternatively, the MHC-peptide complex comprises an MHC class II α chain or fragment thereof, an MHC class II β chain or fragment thereof, and an antigenic peptide bound in the MHC groove. In certain embodiments, the MHC class II α chain is linked to the heavy chain of the antibody, and the MHC class II β chain is linked to the light chain of the antibody; the MHC class II α chain is linked to the light chain of the antibody, and the MHC class II β chain is linked to the heavy chain of the antibody; the MHC class II α chain is linked to the heavy chain of the antibody; the MHC class II α chain is linked to the light chain of the antibody; the MHC class II β chain is linked to the heavy chain of the antibody; or the MHC class II β chain is linked to the light chain of the antibody.

The MHC-peptide complexes may be linked to the either the carboxyl or amino terminus of the antibody, or they may be linked to the antibody at a site other than the carboxyl or amino termini. Preferably, the MHC-peptide complexes are linked to the carboxyl terminus of the antibody.

Preferably, there are two MHC-peptide complexes per antibody. The attachment of the MHC chains to the antibody chains may be direct, i.e., without any intermediate sequence, or through a linker amino acid sequence, a linker molecule, or a chemical bond. For example, the MHC-peptide complex is linked through its a chain to a monovalent Fab fragment of an antibody. This type of construct is, for example, of particular benefit for targeting CD154, the CD40 ligand expressed on T cells whose interaction with CD40 serves to activate antigen presenting cells. The crosslinking activity of a multivalent antibody may by itself induce broad and deleterious non-specific inflammatory responses. By coupling monomeric anti-CD 154 to one or more peptide-MHC complexes it may be possible to elicit a more focused antigen-specific response.

In another embodiment, the compound comprises two or more MHC-peptide complexes, an antibody or fragment thereof which binds to a cell surface marker, and a multivalent compound. In certain embodiments, the MHC-peptide complexes comprises an MHC class I α chain or fragment thereof, β₂-microglobulin or fragment thereof, and an antigenic peptide bound in the MHC groove. In certain other embodiments, the MHC-peptide complex comprises an MHC class II α chain or fragment thereof, an MHC class II β chain or fragment thereof, and an antigenic peptide bound in the MHC groove.

In further embodiments, the compound comprises two or more MHC-peptide complexes and a multivalent compound. The MHC-peptide complexes may comprise an MHC class I α chain or fragment thereof, β₂-microglobulin or fragment thereof, and an antigenic peptide bound in the MHC groove; or an MHC class II α chain or fragment thereof, an MHC class II β chain or fragment thereof, and an antigenic peptide bound in the MHC groove. Such compounds are useful for modulating an immune response and for administration as vaccines.

The MHC-peptide complexes may be linked to the multivalent compound through any site. For example, the MHC-peptide complexes may be linked through the MHC class I α chain, the β₂-microglobulin molecule, the MHC class II α chain, and/or the MHC class II β chain.

The compound of the invention may further comprise a cytokine or lymphokine. The cytokine or lymphokine may be linked to the multivalent compound, the antibody, or the MHC-peptide complex. For example, the multivalent compound may be avidin or streptavidin, and the cytokine or lymphokine may be biotinylated. Alternatively, the cytokine or lymphokine may be directly fused to the antibody or MHC-peptide complex.

Cytokines or lymphokines useful in the present invention include, but are not limited to, interleukins (e.g., IL-2, IL-3, IL-4, IL-5, IL-6, IL-10, IL-12, IL-15, and IL-18), α interferons (e.g., IFNα), β interferons (e.g., IFNβ), γ interferons (e.g., IFNγ), granulocyte-macrophage colony stimulating factor (GM-CSF), and transforming growth factor (TGF, e.g., TGFα and TGFβ).

The compound of the invention may further comprise other therapeutic agents. The therapeutic agent or agents may be linked to the multivalent compound, the antibody, or the MHC-peptide complex. Examples of therapeutic agents include, but are not limited to, antimetabolites, alkylating agents, anthracyclines, antibiotics, and anti-mitotic agents. Antimetabolites include methotrexate, 6-mercaptopurine, 6-thioguanine, cytarabine, 5-fluorouracil decarbazine. Alkylating agents include mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin. Anthracyclines include daunorubicin (formerly daunomycin) and doxorubicin (also referred to herein as adriamycin). Additional examples include mitozantrone and bisantrene. Antibiotics include dactinomycin (formerly actinomycin), bleomycin, mithramycin, and anthramycin (AMC). Antimytotic agents include vincristine and vinblastine (which are commonly referred to as vinca alkaloids). Other cytotoxic agents include procarbazine, hydroxyurea, asparaginase, corticosteroids, mytotane (O,P′-(DDD)), interferons. Further examples of cytotoxic agents include, but are not limited to, ricin, doxorubicin, taxol, cytochalasin B, gramicidin D, ethidium bromide, etoposide, tenoposide, colchicin, dihydroxy anthracin dione, 1-dehydrotestosterone, and glucocorticoid.

Clearly analogs and homologs of such therapeutic and cytotoxic agents are encompassed by the present invention. For example, the chemotherapuetic agent aminopterin has a correlative improved analog namely methotrexate. Further, the improved analog of doxorubicin is an Fe-chelate. Also, the improved analog for 1-methylnitrosourea is lomustine. Further, the improved analog of vinblastine is vincristine. Also, the improved analog of mechlorethamine is cyclophosphamide.

The compound of the invention may be labeled, so as to be directly detectable, or will be used in conjunction with secondary labeled immunoreagents which will specifically bind the compound. In general, the label will have a light detectable characteristic. Preferred labels are fluorophors, such as fluorescein isothiocyanate (FITC), rhodamine, Texas Red, phycoerythrin and allophycocyanin. Other labels of interest may include dyes, enzymes, chemiluminescers, particles, radioisotopes, or other directly or indirectly detectable agent. Alternatively, a second stage label may be used, e.g. labeled antibody directed to one of the constituents of the compound of the invention.

MHC class I molecules consist of an a (heavy) chain, coded for by MHC genes, associated with β₂-microglobulin, coded for by non-MHC genes. The β₂-microglobulin protein and α₃ segment of the heavy chain are associated; the α₁ and α₂ regions of the heavy chain form the base of the antigen-binding pocket (Science 238:613-614(1987); Bjorkman, P. J. et al., Nature 329:506-518 (1987)). An α chain may come from genes in the A, B or C subgroup. Class I molecules bind peptides of about 8-9 amino acids in length. All humans have between three and six different class I molecules, which can each bind many different types of peptides.

MHC class II molecules are coded entirely by MHC genes and consist of two similar polypeptide chains each about 30 kD, again one called α the other β. The chains may come from the DP, DQ, or DR gene groups. There about 40 known different human MHC class II molecules. All have the same basic structure but vary subtly in their molecular structure. MHC class II molecules bind peptides of 13-18 amino acids in length.

The term “MHC” encompasses similar molecules in different species. In mice, the MHC is termed H-2, in humans it is termed HLA for “Human Leucocyte Antigen”. When used herein, “MHC” is universally applied to all species.

Conventional identifications of particular MHC variants are used herein. For example, HLA-B 17 refers to a human leucocyte antigen from the B gene group (hence a class I type MHC) gene position (known as a gene locus) number 17; gene HLA-DR11, refers to a human leucocyte antigen coded by a gene from the DR region (hence a class II type MHC) locus number 11.

MHC molecules useful in the present invention include, but are not limited to, HLA specificities such as A (e.g. A1-A74), B (e.g., B 1-B77), C (e.g., C1-C11), D (e.g., D1-D26), DR (e.g., DR1-DR8), DQ (e.g., DQ1-DQ9) and DP (e.g. DP1-DP6). More preferably, HLA specificities include A1, A2, A3, A11, A23, A24, A28, A30, A33, B7, B8, B35, B44, B53, B60, B62, DR1, DR2, DR3, DR4, DR7, DR8, and DR 11. It is possible to tissue type a person by serological or genetic analysis to define which MHC class I or II molecule variants each person has using methods known in the art.

In a preferred embodiment, the MHC protein subunits are a soluble form of the normally membrane-bound protein. The soluble form is derived from the native form by deletion of the transmembrane domain. The MHC molecules may also be truncated by removal of both the cytoplasmic and transmembrane domains. The protein may be truncated by proteolytic cleavage, or by expressing a genetically engineered truncated form.

For class I proteins, the soluble form will include the α1, α2 and α3 domain. Not more than about 10, usually not more than about 5, preferably none of the amino acids of the transmembrane domain will be included. The deletion may extend as much as about 10 amino acids into the α3 domain, preferably none of the amino acids of the α3 domain will be deleted. The deletion will be such that it does not interfere with the ability of the α3 domain to fold into a disulfide bonded structure. The class I β chain, β₂-microglobulin, lacks a transmembrane domain in its native form, and need not be truncated. However, fragments of β₂-microglobulin are useful in the present invention.

Soluble class II subunits will include the α1 and α2 domains for the a subunit, and the β₁ and β₂ domains for the P subunit. Not more than about 10, usually not more than about 5, preferably none of the amino acids of the transmembrane domain will be included. The deletion may extend as much as about 10 amino acids into the α2 or β2 domain, preferably none of the amino acids of the α2 or β2 domain will be deleted. The deletion will be such that it does not interfere with the ability of the α2 or β2 domain to fold into a disulfide bonded structure.

One may wish to introduce a small number of amino acids at the polypeptide termini, usually not more than 20, more usually not more than 15. The deletion or insertion of amino acids will usually be as a result of the needs of the construction, providing for convenient restriction sites, addition of processing signals, ease of manipulation, improvement in levels of expression, or the like. In addition, one may wish to substitute one or more amino acids with a different amino acid for similar reasons, usually not substituting more than about five amino acids in any one domain.

The α and β subunits may be separately produced and allowed to associate to form a stable heteroduplex complex (see Altman et al. (1993), or Garboczi et al. (1992)), or both of the subunits may be expressed in a single cell. An alternative strategy is to engineer a single molecule having both the α and β subunits. A “single-chain heterodimer” is created by fusing together the two subunits using a short peptide linker, e.g. a 15 to 25 amino acid peptide or linker. (Burrows G. G. et al, J. Immunology 161: 5987-5996 (1998)). Zhu, X. et al., Eur. J. Immunol. 27: 1933-1941 (1997) have also described production of a single chain class II molecule by fusion of coding sequences for the class II subunits including α1 and α2 and β1 and β2. See Bedzyk et al, J. Biol. Chem. 265:18615 (1990) for similar structures with antibody heterodimers. The soluble heterodimer may also be produced by isolation of a native heterodimer and cleavage with a protease, e.g. papain, to produce a soluble product.

The MHC molecules useful in the present invention may be from any mammalian or avian species, for example, primates (esp. humans), rodents, rabbits, equines, bovines, canines, felines, etc.

MHC molecules useful in the compounds of the present invention may be isolated from a multiplicity of cells, e.g., transformed cell lines JY, BM92, WIN, MOC, and MG, using a variety of techniques including solubilization by treatment with papain, by treatment with 3M KCl, and by treatment with detergent. In a preferred method, detergent extraction of Class II protein from lymphocytes followed by affinity purification is used. Detergent can then be removed by dialysis or selection binding beads, e.g., Bio Beads.

Methods for purifying the murine I-A (Class II) histocompatibility proteins have been disclosed by Turkewitz, A. P. et al., Mol. Immunol. 20:1139-1147 (1983). Isolation of these detergent-soluble HLA antigens was described by Springer, T. A. et al., Proc Natl Acad Sci USA 73:2481-2485 (1976). Soluble HLA-A2 can be purified after papain digestion of plasma membranes from the homozygous human lymphoblastoid cell line J-Y as described by Turner, M. J. et al, J. Biol. Chem. 250:4512-4519 (1975); Parham P. et al., J. Biol. Chem. 252:7555-7567 (1977). Papain cleaves the 44 kd chain close to the transmembrane region yielding a molecule comprised of α_{1, α}₂, α₃ and β₂-micro globulin.

Alternatively, the amino acid sequence of a number of MHC proteins are known, and the genes have been cloned, therefore, the proteins can be made using recombinant methods. For example, the heavy (α) and light (β) chains of an MHC class II molecule, or the a chain of an MHC class I molecule, are synthesized using a truncation of the carboxyl terminus coding sequence which effects the deletion of the hydrophobic domain, and the carboxyl termini coding sequence can be arbitrarily chosen to facilitate the conjugation of the antibody or binding intermediate. The coding sequence for the α and β chains are then inserted into expression vectors, expressed separately in an appropriate host, such as E. coli, yeast, insect cells, or other suitable cells, and the recombinant proteins obtained are recombined in the presence of the peptide antigen and, in the case of MHC class I, β₂-microglobulin. Known, partial and putative HLA amino acid and nucleotide sequences, including the consensus sequence, are published (see, e.g., Zemmour and Parham, Immunogenetics 33:310-320 (1991)), and cell lines expressing HLA variants are known and generally available as well, many from the American Type Culture Collection (“ATCC”).

As the availability of the gene permits ready manipulation of the sequence, a construct can be made which includes hybrid Class I and Class II features, wherein the α₁ and β₁ domains of MHC class II are linked through a flexible portion that permits intramolecular dimerization between these domains resulting in an edge-to-edge β sheet contact. This two domain class II molecule can be employed directly or as a fusion with the α₃ domain of Class I with β₂-microglobulin coexpressed to stabilize the complex. Construction of expression vectors and recombinant production from the appropriate DNA sequences are performed by methods known in the art.

Antigenic peptides useful within the present invention include any peptide which is capable of modulating an immune response in an animal when presented in conjunction with an MHC molecule. Peptides may be derived from foreign antigens or from autoantigens.

The antigenic peptide will be from about 6 to 12 amino acids in length for complexes with MHC class I proteins, usually from about 8 to 10 amino acids, most preferably 8 or 9 amino acids. The peptide will be from about 6 to 20 amino acids in length for complexes with MHC class II proteins, preferably from about 10 to 18 amino acids, more preferably 15, 16, 17, or 18 amino acids.

Methods for determining whether a particular peptide will bind to a particular MHC molecule are known in the art. See, for example, Parker et al., J. Immunol. 149:3580-3587 (1992); Southwood et al., J. Immunol. 160:3363-3373 (1998); Stumiolo et al., Nature Biotechnol. 17:5555-560 (1999).

Peptides may be loaded onto MHC via various means. Preferably, for MHC molecules that are produced recombinantly, peptides with low affinity for MHC are added to the culture medium, to ensure proper folding of MHC. The MHC molecules are then solubilized with enzymes such as papain or pepsin. The antigenic peptides are then added to the MHC molecules in solution and displace the low affinity peptides.

The peptides may be loaded onto the MHC molecules in various forms. For example, a homogenous population of a known antigenic peptide may be added to the MHC in solution. Alternatively, a protein may be degraded chemically or enzymatically, for example, and added to the MHC molecules in this form. For example, a protein of interest is degraded with chymotrypsin and the resultant mixture of peptide “fragments” is added to the MHC molecules; the MHC are then allowed to “choose” the appropriate peptides to load onto the MHC molecules. Alternatively, mixtures of peptides from different proteins may be added to the MHC. For example, extracts from tumor cells or infected cells may be added to the MHC molecules in solution.

Peptides according to the present invention may be obtained from naturally-occurring sources or may be synthesized using known methods. For example, peptides may be synthesized on an Applied Biosystems synthesizer, ABI 431A (Foster City, Calif.) and subsequently purified by HPLC. Alternatively, DNA sequences can be prepared which encode the particular peptide and may be cloned and expressed to provide the desired peptide. In this instance a methionine may be the first amino acid. In addition, peptides may be produced by recombinant methods as a fusion to proteins that are one of a specific binding pair, allowing purification of the fusion protein by means of affinity reagents, followed by proteolytic cleavage, usually at an engineered site to yield the desired peptide (see for example Driscoll et al., J. Mol. Bio. 232:342-350 (1993)). The peptides may also be isolated from natural sources and purified by known techniques, including, for example, chromatography on ion exchange materials, separation by size, immunoaffinity chromatography and electrophoresis.

Isolation or synthesis of “random” peptides may also be appropriate, particularly when one is attempting to ascertain a particular epitope in order to load an empty MHC molecule with a peptide most likely to stimulate T cells. One may produce a mixture of “random” peptides via use of proteasomes or by subjecting a protein or polypeptide to a degradative process—e.g., digestion with chymotrypsin—or peptides may be synthesized.

If one is synthesizing peptides, e.g., random 8-, 9- and 18-amino acid peptides, all varieties of amino acids are preferably incorporated during each cycle of the synthesis. It should be noted, however, that various parameters—e.g., solvent incompatibility of certain amino acids—may result in a mixture which contains peptides lacking certain amino acids. The process should thus be adjusted as needed—i.e., by altering solvents and reaction conditions—to produce the greatest variety of peptides.

In one embodiment, the antigenic peptide is derived from a cancerous cell, or promotes an immune response against a cancerous cell. In one embodiment, the antigenic peptide is derived from C35 (SEQ ID NOs:33 and 34).

A number of computer algorithms have been described for identification of peptides in a larger protein that may satisfy the requirements of peptide binding motifs for specific MHC class I or MHC class II molecules. Because of the extensive polymorphism of MHC molecules, different peptides will often bind to different MHC molecules. Table 1 lists C35 peptides predicted for binding to the HLA class I molecule HLA-A*0201 as well as a few limited examples of C35 peptides that express binding motifs specific for other selected class I MHC molecules. Table 2 lists four C35 peptides identified as likely candidates for binding to a variety of HLA class II molecules. These peptides are, in general, longer than those binding to HLA class I and more degenerate in terms of binding to multiple HLA class II molecules. Other C35 peptides which bind to specific HLA molecules are predicted in U.S. Application Ser. No. ______, filed Apr. 4, 2001 (Attorney Docket Ser. No. 1821.0040001), the disclosure of which is incorporated by reference herein.

TABLE 1
Predicted C35 HLA Class I epitopes*

| HLA restriction | Inclusive | |
|---|---|---|
| element | amino acids | Sequence |
| A*0201 | 9-17 | SVAPPPEEV (SEQ ID NO:38) |
| A*0201 | 10-17 | VAPPPEEV (SEQ ID NO:39) |
| A*0201 | 16-23 | EVEPGSGV (SEQ ID NO:40) |
| A*0201 | 16-25 | EVEPGSGVRI (SEQ ID NO:41) |
| A*0201 | 36-43 | EATYLELA (SEQ ID NO:42) |
| A*0201 | 37-45 | ATYLELASA (SEQ ID NO:43) |
| A*0201 | 37-46 | ATYLELASAV (SEQ ID NO:44) |
| A*0201 | 39-46 | YLELASAV (SEQ ID NO:45) |
| A*0201 | 44-53 | SAVKEQYPGI (SEQ ID NO:(46) |
| A*0201 | 45-53 | AVKEQYPGI (SEQ ID NO:47) |
| A*0201 | 52-59 | GIEIESRL (SEQ ID NO:48) |
| A*0201 | 54-62 | EIESRLGGT (SEQ ID NO:49) |
| A*0201 | 58-67 | RLGGTGAFEI (SEQ ID NO:50) |
| A*0201 | 61-69 | GTGAFEIEI (SEQ ID NO:51) |
| A*0201 | 66-73 | EIEINGQL (SEQ ID NO:52) |
| A*0201 | 66-74 | EIEINGQLV (SEQ ID NO:53) |
| A*0201 | 88-96 | DLIEAIRRA (SEQ ID NO:54) |
| A*0201 | 89-96 | LIEAIRRA (SEQ ID NO:55) |
| A*0201 | 92-101 | AIRRASNGET (SEQ ID NO:56) |
| A*0201 | 95-102 | RASNGETL (SEQ ID NO:57) |
| A*0201 | 104-113 | KITNSRPPCV (SEQ ID NO:58) |
| A*0201 | 105-113 | ITNSRPPCV (SEQ ID NO:59) |
| A*0201 | 105-114 | ITNSRPPCVI (SEQ ID NO:60) |
| A*3101 | 16-24 | EVEPGSGVR (SEQ ID NO:61) |
| B*3501 | 30-38 | EPCGFEATY (SEQ ID NO:62) |
| A*30101 | 96-104 | ASNGETLEK (SEQ ID NO:63) |
| supermotif | | |

*predicted using rules found at the SYFPEITHI website (wysiwyg://35/http://134.2.96.221/scripts/hlaserver.dll/EpPredict.htm) and are based on the book “MHC Ligands and Peptide Motifs” by Rammensee, H. G., Bachmann, J. and S. Stevanovic. Chapman & Hall, New York, 1997.

TABLE 2
Predicted C35 HLA class II epitopes*

| | In- | |
|---|---|---|
| | clusive | |
| | amino | |
| Sequence | acids | Restriction elements |
| SGVRIVVEYCEPC | 21-35 | DRB1*0101, DRB1*0102, DRB1*0301, |
| GF (SEQ ID | | DRB1*0401, DRB1*0404, DRB1*0405, |
| NO:62) | | DRB1*0410, DRB1*0421, DRB1*0701, |
| | | DRB1*0801, DRB1*0804, DRB1*0806, |
| | | DRB1*1101, DRB1*1104, DRB1*1106, |
| | | DRB1*1107, DRB1*1305, DRB1*1307, |
| | | DRB1*1321, DRB1*1501, DRB1*1502, |
| | | DRB5*0101 |
| SRLGGTGAFEIEI | 57-75 | DRB1*0101, DRB1*0102, DRB1*0301, |
| NGQLVF (SEQ ID | | DRB1*0401, DRB1*0402, DRB1*0421, |
| NO:63) | | DRB1*0701, DRB1*0804, DRB1*0806, |
| | | DRB1*1101, DRB1*1104, DRB1*1106, |
| | | DRB1*1305, DRB1*1321, DRB1*1501, |
| | | DRB1*1502, DRB5*0101 |
| GAFEIEINGQLVF | 63-83 | DRB1*0101, DRB1*0102, DRB1*0301, |
| SKLENGGF (SEQ | | DRB1*0401, DRB1*0402, DRB1*0404, |
| ID NO:64) | | DRB1*0405, DRB1*0410, DRB1*0421, |
| | | DRB1*0701, DRB1*0804, DRB1*0806, |
| | | DRB1*1101, DRB1*1104, DRB1*1106, |
| | | DRB1*1107, DRB1*1305, DRB1*1307, |
| | | DRB1*1311, DRB1*1321, DRB1*1501, |
| | | DRB1*1502, DRB5*0101 |
| FPYEKDLIEAIRR | 83-103 | DRB1*0101, DRB1*0102, DRB1*0301, |
| ASNGETLE (SEQ | | DRB1*0401, DRB1*0402, DRB1*0404, |
| ID NO:65) | | DRB1*0405, DRB1*0410, DRB1*0421, |
| | | DRB1*0701, DRB1*0801, DRB1*0802, |
| | | DRB1*0804, DRB1*0806, DRB1*1101, |
| | | DRB1*1104, DRB1*1106, DRB1*1107, |
| | | DRB1*1305, DRB1*1307, DRB1*1311, |
| | | DRB1*1321, DRB1*1501, DRB1*1502, |
| | | DRB5*0101 |

*Class II MHC epitopes predicted using TEPITOPE software. Sturniolo, T., et al., Nature Biotechnol. 17:555-571 (1999)

Non-limiting examples of other peptides derived from cancer cells are described in Table 3.

TABLE 3
Peptides derived from cancer cells

| Peptide Antigen(s) | Expressed in | MHC | HLA allele | Ref. |
|---|---|---|---|---|
| Melan A/MART-1 (26-35) | Melanoma | I | A*0201, | 1-3 |
| Melan A/MART-1 (51-73) | Melanoma | II | DRB1*0401 | 4 |
| gp 100 (71-78, 280-288) | Melanoma | I | A*0201, A11, | 5-7 |
| | | | A3, Cw8 | |
| Tyrosinase (368-376) | Melanoma | I | A*0201 | 8 |
| Tyrosinase related protein-2 | Melanoma | I | A*0201, A31, | 9-10 |
| (180-188, 197-205, 387-395) | | | A33 (A3 st) | |
| MAGE-1 (multiple peptides) | Melanoma | I | A1, A2.1, A3.2, | 11 |
| | | | A11, A24 | |
| MAGE-3 (168-176, | Melanoma | I | A*0101, | 12-13 |
| 271-279) | | | A*0201 | |
| MAGE-3 ((114-127, | Melanoma | II | DR13, DR11 | 14-15 |
| 281-295) | | | | |
| MAGE-1, 2, 3, 6 (127-136) | Melanoma | II | B*3701 | 16 |
| (promiscuous epitope) | | | | |
| MC1R melanocyte | Melanoma | II | A*0201 | 17 |
| stimulating hormone | | | | |
| receptor (244, 283, 291) | | | | |
| 707-AP | Melanoma | II | A*0201 | 18 |
| GAGE (1, 2, 3, 4, 5, 6, 7B, | Melanoma, | II | Cw6 (GAGE1) | 19 |
| 8) | others | | | |
| Her2/neu (at least 6 epitopes, | Breast, ovarian, | II | A*0201, A3 st | 21-23 |
| including 654-662, 9(754)) | pancreatic, | | | |
| | non-small cell | | | |
| | lung carc., | | | |
| | melanoma | | | |
| CEA (CAP-1), 9(61) | Colorectal | II | A3 st, A24 | 21, |
| | carc., others | | | 24 |
| Papillomavirus type 16 E7 | Cervical | II | A2*01 | 25-27 |
| (11-20, 82-90, 86-93) | squamous carc. | | | |
| Bcr-abl (4 peptides) | Chronic | II | A3, A11 | 28-29 |
| | myelogenous | | | |
| | leukemia | | | |
| p53 (149-157, 264-272) | Squamous cell | II | A2*01 | 30 |
| | carc. of the | | | |
| | head and neck | | | |
| RBP-1 (247-256, 250-259) | Breast carc. | II | A*0201, | 31 |
| | | | A*0301 | |

st: supertype

In another embodiment, the peptide is derived from an agent for infectious disease or an infected cell, or stimulates an immune response against an agent for infectious disease. Agents for infectious disease include bacteria, mycobacteria, fungi, worms, protozoa, parasites, viruses, prions, etc. Non-limiting examples of peptides derived from infectious agents are described in Table 4.

TABLE 4
Peptides derived from agents for infectious disease

| | | Rec. | | |
|---|---|---|---|---|
| Peptide antigen | Expressed in | by | HLA allele | Ref. |
| CY1899 (core | Hepatitis B | I | A2*01 | 32-33 |
| protein 18-27) | | | | |
| Nucleocapsid T | Hepatitis B | II | | 34 |
| cell epitope 18-27 | | | | |
| Non-structural | Acute hepatitis C | II | DR4, −11, −12, | 35 |
| protein 3 | | | −13, −16 | |
| (1248-1261) | | | | |
| NS4.1769 | Chronic hepatitis C | I | A2*01 | 36-37 |
| (NS4B, NS5B) | | | | |
| GroES hsp 10 | Leprosy (Mycobacterium | II | DRB5*0101 | 38 |
| (25-39, 28-42) | leprae) | | | |
| MN r gp 160 | HIV-1 | I | A2*01 | 39 |
| Tax (11-19) | HTLV-1 | I | A2*01 | 40 |
| MP (57-66) | Influenza | I | A2*01 | 41 |
| Tetanus toxin | Tetanus (Clostridium | II | DRB1*1302 | 42 |
| (830-843) | tetani) | | | |
| SSP2 | Malaria (Plasmodium | I | A2*01, multiple A | 43-44 |
| | falciparum) | | and B supertypes | |
| TSA-1, ASP-1, | Chagas' Disease | I | A2*01 | 45 |
| ASP-2 | (Trypanosoma cruzi) | | | |

Reference List for Tables 3 and 4:
1. Valmori, D. et al., J. Immunol. 161:6956-62 (1998).
2. Brinckerhoff, L. H. et al., Int. J. Cancer. 83:326-34 (1999).
3. Rivoltini, L. et al., Cancer Res. 59:301-6 (1999).
4. Zarour, H. M. et al., Proc. Natl. Acad. Sci USA. 97:400-5 (2000).
5. Castelli, C. et al., J. Immunol. 162:1739-48 (1999).
6. Abdel-Wahab, Z. et al., Cell. Immunol. 186:63-74 (1998).
7. Kawashima, I. et al., Int. J. Cancer. 78:518-24 (1998).
8. Valmori, D. et al., Cancer Res. 59:4050-5 (1999).
9. Parkhurst, M. R. et al., Cancer Res. 58:4895-901 (1998).
10. Wang, R. F. et al., J. Immunol. 160:890-7 (1998).
11. Celis, E. et al., Molecular Immunol. 31:1423-30 (1994).
12. Valmori, D. et al., Cancer Res. 57:735-41 (1997).
13. Fleischhauer, K. et al., J. Immunol. 159:2513-21 (1997).
14. Chaux, P. et al., J. Exp. Med. 189:767-78 (1999).
15. Manici, S. et al., J. Exp. Med. 189:871-6 (1999).
16. Tanzarella, S. et al., Cancer Res. 59:2668-74 (1999).
17. Salazar-Onfray, F. et al., Cancer Res. 57:4348-55 (1997).
18. Takahashi, T. et al., Clinical Cancer Res. 3:1363-70 (1997).
19. De Backer, O. et al., Cancer Res. 59:3157-65 (1999).
20. Rongcun, Y. et al., J. Immunol. 163:1037-44 (1999).
21. Kawashima, I. et al., Cancer Res. 59:431-5 (1999).
22. Kono, K. et al., Int. J. Cancer. 78:202-8 (1998).
23. Peiper, M. et al., Anticancer Res. 19:2471-5 (1999).
24. Nukaya, I. et al., Int. J. Cancer. 80:92-7 (1999).
25. Steller, M. A. et al., Clin. Cancer Res. 4:2103-9 (1998).
26. Alexander, M. et al., Am. J. Obstetrics and Gynecology 175:1586-93 (1996).
27. Ressing, M. E. et al., J. Immunol. 154:5934-43 (1995).
28. Bocchia, M. et al., Blood 87:3587-92 (1996).
29. Bocchia, M. et al., Blood 85:2680-4 (1995).
30. Chikamatsu, K. et al., Clinical Cancer Res. 5:1281-8 (1999).
31. Takahashi, T. et al., Br. J. Cancer 81:342-9 (1999).
32. Heathcote, J. et al., Hepatology 30:531-6 (1999).
33. Livingston, B. D. et al., J. Immunol 159:1383-92 (1997).
34. Bertoletti, A. et al., Hepatology 26:1027-34 (1997).
35. Diepolder, H. M. et al., J. Virol. 71:6011-9 (1997).
36. Alexander J. et al., Human Immunol. 59:776-82 (1998).
37. Battegay, M. et al., J. Virol. 69:2462-70 (1995).
38. Kim, J. et al., J. Immunol. 159:335-43 (1997).
39. Kundu, S. K. et al., AIDS Research and Human Retroviruses 14:1669-78 (1998).
40. Hollsberg, P. et al., Proc. Natl. Acad. Sci. USA. 92:4036-40 (1995).
41. Gotch, F. et al., Nature 326:881-2 (1987).
42. Boitel, B. et al., J. Immunol. 154:3245-55 (1995).
43. Doolan, D. L. et al., Immunity 7:97-112 (1997).
44. Wizel, B. et al., J. Immunol. 155:766-75 (1995).
45. Wizel, B. et al., J. Clin. Invest. 102:1062-71 (1998).

The antigenic peptide may also be derived from a target tissue from autoimmune disease or from an allergen. Compounds comprising these antigenic peptides which suppress an immune response are especially preferred.

Further, the antigenic peptide may be synthetic. The synthetic peptide may provoke an immune response against cancerous cells or virus-infected cells.

Alternatively, the synthetic peptide may downregulate an undesirable immune response, e.g., autoimmunity or allergy.

The sequence of antigenic peptide epitopes known to bind to specific MHC molecules can be modified at the known peptide anchor positions in predictable ways that act to increase MHC binding affinity. Such “epitope enhancement” has been employed to improve the immunogenicity of a number of different MHC class I or MHC class II binding peptide epitopes (Berzofsky, J. A. et al., Immunol. Rev. 170:151-72 (1999); Ahlers, J. D. et al., Proc. Natl. Acad. Sci U.S.A. 94:10856-61 (1997); Overwijk, et al., J. Exp. Med 188:277-86 (1998); Parkhurst, M. R. et al., J. Immunol. 157:2539-48 (1996)).

Antibodies are constructed of one, or several, units, each of which consists of two heavy (H) polypeptide chains and two light (L) polypeptide chains. The H and L chains are made up of a series of domains. The L chains, of which there are two major types (κ and λ), consists of two domains. The H chains molecules are of several types, including μ, δ, and γ (of which there are several subclasses), α and ε. In humans, there are eight genetically and structurally identified antibody classes and subclasses as defined by heavy chain isotypes: IgM, IgD, IgG3, IgG1, IgG2, IgG4, IgE, and IgA. Further, for example, “IgG” means an antibody of the G class, and that, “IgG1” refers to an IgG molecules of subclass 1 of the G class.

As used herein, the term “antibody” (Ab) or “monoclonal antibody” (Mab) is meant to include intact molecules as well as antibody portions (such as, for example, Fab and F(ab′)₂ portions and Fv fragments) which are capable of specifically binding to a cell surface marker. Such portions are typically produced by proteolytic cleavage, using enzymes such as papain (to produce Fab portions) or pepsin (to produce F(ab′)₂ portions). Especially preferred in the compounds of the invention are Fab portions. Alternatively, antigen-binding portions can be produced through the application of recombinant DNA technology.

The immunoglobulin can be a “chimeric antibody” as that term is recognized in the art. Also, the immunoglobulin may be a “bifunctional” or “hybrid” antibody, that is, an antibody which may have one arm having a specificity for one antigenic site, such as a tumor associated antigen while the other arm recognizes a different target, for example, a hapten which is, or to which is bound, an agent lethal to the antigen-bearing tumor cell. Alternatively, the bifunctional antibody may be one in which each arm has specificity for a different epitope of a tumor associated antigen of the cell to be therapeutically or biologically modified. In any case, the hybrid antibodies have a dual specificity, preferably with one or more binding sites specific for the hapten of choice or one or more binding sites specific for a target antigen, for example, an antigen associated with a tumor, an infectious organism, or other disease state.

Biological bifunctional antibodies are described, for example, in European Patent Publication, EPA 0 105 360, to which those skilled in the art are referred. Such hybrid or bifunctional antibodies may be derived, as noted, either biologically, by cell fusion techniques, or chemically, especially with cross-linking agents or disulfide bridge-forming reagents, and may be comprised of whose antibodies and/or fragments thereof. Methods for obtaining such hybrid antibodies are disclosed, for example, in PCT application WO83/03679, published Oct. 27, 1983, and published European Application EPA 0 217 577, published Apr. 8, 1987. Particularly preferred bifunctional antibodies are those biologically prepared from a “polydome” or “quadroma” or which are synthetically prepared with cross-linking agents such as bis-(maleimideo)-methyl ether (“BMME”), or with other cross-linking agents familiar to those skilled in the art.

In addition the immunoglobin may be a single chain antibody (“SCA”). These may consist of single chain Fv fragments (“scFv”) in which the variable light (“V[L]”) and variable heavy (“V[H]”) domains are linked by a peptide bridge or by disulfide bonds. Also, the immunoglobulin may consist of single V[H]domains (dAbs) which possess antigen-binding activity. See, e.g., G. Winter and C. Milstein, Nature 349:295 (1991); R. Glockshuber et al., Biochemistry 29:1362 (1990); and, E. S. Ward et al., Nature 341:544 (1989).

Especially preferred for use in the present invention are chimeric monoclonal antibodies, preferably those chimeric antibodies having specificity toward a tumor associated antigen. As used in this example, the term “chimeric antibody” refers to a monoclonal antibody comprising a variable region, i.e. binding region, from one source or species and at least a portion of a constant region derived from a different source or species, usually prepared by recombinant DNA techniques. Chimeric antibodies comprising a murine variable region and a human constant region are preferred in certain applications of the invention, particularly human therapy, because such antibodies are readily prepared and may be less immunogenic than purely murine monoclonal antibodies. Such murine/human chimeric antibodies are the product of expressed immunoglobulin genes comprising DNA segments encoding murine immunoglobulin variable regions and DNA segments encoding human immunoglobulin constant regions. Other forms of chimeric antibodies encompassed by the invention are those in which the class or subclass has been modified or changed from that of the original antibody. Such “chimeric” antibodies are also referred to as “class-switched antibodies”. Methods for producing chimeric antibodies involve conventional recombinant DNA and gene transfection techniques now well known in the art. See, e.g., Morrison, S. L. et al., Proc. Nat'l Acad. Sci. 81:6851 (1984).

Encompassed by the term “chimeric antibody” is the concept of “humanized antibody”, that is those antibodies in which the framework or “complementarity” determining regions (“CDR”) have been modified to comprise the CDR of an immunoglobulin of different specificity as compared to that of the parent immunoglobulin. In a preferred embodiment, a murine CDR is grafted into the framework region of a human antibody to prepare the “humanized antibody”. See, e.g., L. Riechmann et al., Nature 332:323 (1988); M. S. Neuberger et al., Nature 314:268 (1985). Particularly preferred CDR'S correspond to those representing sequences recognizing the antigens noted above for the chimeric and bifunctional antibodies. The reader is referred to the teaching of EPA 0 239 400 (published Sep. 30, 1987), for its teaching of CDR modified antibodies.

One skilled in the art will recognize that a bifunctional-chimeric antibody can be prepared which would have the benefits of lower immunogenicity of the chimeric or humanized antibody, as well as the flexibility, especially for therapeutic treatment, of the bifunctional antibodies described above. Such bifunctional-chimeric antibodies can be synthesized, for instance, by chemical synthesis using cross-linking agents and/or recombinant methods of the type described above. In any event, the present invention should not be construed as limited in scope by any particular method of production of an antibody whether bifunctional, chimeric, bifunctional-chimeric, humanized, or an antigen-recognizing fragment or derivative thereof.

In addition, the invention encompasses within its scope immunoglobulins (as defined above) or immunoglobulin fragments to which are fused active proteins, for example, an enzyme of the type disclosed in Neuberger et al., PCT application, WO86/01533, published Mar. 13, 1986. The disclosure of such products is incorporated herein by reference.

As noted, “bifunctional”, “fused”, “chimeric” (including humanized), and “bifunctional-chimeric” (including humanized) antibody constructions also include, within their individual contexts constructions comprising antigen recognizing fragments. As one skilled in the art will recognize, such fragments could be prepared by traditional enzymatic cleavage of intact bifunctional, chimeric, humanized, or chimeric-bifunctional antibodies. If, however, intact antibodies are not susceptible to such cleavage, because of the nature of the construction involved, the noted constructions can be prepared with immunoglobulin fragments used as the starting materials; or, if recombinant techniques are used, the DNA sequences, themselves, can be tailored to encode the desired “fragment” which, when expressed, can be combined in vivo or in vitro, by chemical or biological means, to prepare the final desired intact immunoglobulin “fragment”. It is in this context, therefore, that the term “fragment” is used.

Furthermore, as noted above, the immunoglobulin (antibody), or fragment thereof, used in the present invention may be polyclonal or monoclonal in nature. Monoclonal antibodies are the preferred immunoglobulins, however. The preparation of such polyclonal or monoclonal antibodies now is well known to those skilled in the art who, of course, are fully capable of producing useful immunoglobulins which can be used in the invention. See, e.g., G. Kohler and C. Milstein, Nature 256:495 (1975). In addition, hybridomas and/or monoclonal antibodies which are produced by such hybridomas and which are useful in the practice of the present invention are publicly available from sources such as the American Type Culture Collection (“ATCC”) 10801 University Boulevard, Manassas, Va. 20110-2209 or, commercially, for example, from Boehringer-Mannheim Biochemicals, P.O. Box 50816, Indianapolis, Ind. 46250.

The antibodies of the present invention may be prepared by any of a variety of methods. For example, cells expressing the cell surface marker or an antigenic portion thereof can be administered to an animal in order to induce the production of sera containing polyclonal antibodies. In a preferred method, a preparation of protein is prepared and purified as to render it substantially free of natural contaminants. Such a preparation is then introduced into an animal in order to produce polyclonal antisera of greater specific activity.

In the most preferred method, the antibodies of the present invention are monoclonal antibodies (or portions thereof). Such monoclonal antibodies can be prepared using hybridoma technology (Kohler et al., Nature 256:495 (1975); Kohler et al., Eur. J. Immunol. 6:511 (1976); Kohler et al, Eur. J. Immunol. 6:292 (1976); Hammerling et al., In: Monoclonal Antibodies and T-Cell Hybridomas, Elsevier, N.Y., pp. 563-681 (1981)). In general, such procedures involve immunizing an animal (preferably a mouse) with a protein antigen or, more preferably, with a protein-expressing cell. Suitable cells can be recognized by their capacity to bind antibody. Such cells may be cultured in any suitable tissue culture medium; however, it is preferable to culture cells in Excell hybridoma medium (JRH Biosciences, Lenexa, Kans.) with 5% fetal bovine serum. The splenocytes of such immunized mice are extracted and fused with a suitable myeloma cell line. Any suitable myeloma cell line may be employed in accordance with the present invention; however, it is preferable to employ the parent myeloma cell line (SP₂O), available from the American Type Culture Collection, 10801 University Boulevard, Manassas, Va. 20110-2209. After fusion, the resulting hybridoma cells are selectively maintained in HAT medium, and then cloned by limiting dilution as described by Wands et al., Gastroenterology 80:225-232 (1981). The hybridoma cells obtained through such a selection are then assayed to identify clones which secrete antibodies capable of binding the antigen.

It may be preferable to use “humanized” chimeric monoclonal antibodies. Such antibodies can be produced using genetic constructs derived from hybridoma cells producing the monoclonal antibodies described above. Methods for producing chimeric antibodies are known in the art. See, for review, Morrison, Science 229:1202 (1985); Oi et al., BioTechniques 4.214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).

The antibodies of the present invention may be labeled, for example, for detection or diagnostic purposes. Suitable labels for the protein-specific antibodies of the present invention are provided below. Examples of suitable enzyme labels include malate dehydrogenase, staphylococcal nuclease, delta-5-steroid isomerase, yeast-alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, and acetylcholine esterase.

Examples of suitable radioisotopic labels include ³H, ¹¹¹In, ¹²⁵I, ¹³¹I, ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ⁴⁷Sc, ¹⁰⁹Pd, etc. ¹¹¹In is a preferred isotope where in vivo imaging is used since its avoids the problem of dehalogenation of the ¹²⁵I or ¹³¹I-labeled monoclonal antibody by the liver. In addition, this radio nucleotide has a more favorable gamma emission energy for imaging (Perkins et al., Eur. J. Nucl Med. 10:296-301 (1985); Carasquillo et al., J. Nucl. Med. 28:281-287 (1987)). For example, ¹¹¹In coupled to monoclonal antibodies with 1-(P-isothiocyanatobenzyl)-DPTA has shown little uptake in non-tumorous tissues, particularly the liver, and therefore enhances specificity of tumor localization (Esteban et al., J. Nucl. Med. 28:861-870 (1987)).

Examples of suitable non-radioactive isotopic labels include ¹⁵⁷Gd, ⁵⁵Mn, ¹⁶²Dy, ⁵²Tr, and ⁵⁶Fe.

Examples of suitable fluorescent labels include an ¹⁵²Eu label, a fluorescein label, an isothiocyanate label, a rhodamine label, a phycoerythrin label, aphycocyanin label, an allophycocyanin label, an o-phthaldehyde label, and a fluorescamine label.

Examples of suitable toxin labels include diphtheria toxin, ricin, and cholera toxin.

Examples of chemiluminescent labels include a luminal label, an isoluminal label, an aromatic acridinium ester label, an imidazole label, an acridinium salt label, an oxalate ester label, a luciferin label, a luciferase label, and an aequorin label.

Examples of nuclear magnetic resonance contrasting agents include heavy metal nuclei such as Gd, Mn, and Fe.

Typical techniques for binding the above-described labels to antibodies are provided by Kennedy et al., Clin. Chim. Acta 70:1-31 (1976), and Schurs et al., Clin. Chim. Acta 81:1-40 (1977). Coupling techniques mentioned in the latter are the glutaraldehyde method, the periodate method, the dimaleimide method, the m-maleimidobenzyl-N-hydroxy-succinimide ester method, all of which methods are incorporated by reference herein.

In one embodiment, the antibody is specific for a cell surface marker of a professional antigen presenting cell. Preferably, the antibody is specific for a cell surface marker of a dendritic cell, for example, CD83, CMRF-44 or CMRF-56. The antibody may be specific for a cell surface marker of another professional antigen presenting cell, such as a B cell or a macrophage. CD40 is expressed on both dendritic cells, B cells, and other antigen presenting cells so that a larger number of antigen presenting cells would be recruited.

In another embodiment, the antibody is specific for a cell surface marker of a T cell, for example, CD28, CTLA-4 (CD 152), or CD25. The combination of TCR mediated signal from the peptide-MHC complexes (signal 1) and co-stimulator signal through CD28 (signal 2) results in strong T cell stimulation. In contrast, the combination of TCR mediated signal from the peptide-MHC complexes (signal 1) and co-stimulator signal through CTLA-4 results in the inhibition of previously activated T cells or stimulation of antigen-specific inhibitors of activation of other T cells and may be especially useful for amelioration of autoimmune responses. CD25 is an IL-2 receptor upregulated upon T cell activation. Anti-CD25 fusion proteins could, therefore, specifically target T cells in an activated state.

CTLA-4 is a molecule expressed by activated T lymphocytes with very high affinity for costimulatory molecules B7-1 and B7-2 and has been reported to mediate signals that dampen or downregulate immune responsiveness (Bluestone, J. A. J. Immunol. 158:1989 (1997)). Although in mostmurine studies CTLA-4 specific antibodies have been reported to act antagonistically to block inhibitory effects, some human CTLA-4 specific monoclonal antibodies have been described that inhibit responses of resting human CD4+ T cells (Blair, P. J. et al., J. Immunol. 160:12-15 (1998)). The mechanisms of inhibition have not been fully characterized and may be mediated by either or both a direct inhibitory effect on T cells that have upregulated expression of CTLA-4 or through activation of a subset of inhibitory T cells that express high levels of CTLA-4. In either case, simultaneous binding of CTLA-4 and T cell receptor on a T cell by a CTLA-4 specific antibody linked to a polymeric complex of the cognate peptide:MHC ligand may result in the inhibition of undesirable T cell reactivity for that peptide:MHC complex. In one embodiment, a monovalent rather than polyvalent anti-CTLA-4 specificity may be linked to monomeric or polymeric peptide:MHC complex.

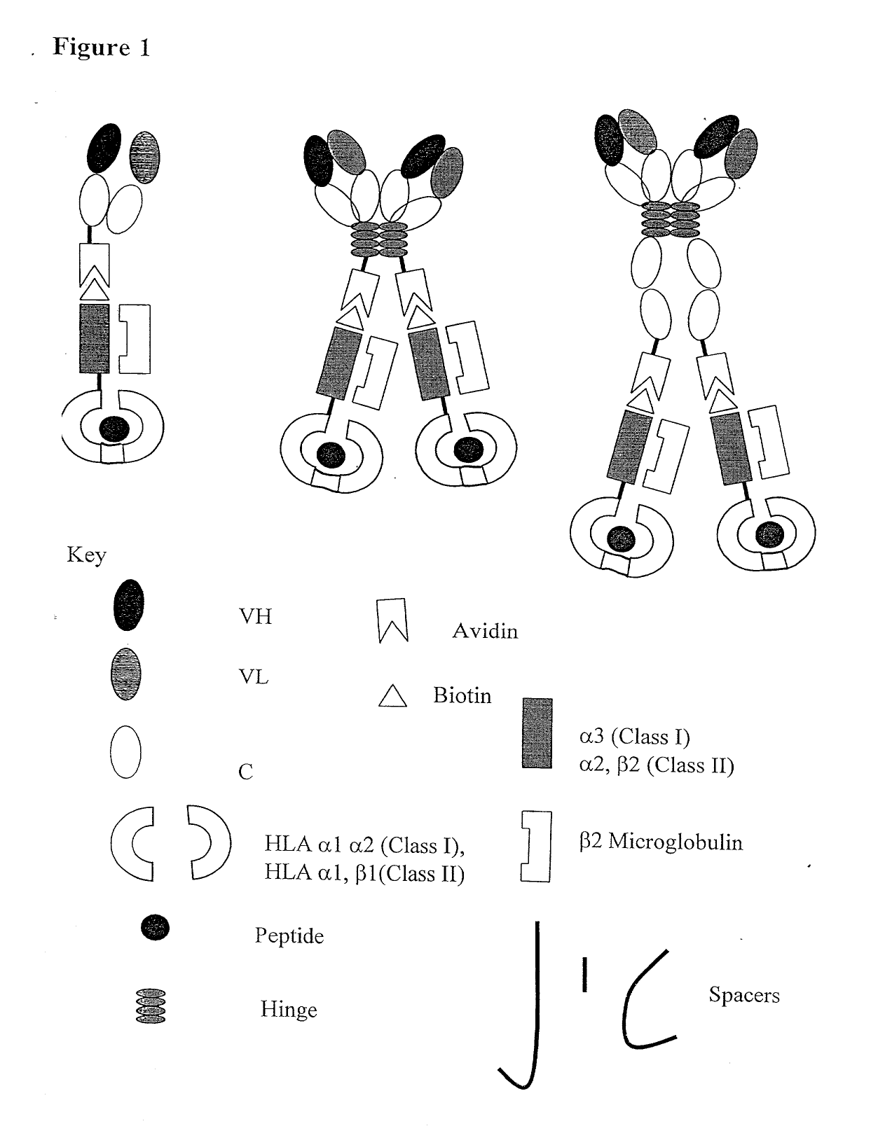

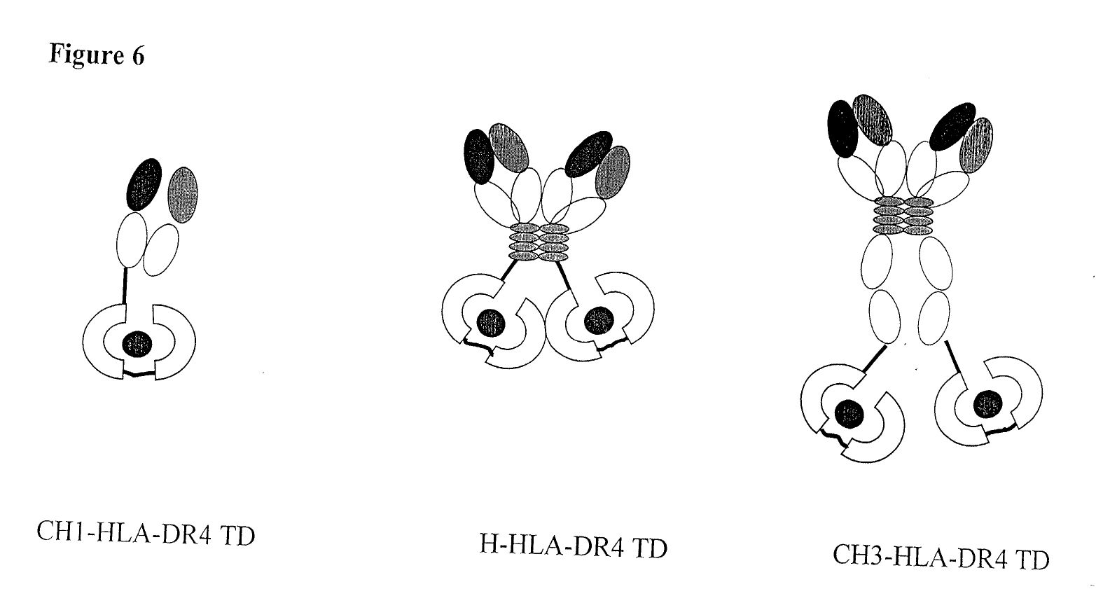

T and B lymphocytes express a variety of surface molecules that, when crosslinked by antibodies, induce positive or negative signals that culminate in responsiveness or unresponsiveness. For the purpose of antigen delivery to T and B cells, it may, in some cases, be inadvisable to crosslink a cell surface antigen with divalent or polyvalent antibody since this may induce massive cell proliferation and splenomegaly in vivo (e g. crosslinking CD3 or CD28 on T cells, or CD40 on B cells with specific antibody) or widespread cell death (anti-Fas antibody kills mice within hours of injection). Rather, it would be desirable simply to dock polymeric peptide:MHC complexes on the lymphocyte surface using compounds of the invention with only monovalent antibody specificity (see CH1 construct in FIGS. 1-6). Additional strategies for linking multimeric peptide:MHC complexes to either a monovalent or polyvalent antibody specificity are described below. The avidity of a specific T cell receptor for peptide:MHC ligands of such complexes linked to an antibody with monovalent specificity for a T cell marker would be enhanced by polymeric binding of peptide:MHC complexes as well as by linkage to the monovalent antibody specific for a second T cell membrane molecule. These targeted peptide:MHC complexes can be employed to induce proliferation or cytotoxic activity of peptide:MHC-specific T lymphocytes either in vitro or in vivo.

In another embodiment, the antibody is specific for a cell surface marker of a non-immune cell, for example, a tumor cell. Tumors evade the immune system in multiple ways, including downregulation of MHC class I and class II proteins on the surface. The compounds of the invention that specifically target tumor cells by virtue of antibody specific for antigens present on the tumor cell surface will increase presentation of peptide:MHC ligands available for specific T cell recognition and activation. One tumor surface marker, C35, is described below.

Epithelial cells and fibroblasts are non-professional antigen presenting cells. Although they express MHC class I molecules and can be induced to express MHC class II after exposure to IFN-gamma, they are not fully competent to stimulate naive T cells because they fail to express costimulatory molecules such as B7-1 and B7-2. Indeed, a signal through the T cell antigen receptor alone in the absence of a second costimulatory signal induces tolerance in naive T cells. By targeting compounds of the invention to these non-professional antigen presenting cells, it should be possible to effectively induce tolerance to the immunodominant peptide:MHC complexes of interest. A commercially available antibody, Ber-EP4 (Latza, U. et al., J. Clin. Pathol. 43:213-9 (1990), DAKO), reacts with two glycoproteins expressed on the surface of all epithelial cells except superficial squamous epithelial cells, hepatocytes, and parietal cells and has similar reactivity to HEA 125 (Moldenhauer, G. et al., Br. J. Cancer. 56:714-21 (1987)). Fibroblast-specific surface markers and antibodies that target them are under investigation in numerous laboratories and one potential candidate has been identified (Feams, C and Dowdle, E B. Int. J. Cancer. 50:621-7 (1992), Miltenyi Biotech) that could be similarly employed to promote T cell unresponsiveness to linked monomeric or polymeric peptide:MHC complexes. It is possible that for this specific application monomeric peptide:MHC complexes that do not crosslink T cell receptors on the membrane of specific cells could prove more effective than polymeric peptide:MHC complexes.

It has been reported that the liver is a site of accumulation of activated T lymphocytes about to undergo activation induced cell death (AICD) and that sinusoidal endothelial cells and Kupffer cells may constitute a “killing field” for activated CD8⁺ T cells originating from peripheral lymphoid organs (Mehal, Juedes and Crispe, J. Immunol. 163:3202-3210 (1999); Crispe, I. N. Immunol. Res. 19:143-57 (1999)). Compounds of the invention can promote trapping and deletion of specific T cells in the liver by targeting specific peptide:MHC complexes to the liver with anti-hepatocyte specific antibodies.

In a preferred embodiment, the immune system's extraordinary power to eradicate pathogens is redirected to target an otherwise evasive tumor. The immune response to commonly encountered pathogens (eg influenza virus) and/or pathogens against which individuals are likely to have been vaccinated (eg influenza, or tetanus) is associated with induction of a high frequency of high avidity T cells that are specific for immunodominant peptide:MHC complexes of cells infected with these pathogens. These same highly represented, high avidity T cells can be redirected to tumors by linking the dominant peptide:MHC ligands recognized by these T cells to a tumor-specific antibody specificity. Redirection of specific T cell activity to tumor cells through antibody targeted peptide:MHC complexes may proceed through two mechanisms. T cells either directly recognize antibody linked peptide:MHC complexes displayed on the tumor surface, or such targeted complexes are internalized and the associated peptides are represented by MHC molecules endogenous to the tumor cell. Direct T cell recognition of the targeted complex can be demonstrated by employing T cells restricted to an MHC molecule that is not endogenous to the target cell.

Non-limiting examples of cell surface markers appropriate for immune targeting of the compounds of the present invention are presented in Tables 5 and 6.

TABLE 5
Human leukocyte differentiation antigens

| Surface Antigen | Expressed by | Ref. |
|---|---|---|
| CD2 | T lymphocytes | 1-2 |
| CD4 | T cell subset | 1 |
| CD5 | T lymphocytes | 1 |
| CD6 | T lymphocytes | 1, 3 |
| CD8 | T cell subset | 1 |
| CD27 | Naïve CD4 T cell subset | 4 |
| CD31 | Naïve CD4 T cell subset | 4 |
| CD25 | Activated T cells | 1 |
| CD69 | Activated T cells | 1, 5, 6 |
| HLA-DR | Activated T cells, APC | 7 |
| CD28 | T lymphocytes | 8 |
| CD152 (CTLA-4) | Activated T cells | 9 |
| CD154 (CD40L) | Activated T cells | 10 |
| CD19 | B lymphocytes | 1, 11 |
| CD20 | B lymphocytes | 1 |
| CD21 | B lymphocytes | 1 |
| CD40 | Antigen presenting cells | 12-13 |
| CD134 (OX40) | Antigen presenting cells | 13-14 |
| B7-1 and 2 | Antigen presenting cells | 13, 15, 16 |
| CD45 | Leukocytes | 1 |
| CD83 | Mature dendritic cells | 17 |
| CMRF-44 | Mature dendritic cells | 18 |
| CMRF-56 | Mature dendritic cells | 19 |
| OX40L | Dendritic cells | 20 |
| DEC-205 | Dendritic cells | 21 |
| TRANCE/RANK receptor | Dendritic cells | 22 |

Reference listing for table 5:
1. Knapp, W. et al., eds., Leukocyte Typing IV: White Cell Differentiation Antigens, Oxford University Press, New York. (1989).
2. Bierer, B. E. et al., Seminars in Immunology. 5:249-61 (1993).
3. Rasmussen, R. A. et al., J. Immunol. 152:527 (1994).
4. Morimoto, C. et al., Clin. Exp. Immunol. 11:241-7 (1993).
5. Ziegler, S. F. et al., Stem Cells 12:456-65 (1994).
6. Marzio, R. et al., CD69 and regulation of immune function. 21:565-82 (1999).
7. Rea, I. M. et al., Exp. Gerontol. 34:79-93 (1999).
8. June, C. H. et al., Immunology Today 11:211 (1993).
9. Lindsten, T. et al., J. Immunol. 151:3489 (1993).
10. Mackey, M. F. et al., J. Leukocyte Biol. 63:418-28 (1998).
11. Bradbury, L. E. et al., J. Immunol. 151:2915 (1993).
12. Clark, E. A., and Ledbetter, J. A., Proc. Natl. Adad. Sci. USA. 83:4494 (1986).
13. Schlossman, S. et al., eds. Leukocyte Typing V: White Cell Differentiation Antigens. Oxford University Press, New York (1995).
14. Latza, U. et al., Eur. J. Immunol. 24:677 (1994).
15. Koulova, L. et al., J. Exp. Med. 173:759 (1991).
16. Azuma, M. et al., Nature 366:76 (1993).
17. Zhou, L. J., and Tedder, T. F., J. Immunol. 154:3821 (1995).
18. Vuckovic, S. et al., Exp. Hematology 26:1255 (1998).
19. Hock, B. D. et al., Tissue Antigens 53:320-34 (1999).
20. Chen, A. I. et al., Immunity 11:689 (1999).
21. Kato, M. et al., Immunogenetics. 47:442 (1998).
22. Anderson, D. M. et al., Nature 390:175 (1997).

TABLE 6
Tumor cell surface antigens recognized by antibodies

| Antigen(s) | Expressed in | Ref. |
|---|---|---|
| CEA | Colorectal, thyroid carcinoma, others | 1-6 |
| Her2/neu | Breast, ovarian carcinomas | 7 |
| CM-1 | Breast | 8 |
| MUC-1 | Pancreatic carcinoma, others | 9-10 |
| 28K29 | Lung adenocarcinoma, large cell | 11 |
| | carcinoma | |
| E48 | Head and neck squamous cell | 12 |
| | carcinoma | |
| U36 | Head and neck squamous cell | 12 |
| | carcinoma | |
| NY-ESO-1* | Esophageal carcinoma, melanoma, | 13-14 |
| | others | |
| KU-BL 1-5* | Bladder carcinoma | 15 |
| NY CO 1-48* | Colon carcinoma | 16 |
| HOM MEL 40* | Melanoma | 17 |
| OV569 | Ovarian carcinoma | 18 |
| ChCE7 | Neuroblastoma, renal cell carcinoma | 19 |
| CA19-9 | Colon carcinoma | 20 |
| CA125 | Ovarian carcinoma | 21 |
| Gangliosides (GM2, | Melanoma, neuroblastoma, others | 22 |
| GD2, 9-o-acetyl-GD3, | | |
| GD3) | | |

*Antigens identified using SEREX technology.
Reference List for Table 6:
1. Juweid, M. E. et al., Cancer 85:1828-42 (1999).
2. Stewart, L. M. et al., Imunotherapy 47:299-306 (1999).
3. Robert, B. et al., International J. Cancer 81:285-91 (1999).
4. Kraeber-Bodere, F. et al., J. Nuclear Medicine 40:198-204 (1999).
5. Kawashima, I. et al., Cancer Res. 59:431-5 (1999).
6. Nasu, T. et al., Immunology Letters 67:57-62 (1999).
7. Zhang, H. et al., Experimental & Molecular Pathology 67:15-25 (1999).
8. Chen, L. et al., Acta Academiae Medicinae Sinicae 19(2):150-3.
9. Beum, P. V. et al., J. Biol. Chem. 274:24621-8 (1999).
10. Koumarianou A. A. et al., British J. Cancer 81:431-9 (1999).
11. Yoshinari, K. et al., Lung Cancer 25:95-103 (1999).
12. Van Dongen, G. A. M. S. et al., Anticancer Res. 16:2409-14 (1996).
13. Jager, E. et al., J. Exp. Med. 187:265-70 (1998).
14. Jager, E. et al., International J. Cancer 84:506-10 (1999).
15. Ito, K. et al., AUA 2000 Annual Meeting, Abstract 3291 (2000).
16. Scanlan, M. J. et al., International J. Cancer 76:652-8 (1998).
17. Tureci, O. et al., Cancer Res. 56:4766-72 (1996).
18. Scholler, N. et al., Proc. Natl. Acad, Sci. USA 96:11531-6 (1999).
19. Meli, M. L. et al., International J. Cancer 83:401-8 (1999).
20. Han, J. S. et al., Cancer 76:195-200 (1995).
21. O'Brien, T. J. et al., International J. Biological Markers 13:188-95 (1998).
22. Zhang, S. et al., Cancer Immunol. Immunotherapy 40:88-94 (1995).

The conjugation of the MHC-peptide complex(es) to the antibody may be conducted in any suitable manner. For example, the coupling may be of a physical and/or chemical type. The antibody and MHC-peptide complex may be coupled physically utilizing a carrier for example a Sepharose carrier (available from Pharmacia, Uppsala, Sweden) or recently developed microsphere technology. (Southern Research Institute).

Alternatively, the MHC molecules may be linked together directly. A number of reagents capable of cross-linking proteins are known in the art, illustrative entities include: azidobenzoyl hydrazide, N-[4-(p-azidosalicylamino)butyl]-3′-[2′-pyridyldithio]propionamide), bis-sulfosuccinimidyl suberate, dimethyladipimidate, disuccinimidyltartrate, N-γ-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, formaldehyde and succinimidyl 4-[N-maleimidomethyl]cyclohexane-1-carboxylate.

Alternatively, the MHC complex can be genetically modified by including sequences encoding amino acid residues with chemically reactive side chains such as Cys or His. Such amino acids with chemically reactive side chains may be positioned in a variety of positions of a MHC complex, preferably distal to the antigenic peptide and binding domain of the MHC complex. For example, the C-terminus of the β chain of an MHC class II molecule distal from the antigenic peptide suitably may contain such reactive amino acid(s). Suitable side chains can be used to chemically link two or more MHC-peptide complexes to a suitable dendrimer particle. Dendrimers are synthetic chemical polymers that can have any one of a number of different functional groups on their surface (D. Tomalia, Aldrichimica Acta 26:91:101 (1993)). Exemplary dendrimers for use in accordance with the present invention include e.g. E9 starburst polyamine dendrimer and E9 combburst polyamine dendrimer, which can link cysteine residues.

A short linker amino acid sequence may be inserted between the MHC-peptide complex(es) and the antibody. The length of the linker sequence will vary depending upon the desired flexibility to regulate the degree of antigen binding and cross-linking. If a linker sequence is included, this sequence will preferably contain at least 3 and not more than 30 amino acids. More preferably, the linker is about 5, 10, 15, 20, or 25 amino acids long. Generally, the linker consists of short glycine/serine spacers, but any known amino acid may be used.

The biotin binding sites in chicken avidin are arranged in a tetrahedral array such that three of any four bound peptide:MHC complexes are displayed on one face of the tetrahedron to contact the T cell membrane (McMichael, A. J. and O'Callaghan, C. A. J. Exp. Med. 187:1367-71 (1998)). This display configuration may be advantageous to promote the T cell clustering required for activation (Boniface, J. J. et al., Immunity 9:459-66 (1998)). There are, however, alternative means of linking polymeric peptide:MHC complexes to an antibody specificity that might be substituted for the tetrahedral array. Described below are direct fusion of heterodimeric or single chain MHC class I and class II molecules to the carboxyl end of an antibody immunoglobulin chain or fragment thereof. Fusion of MHC molecules to the amino terminus of the immunoglobulin chain variable regions has been previously described (Dal Porto, J. et al., Proc. Natl. Acad. Sci., USA 90:6671-75 (1993)). Although this fusion product does not interfere with recognition of haptens in fusion products with hapten-specific antibody, the proximity of peptide:MHC complex and antibody binding site makes it more likely that the peptide:MHC complex could interfere with antibody binding to macromolecular determinants embedded in a complex membrane. Moreover, while fusion of MHC molecules to the amino terminus of immunoglobulin or immunoglobulin fragments preserves the Fc binding function for optimal presentation of peptide:MHC complex by Fc receptor expressing cells, the relative orientation of antibody binding site and peptide:MHC complex is far less favorable for antigen presentation to T cells by cells that might be targeted by the specific antibody (Hamad, A. R. A. et al., J. Exp. Med. 188:1633-40 (1998); Greten, T. F. et al., Proc. Natl. Acad. Sci., USA 95:7568-73 (1998); Casares, S. et al, J. Exp. Med. 190:543-553 (1999)). There is, therefore, a need for new compounds that can serve the requirements of targeted delivery of polymeric peptide:MHC ligand to T cells and their antigen-specific receptor. Localization of the MHC molecule at the carboxyl terminus of immunoglobulin chains serves this purpose. The peptide:MHC complex is well separated from the antibody binding site and is unlikely to interfere with its targeting specificity.

MHC molecules fused to the carboxyl terminus of the exceptionally long IgG3 hinge region or to the CH3 domain, are especially far removed from possible interference with the antigen binding site or its ligand. Moreover, the preferred embodiments of the compounds of this invention promote antibody mediated targeting to antigen presenting cells or tumors in a way which properly orients polymeric peptide:MHC complexes for presentation to T cells and their antigen-specific receptors. As depicted in FIGS. 1-6, Fc binding function is preserved in the compounds of this invention that are based on CH3 fusions. It is possible that this would extend the half-life of these compounds in vivo.

Direct fusion of MHC molecules to the termini of IgG heavy chains or fragments thereof are limited to dimeric peptide:MHC complexes. Boniface, J. J. et al., Immunity 9:459-66 (1998) have suggested that trimeric peptide:MHC complexes provide a much more potent stimulus for T cell activation. Previous reports of specific T cell activation with dimeric peptide:MHC complexes fused to the amino terminus of immunoglobulin chains might be attributed to immobilization on plastic (Hamad, A. R. A. et al., J. Exp. Med. 188:1633-40 (1998)), or to the presence of FcR positive antigen presenting cells and restriction to a limited Th2 type response in vivo (Casares, S. et al., J. Exp. Med. 190:543-553 (1999)). Cochran, J. R. et al., Immunity 12:241-50(2000) suggest that dimers of another type of peptide:MHC complex are as effective as trimers or tetramers for triggering early T cell activation events. It is possible that these disparate results regarding the relative efficacy of dimers and higher order oligomers for triggering early T cell activation events is related to the binding avidity of specific complexes for T cell receptors. In any case, following the initial peptide:MHC and T cell receptor trimolecular triggering events there remains a need for costimulation to drive optimal T cell expansion and expression of the full range of effector functions. The compounds of this invention allow the advantages of polymeric peptide:MHC triggering complexes to be combined with targeting to costimulation competent antigen presenting cells or, in the case of anti-CD28, direct antibody mediated costimulation.

There are several other ways to assemble polymeric MHC molecules on a targeting antibody besides direct antibody-MHC fusion or the binding of biotinylated MHC molecules to antibody-avidin fusion proteins. Cochran, J. R. et al., Immunity 12:241-50 (2000) describe the use of chemically synthesized peptide-based cross-linking reagents in which two or more thiol-reactive maleimide groups are linked to lysine side chains in a flexible peptide of 8 to 19 residues containing glycine, serine, and glutamic acid in addition to the modified lysine residues. One chain of an HLA class II molecule is modified to introduce a cysteine residue at the carboxyl terminus. Following synthesis in E. coli, a complete cysteine modified HLA class II molecules is assembled in vitro in the presence of peptide. Cysteine modified HLA molecules react with the maleimide groups on the various peptide backbones with either two, three, or four modified lysine residues for formation of peptide:MHC dimers, trimers, and tetramers. Similar oligomers could be assembled with HLA class I molecules. In a preferred embodiment, a carboxyl terminal cysteine modified immunoglobulin chain or fragment thereof could also be synthesized for reaction with a maleimide-modified lysine residue on the same backbone peptide and at the same time as the cysteine modified HLA molecules. This strategy could, for example, be employed to link polymeric peptide:MHC complexes to the monovalent CH1 antibody fragment depicted in FIGS. 1-6.

Alternatively or in addition, the MHC-peptide complex(es) and antibody may be linked through a multivalent compound, for example, chicken avidin or streptavidin (Shin, S. U. et al., J. Immunology 158: 4797-4804 (1997)) to which biotinylated peptide:MHC complexes are bound (Altman, J. et al, Science 274:94-96 (1996); Boniface, J. J. et al., Immunity 9:459-66 (1998)); or a leucine zipper system. Cochran, J. R. et al., Immunity 12:241-50 (2000) describe the use of chemically synthesized peptide-based cross-linking reagents in which two or more thiol-reactive maleimide groups are linked to lysine side chains in a flexible peptide of 8 to 19 residues containing glycine, serine, and glutamic acid in addition to the modified lysine residues. An HLA molecule is modified to introduce a cysteine residue at the carboxyl terminus. Cysteine modified HLA molecules react with the maleimide groups on the various peptide backbones with either two, three, or four modified lysine residues for formation of peptide:MHC dimers, trimers, and tetramers. Pack, P., et al. J. Mol. Biol. 246:28-34 (1995) constructed tetravalent miniantibodies by fusing a modified GCN4-zipper that results in formation of highly stable trimeric and tetrameric structures to the carboxyl terminus of a single-chain Fv fragment via a flexible hinge region.

Yet another means of assembling polymeric peptide:MHC complexes on specific antibody is to exploit the observation that defined amino acid substitutions in the GCN4 leucine zipper dimerization domain results in formation of highly stable trimeric and tetrameric structures of the synthetic peptide (Harbury, P. B. et al., Science 262:1401-7 (1993)). Pack, P., et al. J. Mol. Biol. 246:28-34 (1995) constructed tetravalent miniantibodies by fusing the modified GCN4-zipper to the carboxyl terminus of a single-chain Fv fragment via a flexible hinge region. Several additional modifications of the fusion protein improved yield from bacterial synthesis. Addition of a carboxyl terminal tag would facilitate purification. Targeted tetravalent peptide:MHC complexes could be assembled from a mixture of single chain antibody and single chain MHC molecules each separately fused through a hinge region to the modified GCN4-zipper motif.

In preferred embodiments of the invention, the compound further comprises a cytokine or lymphokine attached to the multivalent compound. Cytokines or lymphokines include, but are not limited to, interleukins (e.g., IL-2, IL-3, IL-4, IL-5, IL-6, IL-10, IL-12, IL-15, and IL-18), a interferons (e.g., IFNα), β interferons (e.g., IFNβ), γ interferons (e.g., IFNγ), granulocyte-macrophage colony stimulating factor (GM-CSF), and transforming growth factor (TGF, e.g., TGFα and TGFβ).

The alternative embodiments of this invention, direct fusion of antibody and MHC molecules or indirect association of antibody and peptide:MHC complexes through a multivalent entity, are respectively advantageous in different situations. The direct fusion simplifies production of the compound while the multivalent entity, as indicated above, can present a larger number of more diverse ligands. In both cases it is desirable to design products that induce minimal immune reactivity. In the case of direct immunoglobulin-MHC fusion proteins, this is accomplished by employing species compatible antibodies and MHC molecules joined by simple linkers with a relatively non-immunogenic composition. Multivalent entities may be similarly selected to minimize immunogenicity. Chicken avidin is thought to be relatively nonimmunogenic because of its high concentration in egg products and the well-known propensity of oral infusion to induce immune tolerance (Shin, S. U. et al., J. Immunology 158: 4797-4804 (1997)). It may, in addition, be possible to develop protocols, including some that employ compounds of this invention, that induce specific tolerance.

The attachment site on the MHC-peptide complex or antibody for binding to a multivalent compound may be naturally occurring, or may be introduced through genetic engineering. The site will be a specific binding pair member or one that is modified to provide a specific binding pair member, where the complementary pair has a multiplicity of specific binding sites. Binding to the complementary binding member can be a chemical reaction, epitope-receptor binding or hapten-receptor binding where a hapten is linked to the subunit chain.

In a preferred embodiment, one of the MHC chains contains an amino acid sequence which is a recognition site for a modifying enzyme. Preferably, the recognition site is near the carboxyl terminus of the MHC molecule. Modifying enzymes include BirA, various glycosylases, farnesyl protein transferase, and protein kinases. The group introduced by the modifying enzyme, e.g. biotin, sugar, phosphate, farnesyl, etc. provides a complementary binding pair member, or a unique site for further modification, such as chemical cross-linking, biotinylation, etc. that will provide a complementary binding pair member.

For example, the MHC molecule may be engineered to contain a site for biotinylation, for example a BirA-dependent site. Preferably, the site for biotinylation is at or near the carboxyl terminus. The antibody or fragment thereof can be linked to avidin either directly or indirectly. Direct linkage is accomplished by making an antibody-avidin fusion protein through genetic engineering as described in, for example, Shin et al., Shin, S.-U. et al., J. Immunol. 158:4797-4804 (1997); and Penichet et al., J. Immunol. 163:4421-4426. In another embodiment, indirect linkage can be effected by employing the previously described construct incorporating genes for the heavy and light chain variable regions of an antibody specific for the hapten dansyl (Shin, S.-U. et al., J. Immunol. 158:4797-4804 (1997)). MHC-peptide complexes assembled on the antidansyl-avidin fusion protein could then associate with any dansylated antibody with the desired targeting specificity. Dansyl chloride (DNS, Molecular Probes cat #D21, 5-dimethylaminonapthalene-1-sulfonyl chloride) is freshly dissolved in dimethyl formamide, 0.1-1 mg/ml. DNS solution (1 μl) is added to 10 μl (20 μg) of purified antibody (2 mg/ml) dissolved in 0.1M NaHCO₃. After one hour incubation at 4° C. with rotation, the reaction is quenched with 2 μl of 0.1M glycine. For each antibody, it is necessary to titrate the DNS concentration to empirically determine the amount necessary to label the antibody while still retaining antibody specificity.

In one embodiment, the compound of the invention incorporates an antibody specificity for a particular immunoglobulin class or isotype, in a preferred embodiment this is an IgG isotype whose expression is regulated by cytokines secreted by Th1 type T cells, compounds of the invention with this immunoglobulin isotype specificity will bind antigen-specific humoral antibodies of this isotype. The bound humoral antibody will, as a result, target the linked peptide:MHC complex and any linked cytokines to those cells that express the specific foreign antigens or autoantigens that were responsible for inducing this specific antibody response. The rationale is that, without prior knowledge of the specific antigens targeted in this cancer or infectious disease, it will be possible to deliver desired markers or signals to eradicate the cellular source of specific antigen.

Additionally, the MHC may form a fusion protein with the antibody. Fusion antibodies can be made using conventional recombinant nucleic acid techniques. The fusion may be direct or may contain spacers. The fusion proteins are comprised of an MHC-peptide complex attached to the carboxyl terminus of an antibody or fragment thereof, wherein the antibody or fragment thereof is specific for a cell surface marker. Methods of making MHC-antibody fusion proteins are described in, for example, Dal Porto et al., Proc. Natl. Acad. Sci. USA 90:6671-6675 (1993) and Hamad et al., J. Exp Med. 188:1633-1640 (1998).

In certain embodiments, the MHC-peptide complex comprises an MHC class I α chain or fragment thereof, a β₂-microglobulin molecule or fragment thereof, and an antigenic peptide. The MHC-peptide complex may be attached to the antibody at the light chain or the heavy chain of the antibody, or both. The MHC class I α chain may be attached to either the light chain or the heavy chain of the antibody, and/or the β₂-microblogulin molecule may be attached to either the light chain and/or the heavy chain of the antibody. For example, in certain embodiments, the MHC class I α chain is attached to the heavy chain of the antibody; the MHC class I α chain is attached to the heavy chain of the antibody, and the β₂-microblogulin molecule is attached to the light chain of the antibody; the MHC class I α chain is attached to the light chain of the antibody; the MHC class I α chain is attached to the light chain of the antibody and the β₂-microblogulin molecule is attached to the heavy chain of the antibody; the β₂-microblogulin molecule is attached to the light chain of the antibody; or the β₂-microblogulin molecule is attached to the heavy chain of the antibody.

In certain other embodiments, the MHC-peptide complex comprises an MHC class II α chain, or fragment thereof, an MHC class II β chain, or fragment thereof, and an antigenic peptide. The MHC class II α chain may be attached to either the light chain or the heavy chain of the antibody and/or the MHC class II β chain may be attached to either the light chain and/or the heavy chain of the antibody. For example, in certain embodiments, the MHC class II α chain is attached to the light chain of the antibody; the MHC class II β chain is attached to the light chain of the antibody; the MHC class II α chain is attached to the heavy chain of the antibody; the MHC class II chain is attached to the heavy chain of the antibody; the MHC class II α chain is attached to the light chain of the antibody and the MHC class II β chain is attached to the heavy chain of the antibody; or the MHC class II α chain is attached to the heavy chain of the antibody and the MHC class II β chain is attached to the light chain of the antibody.

The present invention also relates to vectors which include a nucleotide sequence encoding a compound of the present invention or parts thereof, host cells which are genetically engineered with the recombinant vectors, and the production of the compounds of the present invention or parts thereof by recombinant techniques.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The DNA insert should be operatively linked to an appropriate promoter, such as the phage lambda PL promoter, the E. coli lac, trp and tac promoters, the SV40 early and late promoters and promoters of retroviral LTRs, to name a few. Other suitable promoters will be known to the skilled artisan. The expression constructs will further contain sites for transcription initiation, termination and, in the transcribed region, a ribosome binding site for translation. The coding portion of the mature transcripts expressed by the constructs will preferably include a translation initiating at the beginning and a termination codon (UAA, UGA or UAG) appropriately positioned at the end of the polypeptide to be translated.

As indicated, the expression vectors will preferably include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Appropriate culture mediums and conditions for the above-described host cells are known in the art. For example, MHC class I molecules can be expressed in Drosophila cells (U.S. Pat. No. 6,001,365).

Among vectors preferred for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986).

The polypeptide may be expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred fusion protein comprises a heterologous region from immunoglobulin that is useful to solubilize proteins. For example, EP-A-0 464 533 (Canadian counterpart 2045869) discloses fusion proteins comprising various portions of constant region of immunoglobin molecules together with another human protein or part thereof. In many cases, the Fc part in a fusion protein is thoroughly advantageous for use in therapy and diagnosis and thus results, for example, in improved pharmacokinetic properties (EP-A 0232262). On the other hand, for some uses it would be desirable to be able to delete the Fc part after the fusion protein has been expressed, detected and purified in the advantageous manner described. This is the case when the Fc portion proves to be a hindrance to use in therapy and diagnosis, for example when the fusion protein is to be used as an antigen for immunizations. In drug discovery, for example, human proteins, such as the hIL5-receptor, have been fused with Fc portions for the purpose of high-throughput screening assays to identify antagonists of hIL-5. See, D. Bennett et al., J. Mol. Recognition 8:52-58 (1995) and K. Johanson et al., J. of Biol. Chem. 270(16):9459-9471 (1995).

Several reports have described secretion and assembly of fusion proteins comprised of diverse sequences linked to the carboxyl terminus of immunoglobulin chains (Harvill, E. T. et al., J. Immunol. 157:3165-70 (1996); Shin, S. U. et al., J. Immunology 158: 4797-4804 (1997); Penichet, M. L. et al., J. Immunol. 163:4421-26 (1999); Zhang, H. F. et al., J. Clin. Invest 103:55-61 (1999)). Fusion proteins of the compounds of this invention will likewise retain amino terminal sequences of the immunoglobulin chain that direct secretion. MHC molecules linked to the carboxyl terminus of the immunoglobulin chains are stripped of hydrophobic transmembrane sequences and should not interfere with secretion.

The polypeptide can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Polypeptides useful in the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

The ability of a compound of the present invention to modulate an immune resonse can be readily determined by an in vitro assay. T cells for use in the assays include transformed T cell lines, such as T cell hybridomas, or T cells which are isolated from a mammal, e.g., from a human or from a rodent such as a mouse. T cells can be isolated from a mammal by known methods. See, for example, Shimonkevitz et al., J. Exp. Med. 158:303 (1983).

A suitable assay to determine if a compound of the present invention is capable of modulating the activity of T cells is conducted by coculturing T cells and antigen presenting cells, adding the particular compound of interest to the culture medium, and measuring IL-2 production. A decrease in IL-2 production over a standard indicates the compound can suppress an immune response. An increase in IL-2 production over a standard indicates the compound can stimulate an immune response.

The T cells employed in the assays are incubated under conditions suitable for proliferation. For example, a DO 11.10 T cell hybridoma is suitably incubated at about 37° C. and 5% CO₂ in complete culture medium (RPMI 1640 supplemented with 10% FBS, penicillin/streptomycin, L-glutamine and 5×10⁻⁵ M 2-mercaptoethanol). Serial dilutions of the compound can be added to the T cell culture medium. Suitable concentrations of the compound added to the T cells typically will be in the range of from 10⁻¹² to 10⁻⁶ M. Use of antigen dose and APC numbers giving slightly submaximal T cell activation is preferred to detect inhibition of T cell responses by the compounds of the invention.

Alternatively, rather than measurement of an expressed protein such as IL-2, modulation of T cell activation can be suitably determined by changes in antigen-dependent T cell proliferation as measured by radiolabelling techniques as are recognized in the art. For example, a labeled (e.g., tritiated) nucleotide may be introduced to an assay culture medium. Incorporation of such a tagged nucleotide into DNA serves as a measure of T cell proliferation. This assay is not suitable for T cells that do not require antigen presentation for growth, e.g., T cell hybridomas. A difference in the level of T cell proliferation following contact with the compound of the invention indicates the complex modulates activity of the T cells. For example, a decrease in T cell proliferation indicates the compound can suppress an immune response. An increase in T cell proliferation indicates the compound can stimulate an immune response.

Additionally, the ⁵¹Cr release assay, described below, can be used to determine CTL activity.

These in vitro assays can be employed to select and identify peptide that are capable of modulating an immune response. Assays described above, e.g., measurement of IL-2 production or T cell proliferation, are employed to determine if contact with the compound modulates T cell activation.

In vivo assays also may be suitably employed to determine the ability of a compound of the invention to modulate the activity of T cells. For example, a compound of interest can be assayed for its ability to inhibit immunoglobulin class switching (i.e. IgM to IgG). See, e.g., Linsley et al., Science 257:792-795 (1992)). For example, a compound of the invention can be administered to a mammal such as a mouse, blood samples obtained from the mammal at the time of initial administration and several times periodically thereafter (e.g. at 2, 5 and 8 weeks after administration). Serum is collected from the blood samples and assayed for the presence of antibodies raised by the immunization. Antibody concentrations may be determined.

The present invention also includes pharmaceutical compositions comprising a compound described above in combination with a suitable pharmaceutical carrier. Such compositions comprise a therapeutically effective amount of the compound and a pharmaceutically acceptable carrier or excipient. Such a carrier includes but is not limited to saline, buffered saline, dextrose, water, glycerol, ethanol, and combinations thereof. The formulation should suit the mode of administration.

The present invention also includes a method of modulating, i.e., either stimulating or inhibiting an immune response, comprising administering to an animal and effective amount of a compound or composition of the invention.

The compounds of the present invention may be administered in pharmaceutical compositions in combination with one or more pharmaceutically acceptable excipients. It will be understood that, when administered to a human patient, the total daily usage of the pharmaceutical compositions of the present invention will be decided by the attending physician within the scope of sound medical judgment. The specific therapeutically effective dose level for any particular patient will depend upon a variety of factors including the type and degree of the response to be achieved; the specific composition of another agent, if any, employed; the age, body weight, general health, sex and diet of the patient; the time of administration, route of administration, and rate of excretion of the composition; the duration of the treatment; drugs (such as a chemotherapeutic agent) used in combination or coincidental with the specific composition; and like factors well known in the medical arts. Suitable formulations, known in the art, can be found in Remington's Pharmaceutical Sciences (latest edition), Mack Publishing Company, Easton, Pa.

The compound to be used in the therapy will be formulated and dosed in a fashion consistent with good medical practice, taking into account the clinical condition of the individual patient (especially the side effects of treatment with the compounds alone), the site of delivery of the compound, the method of administration, the scheduling of administration, and other factors known to practitioners. The “effective amount” of the compounds of the invention for purposes herein is thus determined by such considerations.

Pharmaceutical compositions of the invention may be administered orally, intravenously, rectally, parenterally, intracisternally, intradermally, intravaginally, intraperitoneally, topically (as by powders, ointments, gels, creams, drops or transdermal patch), bucally, or as an oral or nasal spray. The term “parenteral” as used herein refers to modes of administration which include intravenous, intramuscular, intraperitoneal, intrasternal, subcutaneous and intraarticular injection and infusion.

The pharmaceutical compositions are administered in an amount which is effective for treating and/or prophylaxis of the specific indication. In most cases, the dosage is from about 1 μg/kg to about 30 mg/kg body weight daily, taking into account the routes of administration, symptoms, etc. However, the dosage can be as low as 0.001 μg/kg.

As a general proposition, the total pharmaceutically effective amount of the compositions administered parenterally per dose will be in the range of about 1 μg/kg/day to 100 mg/kg/day of patient body weight, although, as noted above, this will be subject to therapeutic discretion. If given continuously, the composition is typically administered at a dose rate of about 1 μg/kg/hour to about 5 mg/kg/hour, either by 1-4 injections per day or by continuous subcutaneous infusions, for example, using a mini-pump. An intravenous bag solution or bottle solution may also be employed.

The compounds of the invention may also suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (U. Sidman et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Sustained-release compositions also include liposomally entrapped compositions of the present invention. Liposomes are prepared by methods known per se: DE 3,218,121; Epstein, et al., Proc. Natl. Acad. Sci. USA 82:3688-3692 (1985); Hwang et al., Proc. Natl. Acad. Sci. USA 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal therapy.

For parenteral administration, in one embodiment, the composition is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compositions that are known to be deleterious to polypeptides.

Generally, the formulations are prepared by contacting the compounds of the invention uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes. Suitable formulations, known in the art, can be found in Remington's Pharmaceutical Sciences (latest edition), Mack Publishing Company, Easton, Pa.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG.

The compositions are typically formulated in such vehicles at a concentration of about 0.01 μg/ml to 100 mg/ml, preferably 0.01 μg/ml to 10 mg/ml, at a pH of about 3 to 8. It will be understood that the use of certain of the foregoing excipients, carriers, or stabilizers will result in the formation of salts.

Compositions to be used for therapeutic administration must be sterile. Sterility is readily accomplished by filtration through sterile filtration membranes (e.g., 0.2 micron membranes). Therapeutic compositions generally are placed into a container having a sterile access port, for example, an intravenous solution bag or vial having a stopper pierceable by a hypodermic injection needle.

The compounds of the invention ordinarily will be stored in unit or multi-dose containers, for example, sealed ampules or vials, as an aqueous solution or as a lyophilized formulation for reconstitution. As an example of a lyophilized formulation, 10-ml vials are filled with 5 ml of sterile-filtered 1% (w/v) aqueous solution, and the resulting mixture is lyophilized. The infusion solution is prepared by reconstituting the lyophilized composition using bacteriostatic Water-for-Injection.

Dosaging may also be arranged in a patient specific manner to provide a predetermined concentration of activity in the blood, as determined by an RIA technique, for instance. Thus patient dosaging may be adjusted to achieve regular on-going trough blood levels, as measured by RIA, on the order of from 50 to 1000 ng/ml, preferably 150 to 500 ng/ml.

The compounds of the invention are useful for administration to any animal, preferably a mammal (such as apes, cows, horses, pigs, boars, sheep, rodents, goats, dogs, cats, chickens, monkeys, rabbits, ferrets, whales, and dolphins), and more preferably a human.

The invention also provides a pharmaceutical pack or kit comprising one or more containers filled with one or more of the ingredients of the pharmaceutical compositions of the invention. Associated with such containers can be a notice in the form prescribed by a governmental agency regulating the manufacture, use or sale of pharmaceuticals or biological products, which notice reflects approval by the agency of manufacture, use or sale for human administration. In addition, the compositions of the present invention may be employed in conjunction with other therapeutic compositions.

Other therapeutic compositions useful for administration along with a compound of the present invention include cytotoxic drugs, particularly those which are used for cancer therapy. Such drugs include, in general, alkylating agents, anti-proliferative agents, tubulin binding agents and the like. Preferred classes of cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of those classes include, for example, adriamycin, caminomycin, daunorubicin, aminopterin, methotrexate, methopterin, dichloromethotrexate, mitomycin C, porfiromycin, 5-fluorouracil, 6-mercaptopurine, cytosine arabinoside, podophyllotoxin, or podophyllotoxin derivatives such as etoposide or etoposide phosphate, melphalan, vinblastine, vincristine, leurosidine, vindesine, leurosine and the like. As noted previously, one skilled in the art may make chemical modifications to the desired compound in order to make reactions of that compound more convenient for purposes of preparing conjugates of the invention.

The compounds of the invention can be used to treat tumor-bearing animals, including humans, to generate an immune response against tumor cells. The generation of an adequate and appropriate immune response leads to tumor regression in vivo. Such “vaccines” can be used either alone or in combination with other therapeutic regimens, including but not limited to chemotherapy, radiation therapy, surgery, bone marrow transplantation, etc. for the treatment of tumors. For example, surgical or radiation techniques could be used to debulk the tumor mass, after which, the vaccine formulations of the invention can be administered to ensure the regression and prevent the progression of remaining tumor masses or micrometastases in the body. Alternatively, administration of the “vaccine” can precede such surgical, radiation or chemotherapeutic treatment.

Alternatively, the recombinant viruses of the invention can be used to immunize or “vaccinate” tumor-free subjects to prevent tumor formation. With the advent of genetic testing, it is now possible to predict a subject's predisposition for certain cancers. Such subjects, therefore, may be immunized using a compound comprising one or more antigenic peptides derived from tumors.

Suitable preparations of such vaccines include injectables, either as liquid solutions or suspensions; solid forms suitable for solution in, suspension in, liquid prior to injection, may also be prepared. The preparation may also be emulsified, or the polypeptides encapsulated in liposomes. The active immunogenic ingredients are often mixed with excipients which are pharmaceutically acceptable and compatible with the active ingredient. Suitable excipients are, for example, water, saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants which enhance the effectiveness of the vaccine.

Examples of adjuvants which may be effective, include, but are not limited to: aluminum hydroxide, N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine, GM-CSF, QS-21 (investigational drug, Progenics Pharmaceuticals,Inc.), DETOX (investigational drug, Ribi Pharmaceuticals), BCG, and CpG rich oligonucleotides.

The composition, if desired, can also contain minor amounts of wetting or emulsifying agents, or pH buffering agents. The composition can be a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

Generally, the ingredients are supplied either separately or mixed together in unit dosage form, for example, as a dry lyophilized powder or water free concentrate in a hermetically sealed container such as an ampoule or sachette indicating the quantity of active agent. Where the composition is administered by injection, an ampoule of sterile diluent can be provided so that the ingredients may be mixed prior to administration.

In an alternate embodiment, compounds of the present invention may be used in adoptive immunotherapeutic methods for the activation of T lymphocytes that are histocompatible with the patient. (for methods of adoptive immunotherapy, see, e.g., Rosenberg, U.S. Pat. No. 4,690,915, issued Sep. 1, 1987; Zarling, et al., U.S. Pat. No. 5,081,029, issued Jan. 14, 1992). Such T lymphocytes may be isolated from the patient or a histocompatible donor. The T lymphocytes are activated in vitro by exposure to the compound of the invention. Activated T lymphocytes are expanded and inoculated into the patient in order to transfer T cell immunity directed against the particular antigenic peptide or peptides.

The compounds of the present invention may be administered along with other compounds which modulate an immune response, for example, cytokines.

The term “cytokine” refers to polypeptides, including, but not limited to, interleukins (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, and IL-18), α interferons (e.g., IFNα), ω interferon (IFNω), β interferons (e.g., IFNβ), γ interferons (e.g., IFNγ), τ interferon (IFNτ), colony stimulating factors (CSFs, e.g., CSF-1, CSF-2, and CSF-3), granulocyte-macrophage colony stimulating factor (GMCSF), transforming growth factor (TGF, e.g.,. TGFα and TGFβ), and insulin-like growth factors (IGFs, e.g., IGF-I and IGF-II).

The compounds of the invention may also be employed in accordance with the present invention by expression of such compounds, especially MHC-peptide-antibody fusion compounds, in vivo, which is often referred to as “gene therapy.”

DNA that encodes a compound of this invention that is a direct fusion of antibody and MHC molecules may be introduced directly into cells by transfection or infection with a suitable vector so as to give rise to synthesis and secretion of that compound by the successfully transfected or infected cells. However, since compounds of this invention require assembly of peptide:MHC complexes and the desired peptides may not be present at high concentration in normal body cells, expression of compounds of the invention through DNA transfection or infection may require that DNA encoding the desired peptide be simultaneously introduced into the cell. This can be accomplished by cotransfection with separate DNA vector constructs or by co-expression in the same vector. In a preferred embodiment two constructs are prepared, an immunoglobulin-MHC class II alpha chain fusion and a specific peptide-MHC class II beta chain fusion (Kozono, H. et al., Nature 369:151 (1994); Zhu, X. et al., Eur. J. Immunol. 27:1933-41 (1997); Rhode, P. R. et al., J. Immunol. 157:4885-91 (1996)). Folding of the linked peptide into the peptide binding of the assembled MHC class II alpha and beta chains will result in a selected antibody specificity linked to a homogeneous population of peptide:MHC complexes. Although single-chain MHC class I fusion proteins have been constructed that incorporate β₂-microblogulin (Toshitani, K. et al., Proc. Nat. Acad. Sci. USA 93:236-40 (1996); Lee, L. et al., Human Immunol. 49:28-37 (1996); Lone, Y. C. et al., J. Immunotherapy 21:283-94 (1998)), efforts to construct single-chain peptide-MHC class I-β₂-microblogulin have been less successful (Sylvester-Hvid, C. et al., Scand. J. Immunol. 50:355-62 (1999). A strategy that could be adopted for this purpose is to split the class I coding sequence between an α2-α3 and a β₂-microblogulin-α1 DNA fragments. Because of the extensive structural similarities between class I and class II molecules, it is expected that the protein fragments would behave very much like MHC class II alpha and beta chains and that they would assemble into functional equivalents of peptide binding class I molecules. Such fragments could then be assembled into compounds of the invention in the same fashion described above for MHC class II based compounds.

Thus, for example, cells from a patient may be engineered with a polynucleotide (DNA or RNA) encoding a compound of the invention ex vivo, with the engineered cells then being provided to a patient to be treated with the compounds. Such methods are well-known in the art. For example, cells may be engineered by procedures known in the art by use of a retroviral particle containing RNA encoding a compound of the present invention.

Similarly, cells may be engineered in vivo for expression of a compound in vivo by, for example, procedures known in the art. As known in the art, a producer cell for producing a retroviral particle containing RNA encoding the compound of the present invention may be administered to a patient for engineering cells in vivo and expression of the polypeptide in vivo. These and other methods for administering a polypeptide of the present invention by such method should be apparent to those skilled in the art from the teachings of the present invention. For example, the expression vehicle for engineering cells may be other than a retrovirus, for example, an adenovirus which may be used to engineer cells in vivo after combination with a suitable delivery vehicle. Examples of other delivery vehicles include an HSV-based vector system, adeno-associated virus vectors, pox viruses, and inert vehicles, for example, dextran coated ferrite particles.

Retroviruses from which the retroviral plasmid vectors hereinabove mentioned may be derived include, but are not limited to, lentiviruses, Moloney Murine Leukemia virus, spleen necrosis virus, retroviruses such as Rous Sarcoma Virus, Harvey Sarcoma virus, avian leukosis virus, gibbon ape leukemia virus, human immunodeficiency virus, adenovirus, Myeloproliferative Sarcoma Virus, and mammary tumor virus. In one embodiment, the retroviral plasmid vector is derived from Moloney Murine Leukemia Virus.

The nucleic acid sequence encoding the compound of the present invention is under the control of a suitable promoter. Suitable promoters which may be employed include, but are not limited to, adenoviral promoters, such as the adenoviral major late promoter; or heterologous promoters, such as cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs (including the modified retroviral LTRs hereinabove described); the β-actin promoter; and human growth hormone promoters.

The retroviral plasmid vector is employed to transduce packaging cell lines to form producer cell lines. Examples of packaging cell lines which may be transfected include, but are not limited to, the PE501, PA317, ψ-2, ψ-AM, PA12, T19-14x, VT-19-17-H2, ψCRE, ψCRIP, GP+E-86, GP+envAm12, and DAN cell lines as described in Miller, Human Gene Therapy 1:5-14 (1990), which is incorporated herein by reference in its entirety. The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line generates infectious retroviral vector particles which include the nucleic acid sequence(s) encoding the polypeptides. Such retroviral vector particles then may be employed, to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

In certain embodiments, the polynucleotide constructs may be delivered as naked polynucleotides. By “naked” polynucleotides is meant that the polynucleotides are free from any delivery vehicle that acts to assist, promote, or facilitate entry into the cell, including viral sequences, viral particles, liposome formulation, lipofectin, precipitating agents and the like. Such methods are well known in the art and described, for example, in U.S. Pat. Nos. 5,593,972, 5,589,466, and 5,580,859.

The naked polynucleotides used in the invention can be those which do not integrate into the genome of the host cell. These may be non-replicating sequences, or specific replicating sequences genetically engineered to lack the genome-integration ability. Alternatively, the naked polynucleotides used in the invention may integrate into the genome of the host cell by, for example, homologous recombination, as discussed below. Preferably, the naked polynucleotide construct is contained in a plasmid. Suitable expression vectors for delivery include, but are not limited to, vectors such as pRSVcat (ATCC 37152), pSVL and MSG (Pharmacia, Uppsala, Sweden), pSV2dhfr (ATCC 37146) and pBC12MI (ATCC 67109). Additional suitable plasmids are discussed in more detail above.

The naked polynucleotides can be administered to any tissue (such as muscle tissue) or organ, as described above. In another embodiment, the naked polynucleotides are administered to the tissue surrounding the tissue of origin. In another embodiment, the naked polynucleotides are administered systemically, through intravenous injection.

For naked polynucleotide injection, an effective dosage amount of polynucleotide will be in the range of from about 0.05 μg/kg body weight to about 50 mg/kg body weight. Preferably, the dosage will be from about 0.005 mg/kg to about 20 mg/kg and more preferably from about 0.05 mg/kg to about 5 mg/kg. The appropriate and effective dosage of the polynucleotide construct can readily be determined by those of ordinary skill in the art and may depend on the condition being treated and the route of administration.

The constructs may also be delivered with delivery vehicles such as viral sequences, viral particles, liposome formulations, lipofectin, precipitating agents, etc. Such methods of delivery are known in the art. For example, the polynucleotide construct can be delivered specifically to hepatocytes through the method of Wu et al., J. Biol. Chem. 264:6985-16987 (1989).

In certain embodiments, the polynucleotide constructs are complexed in a liposome preparation. Liposomal preparations for use in the instant invention include cationic (positively charged), anionic (negatively charged) and neutral preparations. However, cationic liposomes are particularly preferred because a tight charge complex can be formed between the cationic liposome and the polyanionic nucleic acid. Cationic liposomes have been shown to mediate intracellular delivery of plasmid DNA (Felgner et al., Proc. Natl. Acad. Sci. USA (1987) 84:7413-7416); mRNA (Malone et al., Proc. Natl. Acad. Sci. USA (1989) 86:6077-6081); and purified transcription factors (Debs et al., J. Biol. Chem. (1990) 265:10189-10192), in functional form.

Cationic liposomes are readily available. For example, N[1-2,3-dioleyloxy)propyl]-N,N,N-triethylammonium (DOTMA) liposomes are particularly useful and are available under the trademark Lipofectin, from GIBCO BRL, Grand Island, N.Y. (See, also, Felgner et al., Proc. Natl. Acad. Sci. USA (1987) 84:7413-7416). Other commercially available liposomes include transfectace (DDAB/DOPE) and DOTAP/DOPE (Boehringer).

Other cationic liposomes can be prepared from readily available materials using techniques well known in the art. See, e.g. PCT Publication No. WO 90/11092 for a description of the synthesis of DOTAP (1,2-bis(oleoyloxy)-3-(trimethylammonio)propane) liposomes. Preparation of DOTMA liposomes is explained in the literature, see, e.g., P. Felgner et al., Proc. Natl. Acad. Sci. USA 84:7413-7417. Similar methods can be used to prepare liposomes from other cationic lipid materials.

Similarly, anionic and neutral liposomes are readily available, such as from Avanti Polar Lipids (Birmingham, Ala.), or can be easily prepared using readily available materials. Such materials include phosphatidyl, choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

For example, commercially dioleoylphosphatidyl choline (DOPC), dioleoylphosphatidyl glycerol (DOPG), and dioleoylphosphatidyl ethanolamine (DOPE) can be used in various combinations to make conventional liposomes, with or without the addition of cholesterol. Thus, for example, DOPG/DOPC vesicles can be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator equipped with an inverted cup (bath type) probe at the maximum setting while the bath is circulated at 15° C. Alternatively, negatively charged vesicles can be prepared without sonication to produce multilamellar vesicles or by extrusion through nucleopore membranes to produce unilamellar vesicles of discrete size. Other methods are known and available to those of skill in the art.

The liposomes can comprise multilamellar vesicles (MLVs), small unilamellar vesicles (SUVs), or large unilamellar vesicles (LUVs), with SUVs being preferred. The various liposome-nucleic acid complexes are prepared using methods well known in the art. See, e.g., Straubinger et al., Methods of Immunology (1983),101:512-527. For example, MLVs containing nucleic acid can be prepared by depositing a thin film of phospholipid on the walls of a glass tube and subsequently hydrating with a solution of the material to be encapsulated. SUVs are prepared by extended sonication of MLVs to produce a homogeneous population of unilamellar liposomes. The material to be entrapped is added to a suspension of preformed MLVs and then sonicated. When using liposomes containing cationic lipids, the dried lipid film is resuspended in an appropriate solution such as sterile water or an isotonic buffer solution such as 10 mM Tris/NaCl, sonicated, and then the preformed liposomes are mixed directly with the DNA. The liposome and DNA form a very stable complex due to binding of the positively charged liposomes to the cationic DNA. SUVs find use with small nucleic acid fragments. LUVs are prepared by a number of methods, well known in the art. Commonly used methods include Ca²⁺-EDTA chelation (Papahadjopoulos et al., Biochim. Biophys. Acta (1975) 394:483; Wilson et al., Cell (1979) 17:77); ether injection (Deamer, D. and Bangham, A., Biochim. Biophys. Acta (1976) 443:629; Ostro et al., Biochem. Biophys. Res. Commun. (1977) 76:836; Fraley et al., Proc. Natl. Acad. Sci. USA (1979) 76:3348); detergent dialysis (Enoch, H. and Strittmatter, P., Proc. Natl. Acad. Sci. USA (1979) 76:145); and reverse-phase evaporation (REV) (Fraley et al., J. Biol. Chem. (1980)255:10431; Szoka, F. and Papahadjopoulos, D., Proc. Natl. Acad. Sci. USA (1978) 75:145; Schaefer-Ridder et al., Science (1982) 215:166).

Additional examples of useful cationic lipids include dipalmitoyl-phophatidylethanolamine 5-carboxyspen-nylamide (DPPES); 5-carboxyspermylglycine dioctadecylamide (DOGS); dimethyldioctdecyl-ammonium bromide (DDAB); and (±)-N,N-dimethyl-N-[2-(sperminecarboxamido)ethyl]-2,3-bis(dioleyloxy)-1-propaniminium pentahydrochloride (DOSPA). Non-diether cationic lipids, such as DL-1,2-dioleoyl-3-dimethylaminopropyl-p-hydroxyethylammonium (DOR1 diester), 1,2-O-dioleyl-3-dimethylaminopropyl-β-hydroxyethylammonium (DORIE diether), 1-O-oleyl-2-oleoyl-3-dimethylaminopropyl-β-hydroxyethylammonium (DOR1 ester/ether), and their salts promote in vivo gene delivery. Cationic cholesterol derivatives such as, {3β[N-N′,N′-dimethylamino)ethane]-carbomoyl}-cholesterol (DC-Chol), are also useful.

Preferred cationic lipids include: (±)-N-(2-hydroxyethyl)-N,N-dimethyl-2,3-bis(tetradecyloxy)-1-propaniminium bromide; 3,5-(N,N-di-lysyl)diamino-benzoylglycyl-3-(DL-1,2-dioleoyl-dimethylaminopropyl-β-hydroxyethylamine) (DLYS-DABA-GLY-DORI diester); 3,5-(NN-dilysyl)-diaminobenzoyl-3-(DL-1,2-dioleoyl-dimethylaminopropyl-β-hydroxyethylamine) (DLYS-DABA-DORI diester); and 1,2-dioleoyl-sn-glycero-3-phosphoethanolamine. Also preferred is the combinations of the following lipids: (±)-N-(2-hydroxyethyl)-N,N-dimethyl-2,3-bis(tetradecyloxy)-1-propaniminium bromide and 1,2-dioleoyl-sn-glycero-3-phosphoethanolamine; and (±)-N-(2-hydroxyethyl)-N,N-dimethyl-2,3-bis(tetradecyloxy)-1-propaniminium bromide, and 1,2-dioleoyl-sn-glycero-3-phosphoethanolamine in a 1:1 ratio.

The lipid formulations may have a cationic lipid alone, or also include a neutral lipid such as cardiolipin, phosphatidylcholine, phosphatidylethanolamine, dioleoylphosphatylcholine, dioleoylphosphatidyl-ethanolamine, 1,2-dioleoyl-sn-glycero-3-phosphatidylethanolamine (DOPE), sphingomyelin, and mono-, di- or tri-acylglycerol).

Lipid formulations may also have cationic lipid together with a lysophosphatide. The lysophosphatide may have a neutral or a negative head group. Useful lysophosphatides include lysophosphatidylcholine, lysophosphatidyl-ethanolamine, and 1-oleoyl lysophosphatidylcholine. Lysophosphatide lipids are present Other additives, such as cholesterol, fatty acid, ganglioside, glycolipid, neobee, niosome, prostaglandin, sphingolipid, and any other natural or synthetic amphiphiles, can be used. A preferred molar ratio of cationic lipid to neutral lipid in these lipid formulations is from about 9:1 to about 1:9; an equimolar ratio is more preferred in the lipid-containing formulation in a 1:2 ratio of lysolipid to cationic lipid.

Generally, the ratio of DNA to liposomes will be from about 10:1 to about 1:10. Preferably, the ratio will be from about 5:1 to about 1:5. More preferably, the ratio will be about 3:1 to about 1:3. Still more preferably, the ratio will be about 1:1.

U.S. Pat. No. 5,676,954 reports on the injection of genetic material, complexed with cationic liposomes carriers, into mice. U.S. Pat. Nos. 4,897,355, 4,946,787, 5,049,386, 5,459,127, 5,589,466, 5,693,622, 5,580,859, 5,703,055, and international publication no. WO 94/9469 provide cationic lipids for use in transfecting DNA into cells and mammals. U.S. Pat. Nos. 5,589,466, 5,693,622, 5,580,859, 5,703,055, and international publication no. WO 94/9469 provide methods for delivering DNA-cationic lipid complexes to mammals.

In certain other embodiments, cells are engineered, ex vivo or in vivo, with the polynucleotide operably linked to a promoter contained in an adenovirus vector. Adenovirus can be manipulated such that it encodes and expresses the desired gene product, and at the same time is inactivated in terms of its ability to replicate in a normal lytic viral life cycle. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartz, A. R. et al (1974) Am. Rev. Respir. Dis. 109:233-238). Finally, adenovirus mediated gene transfer has been demonstrated in a number of instances including transfer of alpha-1-antitrypsin and CFTR to the lungs of cotton rats (Rosenfeld, M. A. et al. (1991) Science 252:431-434; Rosenfeld et al., (1992) Cell 68:143-155). Furthermore, extensive studies to attempt to establish adenovirus as a causative agent in human cancer were uniformly negative (Green, M. et al. (1979) Proc. Natl. Acad. Sci. USA 76:6606).

Suitable adenoviral vectors useful in the present invention are described, for example, in Kozarsky and Wilson, Curr. Opin. Genet. Devel. 3:499-503 (1993); Rosenfeld et al., Cell 68:143-155 (1992); Engelhardt et al., Human Genet. Ther. 4:759-769 (1993); Yang et al, Nature Genet. 7:362-369 (1994); Wilson et al., Nature 365:691-692(1993); and U.S. Pat. No. 5,652,224, which are herein incorporated by reference. For example, the adenovirus vector Ad2 is useful and can be grown in human 293 cells. These cells contain the E1 region of adenovirus and constitutively express E1a and E1b, which complement the defective adenoviruses by providing the products of the genes deleted from the vector. In addition to Ad2, other varieties of adenovirus (e.g., Ad3, Ad5, and Ad7) are also useful in the present invention.

Preferably, the adenoviruses used in the present invention are replication deficient. Replication deficient adenoviruses require the aid of a helper virus and/or packaging cell line to form infectious particles. The resulting virus is capable of infecting cells and can express a polynucleotide of interest which is operably linked to a promoter, for example, the polynucleotide of the present invention, but cannot replicate in most cells. Replication deficient adenoviruses may be deleted in one or more of all or a portion of the following genes: E1a, E1b, E3, E4, E2a, or L1 through L5.

In certain other embodiments, the cells are engineered, ex vivo or in vivo, using an adeno-associated virus (AAV). AAVs are naturally occurring defective viruses that require helper viruses to produce infectious particles (Muzyczka, N., Curr. Topics in Microbiol. Immunol. 158:97 (1992)). It is also one of the few viruses that may integrate its DNA into non-dividing cells. Vectors containing as little as 300 base pairs of AAV can be packaged and can integrate, but space for exogenous DNA is limited to about 4.5 kb. Methods for producing and using such AAVs are known in the art. See, for example, U.S. Pat. Nos. 5,139,941, 5,173,414, 5,354,678, 5,436,146, 5,474,935, 5,478,745, and 5,589,377.

For example, an appropriate AAV vector for use in the present invention will include all the sequences necessary for DNA replication, encapsidation, and host cell integration. The polynucleotide construct is inserted into the AAV vector using standard cloning methods, such as those found in Sambrook et al., Molecular Cloning. A Laboratory Manual, Cold Spring Harbor Press (1989). The recombinant AAV vector is then transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the polynucleotide construct. These viral particles are then used to transduce eukaryotic cells, either ex vivo or in vivo. The transduced cells will contain the polynucleotide construct integrated into its genome, and will express the molecule of interest.

Any mode of administration of any of the above-described polynucleotides constructs can be used so long as the mode results in the expression of one or more molecules in an amount sufficient to provide a therapeutic effect. This includes direct needle injection, systemic injection, catheter infusion, biolistic injectors, particle accelerators (i.e., “gene guns”), gelfoam sponge depots, other commercially available depot materials, osmotic pumps (e.g., Alza minipumps), oral or suppositorial solid (tablet or pill) pharmaceutical formulations, and decanting or topical applications. For example, direct injection of naked calcium phosphate-precipitated plasmid into rat liver and rat spleen or a protein-coated plasmid into the portal vein has resulted in gene expression of the foreign gene in the rat livers (Kaneda et al., Science 243:375 (1989)).

A preferred method of local administration is by direct injection. Preferably, a recombinant molecule of the present invention complexed with a delivery vehicle is administered by direct injection into or locally within the area of the liver. Administration of a composition locally within the area of the liver refers to injecting the composition centimeters and preferably, millimeters within the liver.

Another method of local administration is to contact a polynucleotide-promoter construct of the present invention in or around a surgical wound. For example, a patient can undergo surgery and the polynucleotide construct can be coated on the surface of tissue inside the wound or the construct can be injected into areas of tissue inside the wound.

Therapeutic compositions useful in systemic administration, include recombinant molecules of the present invention complexed to a targeted delivery vehicle of the present invention. Suitable delivery vehicles for use with systemic administration comprise liposomes comprising ligands for targeting the vehicle to a particular site, for example, ligands for targeting the vehicle to a tissue of interest. Targeting vehicles for other tissues and organs are well known to skilled artisans.

Preferred methods of systemic administration, include intravenous injection, aerosol, oral and percutaneous (topical) delivery. Intravenous injections can be performed using methods standard in the art. Aerosol delivery can also be performed using methods standard in the art (see, for example, Stribling et al., Proc. Natl. Acad. Sci. USA 189: 11277-11281, 1992, which is incorporated herein by reference). Oral delivery can be performed by complexing a polynucleotide construct of the present invention to a carrier capable of withstanding degradation by digestive enzymes in the gut of an animal. Examples of such carriers, include plastic capsules or tablets, such as those known in the art. Topical delivery can be performed by mixing a polynucleotide construct of the present invention with a lipophilic reagent (e.g., DMSO) that is capable of passing into the skin.

Determining an effective amount of substance to be delivered can depend upon a number of factors including, for example, the chemical structure and biological activity of the substance, the age and weight of the animal, the precise condition requiring treatment and its severity, and the route of administration. The frequency of treatments depends upon a number of factors, such as the amount of polynucleotide constructs administered per dose, as well as the health and history of the subject. The precise amount, number of doses, and timing of doses will be determined by the attending physician or veterinarian.

Direct administration of a DNA construct coding for a compound of the invention can be suitably accomplished for expression of the fusion compound within cells of the subject. Also, rather than directly administering nucleic acids coding for a compound of the invention to a subject, host compatible cells into which such nucleic acids have been introduced may be administered to the subject. Upon administration to a subject, such engineered cells can then express in vivo the compound of the invention. Such engineered cells can be administered to a subject to induce an immune response or alternatively to suppress an immune response, as disclosed herein.

A treatment method for suppression of an immune response provides for administration of a compound of the invention in which the peptide is a TCR antagonist or partial agonist. See Sette et al., Ann. Rev. Immunol. 12:413-431 (1994)). Peptides that are TCR antagonists or partial agonists can be readily identified and selected by the in vitro protocols identified above. A compound of the invention that contains a peptide that is a TCR antagonist or partial agonist is particularly preferred for treatment of allergies and autoimmune diseases.

Immunosuppressive therapies of the invention also may be used in combination as well as with other known immunosuppressive agents such as anti-inflammatory drugs to provide a more effective treatment of a T cell-mediated disorder. For example, other immunosuppressive agents useful in conjunction with the compounds of the invention include anti-inflammatory agents such as corticosteroids and nonsteroidal drugs.

The invention also provides methods for invoking an immune response in a mammal such as a human, including vaccinating a mammal with a compound or composition described herein.

The compounds of the invention are useful for raising an immune response and treating hyperproliferative disorders. Examples of hyperproliferative disorders that can be treated by the compounds of the invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

Similarly, other hyperproliferative disorders can also be treated by the compounds of the invention. Examples of such hyperproliferative disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

The compounds of the present invention are also useful for raising an immune response against infectious agents. Viruses are one example of an infectious agent that can cause disease or symptoms that can be treated by the compounds of the invention. Examples of viruses, include, but are not limited to the following DNA and RNA viral families: Arbovirus, Adenoviridae, Arenaviridae, Arterivirus, Bimaviridae, Bunyaviridae, Caliciviridae, Circoviridae, Coronaviridae, Flaviviridae, Hepadnaviridae (hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza), Papovaviridae, Parvoviridae, Picomaviridae, Poxyiridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, measles, mumps, parainfluenza, rabies, the common cold, Polio, leukemia, Rubella, sexually transmitted diseases, skin diseases (e.g., Kaposi's, warts), and viremia.

Similarly, bacterial or fungal agents that can cause disease or symptoms and that can be treated by the compounds of the invention include, but are not limited to, the following Gram-Negative and Gram-positive bacterial families and fungi: Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeria, Mycoplasmatales, Neisseriaceae (e.g., Acinetobacter, Gonorrhea, Menigococcal), Pasteurellacea Infections (e.g., Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, wound infections.

Moreover, parasitic agents causing disease or symptoms that can be treated by the compounds of the invention include, but are not limited to, the following families: amebiasis, babesiosis, coccidiosis, cryptosporidiosis, dientamoebiasis, dourine, ectoparasitic, giardiasis, helminthiasis, leishmaniasis, theileriasis, toxoplasmosis, trypanosomiasis, and trichomonas.

Additionally, the compounds of the invention are useful for treating autoimmune diseases. An autoimmune disease is characterized by the attack by the immune system on the tissues of the victim. In autoimmune diseases, the recognition of tissues as “self” apparently does not occur, and the tissue of the afflicted subject is treated as an invader—i.e., the immune system sets about destroying this presumed foreign target. The compounds of the present invention are therefor useful for treating autoimmune diseases by desensitizing the immune system to these self antigens by provided a TCR signal to T cells without a costimulatory signal or with an inhibitory signal.

Examples of autoimmune diseases which may be treated using the compounds of the present invention include, but are not limited to Addison's Disease, hemolytic anemia, antiphospholipid syndrome, rheumatoid arthritis, dermatitis, allergic encephalomyelitis, glomerulonephritis, Goodpasture's Syndrome, Graves' Disease, multiple sclerosis, myasthenia gravis, neuritis, ophthalmia, bullous pemphigoid, pemphigus, polyendocrinopathies, purpura, Reiter's Disease, Stiff-Man Syndrome, autoimmune thyroiditis, systemic lupus erythematosus, autoimmune pulmonary inflammation, Guillain-Barre Syndrome, insulin dependent diabetes mellitis, autoimmune inflammatory eye disease, autoimmune hemolysis, psoriasis, juvenile diabetes, primary idiopathic myxedema, autoimmune asthma, scleroderma, chronic hepatitis, hypogonadism, pernicious anemia, vitiligo, alopecia areata, Coeliac disease, autoimmune enteropathy syndrome, idiopathic thrombocytic purpura, acquired splenic atrophy, idiopathic diabetes insipidus, infertility due to antispermatazoan antibodies, sudden hearing loss, sensoneural hearing loss, polymyositis, autoimmune demyelinating diseases, traverse myelitis, ataxic sclerosis, progressive systemic sclerosis, dermatomyositis, polyarteritis nodosa, idiopathic facial paralysis, cryoglobulinemia, inflammatory bowel diseases, Hashimoto's disease, adrenalitis, hypoparathyroidism, and ulcerative colitis.

Similarly, allergic reactions and conditions, such as asthma (particularly allergic asthma) or other respiratory problems, may also be treated by compounds of the invention. Moreover, the compounds of the invention can be used to treat anaphylaxis, hypersensitivity to an antigenic molecule, or blood group incompatibility.

The compounds of the invention may also be used to treat and/or prevent organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of the compounds of the invention that inhibit an immune response may be an effective therapy in preventing organ rejection or GVHD.

The compounds of the invention which can inhibit an immune response are also useful for treating and/or preventing atherosclerosis; olitis; regional enteritis; adult respiratory distress syndrome; local manifestations of drug reactions, such as dermatitis, etc.; inflammation-associated or allergic reaction patterns of the skin; atopic dermatitis and infantile eczema; contact dermatitis; psoriasis; lichen planus; allergic enteropathies; allergic rhinitis; bronchial asthma; hypersensitivity or destructive responses to infectious agents; poststreptococcal diseases, e.g. cardiac manifestations of rheumatic fever, and the like.

Further, the compounds of the invention can be used as a male or female contraceptive. For example, a compound of the invention which is useful as a male contraceptive comprises as the antigenic peptide a peptide derived from PH30 beta chain sperm surface protein. See U.S. Pat. No. 5,935,578. A compound of the invention which is useful as a female contraceptive may comprise as the antigenic peptide a peptide derived from the human ZP2 or the human ZP3 protein. See U.S. Pat. No. 5,916,768.

A preferred method of delivering compounds of the invention is to administer them directly (iv, im, id, po) in the absence or presence of adjuvants such as oil and water emulsions, alum, CpG oligonucleotides, or cytokines such as GM-CSF. Another approach is to isolate patient PBL, purify PBMC and generate dendritic cells by a modification of the above protocol employing culture medium approved for clinical use such as X-VIVO or AIM-V and immunomagnetic bead separation of monocytes and lymphocytes rather than sheep erythrocyte rosetting (Romani, N., et al. J. Immunol. Methods. 196:137-151 (1996)). These cells can be pulsed in vitro with the compounds of the invention and then administered to the patient. This approach circumvents potential in vivo clearance of the compounds of the invention in the circulation, allows utilization of higher concentrations of the compound in vitro than would be possible or allowed in vivo, and ensures effective delivery of dendritic cells armed and ready to stimulate a primary T cell response. A secondary injection of pre-loaded DC or compound alone may be employed to boost the immune response. The magnitude of T cell responses induced is determined in vitro by a variety of assays for antigen-specific T cell activation as described herein or by staining with tetrameric complexes of the same peptide:MHC ligand as described herein.

Having generally described the invention, the same will be more readily understood by reference to the following examples, which are provided by way of illustration and are not intended as limiting.

### EXAMPLES

### Example 1

### Construction of Human IgG3-Avidin Fusion Antibodies

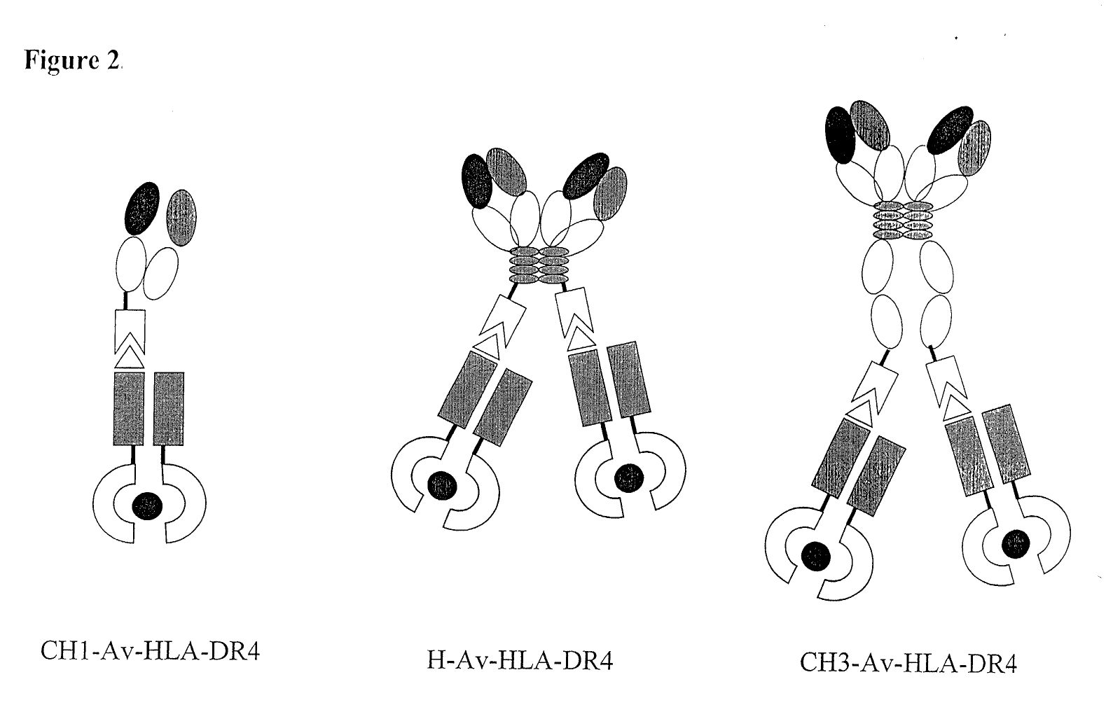

The construction of a human IgG3-Avidin fusion antibody with specificity for the hapten dansyl has been previously described (S. U. Shin et al., J. Immunology 158: 4797-4804 (1997)). The objective of this work is to replace the portion of this molecule that provides binding specificity for dansyl (V_{H} and V_{L} domains) with the homologous domains (V_{H} and V_{L}) from antibodies that are specific for cell surface molecules. These resulting antibodies will retain the ability to bind biotinylated tetrameric MHC complexes, and will allow for the targeting of these tetramers to the cell type of interest (for example, professional antigen-presenting cells, T cells, tumor cells, epithelial cells, or fibroblasts) (FIGS. 1, 2).

Monoclonal antibodies specific for DC specific molecules such as CD83, CMRF-44, and CMRF-56 (Table 5); for T cell specific molecules such as CD28, CTLA-4, and CD25 (Table 5); and for tumor specific molecules such as Mucl, and Her2/neu (Table 6) have been isolated. To construct compounds that will target MHC tetramers to cells expressing these molecules, the genes that encode the V_{H} and V_{L} domains of antibodies specific for these molecules are isolated from the hybridoma cells that produce the specific antibodies. The heavy and light chain variable regions of the anti-dansyl avidin antibody are then replaced with these variable region genes.

Hybridoma cells secreting antibodies specific for the cell markers of interest are used as the source of the variable region genes. Messenger RNA is isolated from these hybridomas, converted into double stranded cDNA, ligated into a plasmid vector, and transformed into bacteria in order to generate a cDNA library. This cDNA library is screened using a probe derived from the Constant (C) region of the Ig Heavy chain, and separately with a probe derived from the C region of the Ig light chain, using the ClonCapture cDNA Selection System (Clontech, Palo Alto, Calif.). Clones recombinant for the Ig cDNA are sequenced in order to determine the sequence of the heavy and light chain variable region genes. Once these full-length cDNAs (containing the coding region for the entire Ig) have been isolated, the next step is to replace the variable region genes of the anti-dansyl antibody with these newly isolated variable region genes.

The cDNA containing the heavy chain LVDJ domain of the antibody is modified by PCR to include an NheI site at the 5′ end, and an intron splice donor (SD) sequence (GTAAGT) and XbaI site at its 3′ end. The sequence of the sense primer is 5′ AAT GCT AGC N₍₁₂₋₂₀₎ (SEQ ID NO:1) and the antisense primer is 5′ ATT TCT AGA ACT TAC N₍₁₂₋₂₀₎ (SEQ ID NO:2). The unknown nucleotides (N) in the primers are designed according to the sequence of the Leader sequence (including the ATG start codon) (sense primer), or to the Joining Segment (J) (antisense primer). Following digestion with NheI and XbaI, this LVDJ SD PCR product is inserted into the NheI and XbaI sites of expression vector pcDNA3.1/Hygro(−)(Invitrogen), creating pcDNA3.1/Hygro/IgVH. The gene encoding the heavy chain of the anti-Dansyl IgG3-Avidin antibody is contained in plasmids pAG3520, pAG3513, and pAG3517. pAG3520 contains CH1-H-CH2-CH3; pAG3513 contains CH1-H; and pAG3517 contains CH1. The Ig-avidin portion of this molecule is excised from each vector by digestion with Sal I and Bam HI. This IgG3-avidin cassette is inserted into the XhoI/BamHI sites of pcDNA3.1/Hygro/IgVH (SalI and XhoI leave complementary overhangs), creating pcDNA3.1/IgVH/X, wherein X indicates CH1-Avidin, H-Avidin, or CH3-Avidin. Following transcription, the splice donor sequence at the 3′ end of the LVDJ is spliced in frame with the splice acceptor sequence at the 5′ end of the CH1 exon. The spliced mRNA encodes an human IgG3-avidin fusion protein.

The cDNA containing the light chain LVJ domain of the antibody is modified by PCR to include an NheI site at the 5′ end, and an intron splice donor (SD) sequence (GTAAGT) and XbaI site at its 3′ end. The sequence of the sense primer is 5′ AAT GCT AGC N₍₁₂₋₂₀₎ and the antisense primer is 5′ ATT TCT AGA ACT TAC N₍₁₂₋₂₀₎ (SEQ ID NO:2). The unknown nucleotides (N) in the primers are designed according to the sequence of the leader sequence (including the ATG start codon) (sense primer), or to the Joining Segment (J) (antisense primer). Following digestion with NheI and XbaI, this LVJ SD PCR product is inserted into the NheI and XbaI sites of expression vector pcDNA3.1/Neo(−)(Invitrogen), creating pcDNA3.1/Neo/IgVL. The gene encoding the constant (C) Domain of the human Kappa Ig is available in vector pCN101. The coding region of the human IgK C gene is isolated from this vector by PCR with a sense primer containing an XbaI site (5′ AATTCTAGAGTCTGTCCCTAACATGCCC (SEQ ID NO:3)), and a KpnI site on the antisense primer (5′ AAAGGTACCT GGAACTGAGGAGCAGGTG (SEQ ID NO:4)).

Following digestion with XbaI and KpnI, the IgCκ coding region is inserted into the XbaI and KpnI sites of pcDNA3.1/neo/IgVL, resulting in pcDNA3.1/neo/IgVL/K. Following transcription, the splice donor sequence at the 3′ end of the LVJ is spliced in frame with the splice acceptor sequence at the 5′ end of the K exon. The spliced mRNA encodes a human kappa light chain variable region fusion protein of an antibody.

Transfection, for example, of both pcDNA3.1/IgVH/IgG3-Avidin and pcDNA3.1/neo/IgVL/K into a non-Ig secreting B cell line generates a hybridoma that secretes antibody-avidin fusion molecules that are specific for the desired molecule. Biotinylated MHC class I or MHC class II molecules can be assembled into polymeric complexes on these antibody-avidin fusion proteins in exactly the same way previously described for assembly on free streptavidin (Altman, J. et al., Science 274:94-96 (1996); Boniface, J. J. et al., Immunity 9:459-66 (1998)).

### Example 2

### Construction of IgG3-HLA Fusion Proteins

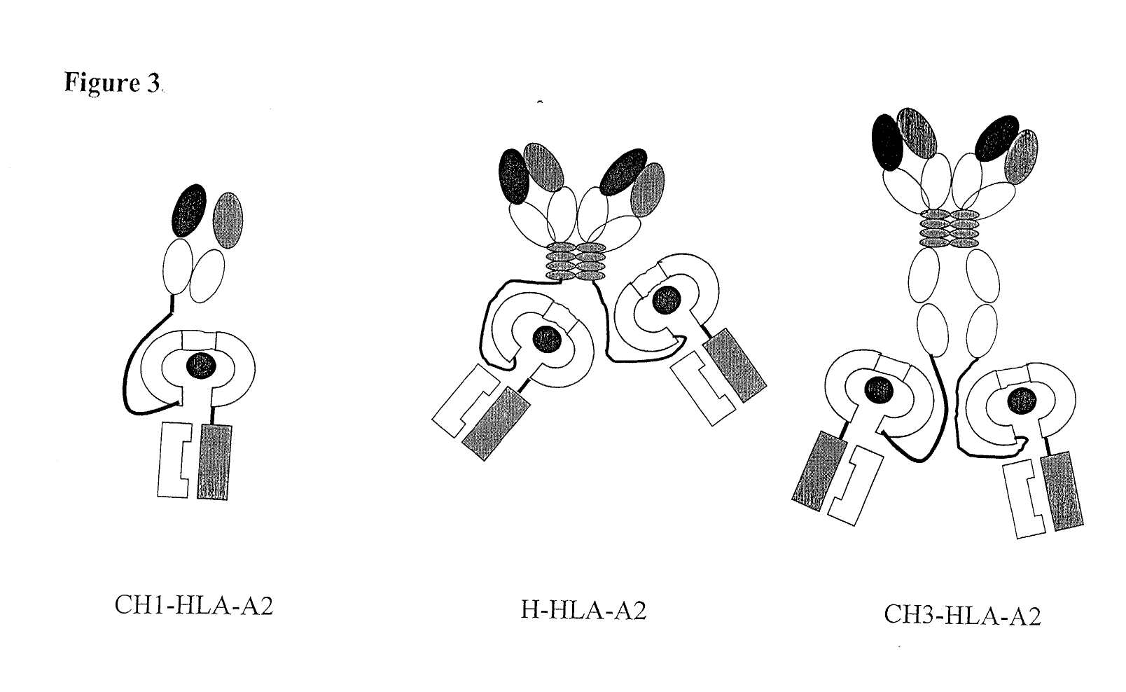

A strategy for construction of the IgG3-HLA-A2 fusion proteins depicted in FIG. 3 is described. Modifications of this strategy required for construction of other IgG3-HLA fusion proteins will be evident to those skilled in the art. The cDNA encoding the extracellular region of HLA-A2 (nucleotides 73-885 (GenBank Accessionno. M84379)) (Garboczi, D. N. et al., Proc. Natl. Acad. Sci. USA 89: 3429-3433 (1992); Altman, J. D. et al., Science 274: 94-96 (1996)) is amplified by PCR using a sense primer containing an NheI site (5′ AATGCTAGCGGCTCTCACTCCATG (SEQ ID NO:5)) and an antisense primer containing a stop codon and an EcoRI site (5′ ATTGAATTCTTAGG TGAGGGGCTTGGG (SEQ ID NO:6)). Following digestion of the HLA-A2 PCR product with Nhe I, an adapter is ligated onto this PCR product. This HLA Adapter contains a coding sequence for: 5′ PvuII site GG (Gly₄Ser)₂ (SEQ ID NO:7) NheI sticky end 3′ and is generated by annealing the single stranded oligos “HLA Adapter Sense” (5′ TTTCAGCTGGGGGCGGCGG CGGCTCTGGCGGC GGCGGCTCTG (SEQ ID NO:8)) and “HLA Adapter Antisense” (5′CAGAGCC GCCGCCGCCAGAGCCGCCGCCGCCCCCAGCTGAAA (SEQ ID NO:9)).

Following digestion with PvuI and EcoRI, the adapter modified HLA-A2 is cloned in frame with the CH3 (pAT3462), H (pAT4401), or CH1 (pAT3452) exons of human IgG3. Insertion into pAT3462 is at the SspI and EcoRI sites, into pAT4401 at PvuII and EcoRI, and into pAT3452 at SnaBI and EcoRI (SspI, PvuII, and SnaBI leave blunt ends). In all 3 constructs, the Spacer/HLA-A2 is in frame with the Ig. The exons containing CH3-HLA-A2, H-HLA-A2, and CH1-HLA-A2 can be excised with SalI and BamHI, and inserted into the XhoI and BamHI sites of pcDNA3.1/Hygro/IgVH, resulting in pcDNA3.1/Hygro/IgVH/X, wherein X is IgG3-HLA-A2, H-HLA-A2, or CH1-HLA-A2. These constructs encode antibody-HLA fusion proteins with specificity for a cell surface marker.

### Example 3

### Construction of IgG3 Antibody-HLA-DR4 Fusion Proteins

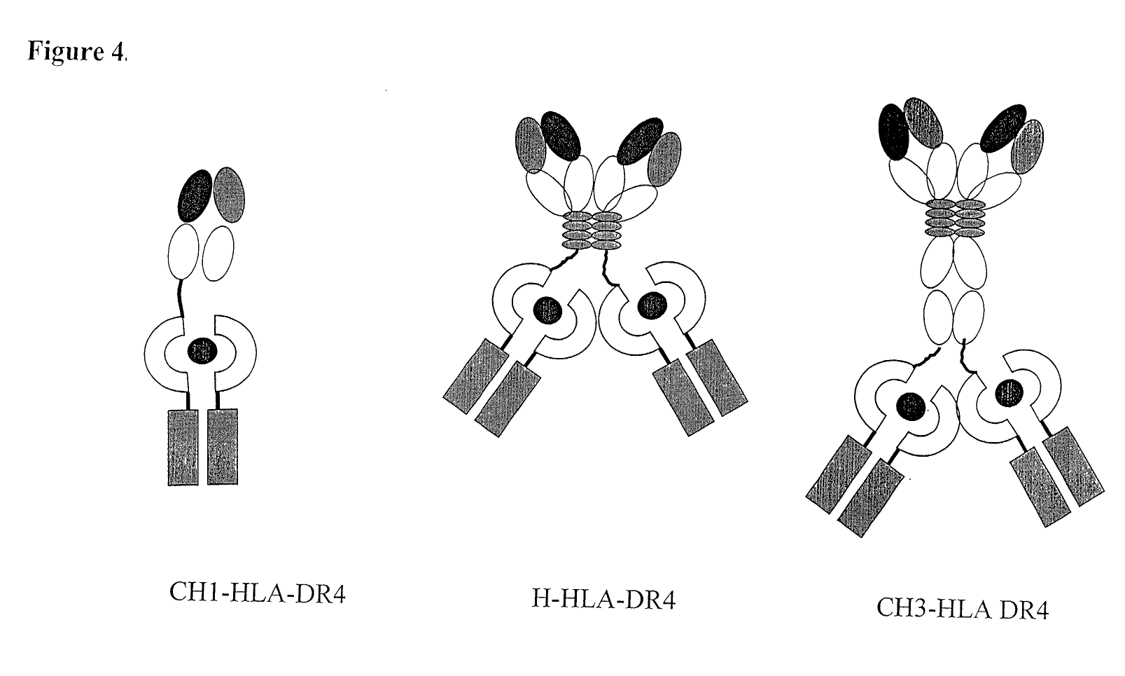

A strategy for construction of an antibody-HLA-DR4 fusion protein depicted in FIG. 4 is described. Modifications of this strategy required for construction of other IgG3-HLA class II fusion proteins will be evident to those skilled in the art. This example describes the construction of molecules containing an Ig heavy chain-HLA-DR4 B chain fusion protein. Purified HLA class II A chain protein will be mixed with this antibody-HLA B chain protein and allowed to fold in vitro in the presence of the desired peptide. This will generate antibody molecules with fully assembled HLA Class II molecules. The reciprocal fusion proteins (Ig heavy chain-HLA-DR4 A chain, free HLA-DR4 B chain) can be constructed in similar fashion.

The extracellular region of the HLA-DR4 B chain (exons 2-3) is PCR amplified using a sense primer containing an NheI site (5 AAAGCTAGCGGGG ACACCCGACCA (SEQ ID NO:10)) and an antisense primer containing an EcoRI site and a stop codon (5′ AAAGAATTCATTCATCTTGCTCTGTGCA GATT (SEQ ID NO: 11)). Following digestion of the HLA-DR4 B PCR product with Nhe I, the HLA Adapter is ligated onto this PCR product. This molecule is then digested with PvuII and EcoRI and inserted into the SspI and EcoRI sites of pAT4401, the PvuI and EcoRI sites of pAT3462, or the SnaBI and EcoRI sites of pAT3452, generating CH3-HLA DR4 B, H-HLA DR4 B, and CH1-HLA DR4 B respectively. The Ig-HLA DR4 B is excised with SalI and BamHI and inserted into the XhoI and BamHI sites of pcDNA3.1/Hygro/IgVH, generating pcDNA3.1/Hygro/IgVH/X-HLA DR4. Following transfection and expression, the Antibody-HLA-DR4 B molecule is purified and incubated with purified HLA DR4 A chain and peptide. This will generate antibody molecules containing fully assembled HLA DR4 molecules.

In an alternative strategy, the cDNA for the extracellular region of the HLA-DR4 A chain is fused to the C terminus of the Light chain kappa constant region gene. This fusion gene is coexpressed with the Ig-HLA-DR4 B fusion gene described above, allowing for the in vivo assembly of the Antibody-HLA-DR4 Class II molecules. The first step in making this construct is to PCR amplify the kappa C region using a sense primer with an XbaI site (5′ AATTCTAGAGAACTGTGGCTGCACCAT (SEQ ID NO:12)) and an antisense primer with a KpnI site (5′ AAAGGTACCACACTCTCCCCT GTTGAAGC (SEQ ID NO:13)). This PCR product contains the human C kappa coding region without a stop codon. This PCR product is then digested with XbaI and KpnI and inserted in frame into the XbaI and KpnI sites of pcDNA3.1/neo/IgVL, creating pcDNA3.1/neo/IgVL/Kappa(stop-).

The extracellular region of the HLA-DR4 A chain is PCR amplified using a sense primer with anNheI site (5′ AAAGCTAGCATCAAAGAAGAACATGT GATC (SEQ ID NO:14)) and an antisense primer with a HindIII site and a stop codon (5′ TTTAAGCTTTTAGTTCTCTGTAGTCTCTGGGAGAGG (SEQ ID NO: 15)). Following digestion of the PCR product with NheI, an adapter (DRA Adapter 1) is ligated onto the molecule. This adapter is generated by annealing the two single stranded oligos “DRA Adapter 1 sense” (5′CGGC GGCGGCGGCTCTGGCGGCGGCGGCTCTG (SEQ ID NO:16)) and “DRA Adapter 1 Antisense” (5′CTAGCAGAGCCGCCGCCGCCAGAGCCGCCGC CGCCGGTAC (SEQ ID NO:17)). When annealed, this adapter sequence encodes 5′ KpnI overhang (Gly₄Ser)₂ (SEQ ID NO:7) and NheI overhang. After the adapter ligation, the DRA Adapter1/DRA molecule is digested with HindIII and ligated into the KpnI and HindIII sites of pcDNA3.1/neo/IgVL/Kappa/Stop(−), generating pcDNA3.1/neo/IgVL/Kappa/HLA DR. The insertion of Adapter/DRA into the KpnI site is in frame with the kappa coding region. A similar strategy can be employed in order to construct H-HLA-DR4 fusion proteins.

The proper conformation of MHC class II requires that the α and β chains interact to form the peptide binding site. Insertion of the β chain onto the C terminus following the hinge domain, and the a chain onto the C terminus of the light chain would result in the α and β chains being staggered quite far apart. This could result in a misfolding of the molecule and a failure to properly form the peptide binding site. To circumvent this spatial problem a spacer must be constructed that is the approximate length of the H domain. The human IgG3H domain contains approximately 60 amino acids. A spacer containing (Gly₄Ser)₁₂ (SEQ ID NO: 18) provides the proper spacing. This spacer can be generated by synthesis of 2 spacers encoding (Gly₄Ser)₆ (SEQ ID NO: 19). These two spacers can be ligated together and then ligated onto the HLA DR4 A cDNA. The adapter modified DR4 A cDNA is then inserted in frame into the kappa gene as described above (FIGS. 15,16). An alternative to use of this rather long spacer is to employ a shorter hinge region of another IgG heavy chain isotype. This could reduce the length of the required spacer to a more manageable 50 bp.

### Example 4

### Construction of Antibody-Single Chain Class II Fusion Proteins

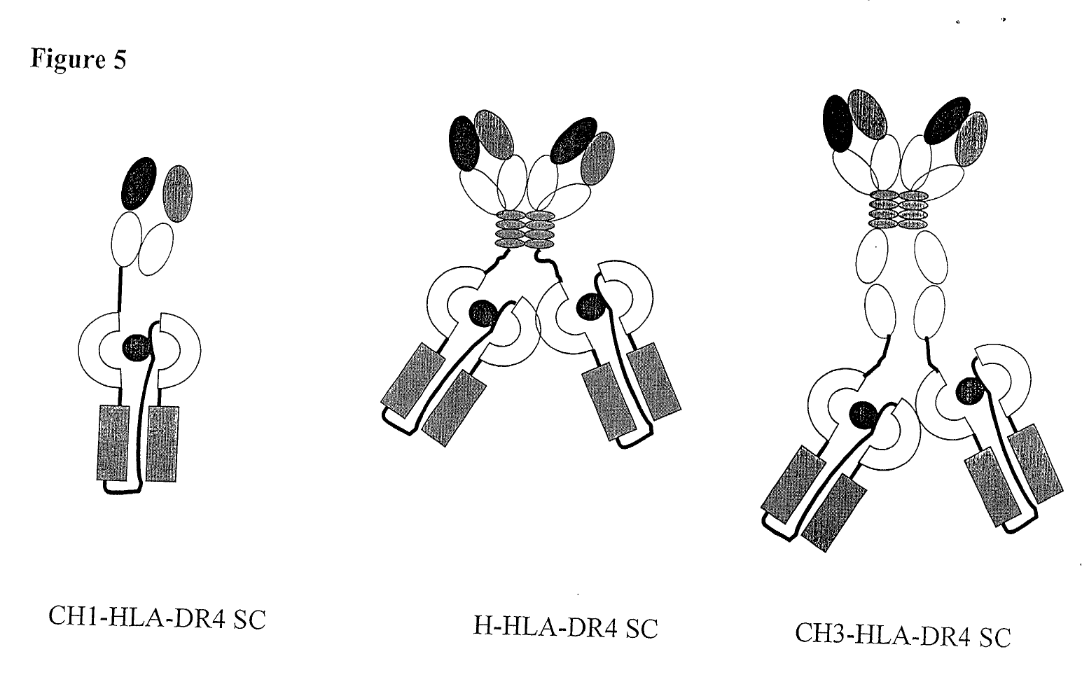

An alternative strategy for construction of Class II-Ig Fusion proteins is to construct single chain class II molecules (Zhu, X. et al., Eur. J. Immunol 27: 1933-1941 (1997)) (FIG. 5). A strategy for construction of Antibody-HLA DR4 single chain fusion protein is described. Modifications of this strategy required for construction of other IgG3-HLA fusion proteins will be evident to those skilled in the art.

The extracellular region of HLA DR4 B chain is PCR amplified with a sense primer containing an NheI site (5′ AAAGCTAGCGGGGACACCC GACCA (SEQ ID NO:10)) and an antisense primer containing a KpnI site without a stop codon (5′ AAAGGTACCCATCTTGCTCTGTGCAGATT(SEQ ID NO:20)). Following PCR amplification, the PCR product is digested with NheI, and the HLA Adapter is ligated onto it. The adapter modified HLA-DR4 B is digested with KpnI and cloned into the EcoRV and KpnI sites of pT7Blue (Novagen), generating pT7Blue.DR4 B. This blunt end ligation leaves the PvuII site at the 5′ end of the molecule intact. The extracellular region of HLA-DR alpha chain is PCR amplified using a sense primer with an SpeI site (5′ AAAACTAGTATCAAAGAAGAACATGTGATC (SEQ ID NO:21)) and an antisense primer with an EcoRI site and a stop codon (5′ TTTGAATTCTTAGTTCTCTGTAGTCTCTGGGAGAGG (SEQ ID NO:22)). Following PCR amplification, the HLA-DRA PCR product is digested with SpeI and ligated to adapter “DRA Adapter 2”. This adapter is formed by annealing the oligos “DRA 2 sense” (5′CGGCGGCGGCGGCTCTGGCGGCGGCGGCA (SEQ ID NO:23)) and “DRA 2 antisense” (5′CTAGTGCCGCCGCCGCCA GAGCCGCCGCCGCCGGTAC (SEQ ID NO:24)). This adapter contains the coding sequence for 5′ KpnI overhang Gly₄SerGly₄ (SEQ ID NO:25) and SpeI overhang.

Following the adapter ligation, the DRA molecule is digested with EcoRI and ligated into the KpnI and EcoRI sites of pT7Blue.DR4 B, generating pT7Blue.HLA DR4 Single Chain. The DR4 single chain DNA is excised from this plasmid by digestion with PvuII and EcoRI and inserted into the SspI and EcoRI sites of pAT3462, generating IgG3-DR4 SC, or inserted into the PvuII and EcoRI sites of pAT4401, generating H-DR4 SC, or inserted into the SnaBI and EcoRI sites of pAT3452, generating CH1-DR4 SC. These Ig/HLA constructs is excised with SalI and BamHI and inserted into the XhoI and BamHI sites of pcDNA3.1/Hygro/IgVH.

### Example 5

### Construction of Antibody-Two Domain MHC Class II Fusion Proteins

Interaction of the β1 and α1 domains of MHC class II forms the peptide binding site for the peptide:MHC complex recognized by a specific TCR. It has been shown that a fusion protein containing only the β1 and α1 domains of MHC class II is able to bind peptide and interact with the TCR (Burrows G. G. et al., J. Immunology 161: 5987-5996 (1998)). A strategy for construction of an antibody—two domain MHC class II fusion protein is described. Modifications of this strategy required for construction of other antibody—two domain MHC class II fusion proteins will be evident to those skilled in the art.

The β1 domain of HLA DR4 B (amino acids 30-124) is PCR amplified using sense primer “DRβ1 sense” (5′ GGGGACACCCGACCA (SEQ ID NO:26)) and anitsense primer “DRβ1 antisense” (5′ GACTCGCCGCTGCACTGT (SEQ ID NO:27)). The α1 Domain of HLA DRA (amino acids 26-109) is PCR amplified using sense primer “DRα1 sense” (5′ ATCAAAGAAG AACATGTGATC (SEQ ID NO:28)) and antisense primer “DRα1 antisense” (5′ GGTGATCGGAGTATAGTTGG (SEQ ID NO:29)). Following the initial PCR amplification, the alpha PCR product is PCR amplified with sense primer “Two Domain DR4 B1-A1 Ligation oligo” (5′ GTGCAGCGGCGAGTCATCAAAG AAGAACATGTGATC (SEQ ID NO:30)) and antisense primer “DRα1 antisense” (5′ GACTCGCCGCTGCACTGT (SEQ ID NO:27)). The “Two Domain DR4 B1-A1 ligation oligo” contains the original DRA sense primer with a 15 bp extension that is complementary to the DRβ1 antisense primer. This alpha PCR product is mixed with the β1 PCR product, denatured, annealed and then extended. The Two Domain Fusion product is then PCR amplified using “DRβ1 sense” primer containing an NheI site (5′ AAAGCTAGCGGGGA CACCCGACCA (SEQ ID NO:31)) and “DRα1 antisense” primer containing an EcoRI site (5′ AAAGAATTCTTAGGTGATCGGAGTATAGTTGG (SEQ ID NO:32). This PCR product is digested with NheI and ligated to the HLA Adapter. The adapter modified Two Domain molecule is digested with PvuII and EcoRI and inserted in frame with the CH1, H or CH3 exons downstream of the selected variable region genes as described herein.

### Example 6

### Construction of Monovalent Antibody—MHC Dimer

For targeting to T or B lymphocytes, it may be advantageous to employ a monovalent rather than a cross-linking antibody specificity that might trigger a broad non-specific inflammatory response. Such monovalent reagents are depicted as CH1 fusion proteins in FIGS. 1-6. The molecules depicted are also monomeric for peptide:MHC complex. Because of the requirement for receptor cross-linking for T cell activation, it would be advantageous to construct molecules containing a single antigen binding site in association with two peptide:MHC complexes. Construction of a monovalent antibody-HLA-A2 dimer is described. Modifications of this strategy required to construct monovalent antibody-MHC dimers with other MHC class I molecules, or with single chain MHC class II molecules, or two domain MHC class II molecules will be evident to those skilled in the art.

These monovalent antibody-MHC dimer molecules are constructed by fusion of the cDNA for an MHC molecule onto the C terminus of the CH1 exon of the antibody heavy chain gene, together with fusion of the cDNA for a second identical MHC molecule onto the C terminus of the light chain gene. For fusion onto the light chain the extracellular region of HLA-A2 is PCR amplified as described above, except that the antisense primer contains a HindIII site instead of an EcoRI site. Following PCR amplification, the HLA-A2 molecule is ligated to the “DRA Adapter 1” as described above. Following this ligation, the adapter modified HLA-A2 molecule is ligated into the KpnI and Hind III sites of pcDNA3.1/neo/IgVL/Kappa(stop-).

### Example 7

### Assay for the in vitro activity of compounds of the invention targeted to dendritic cells

Dendritic cells are the most potent stimulators of T cell responses identified to date. To test in vitro activity of compounds of the invention specifically targeted to dendritic cells, DC are incubated with the relevant compounds and assayed for the ability to activate human autologous T lymphocytes. Immature dendritic cells are prepared from healthy donors according to the method of Bhardwaj and colleagues (Reddy, A. et al.,. Blood 90:3640-3646(1997)). Briefly, PBMC are incubated with neuramimidase-treated sheep erythrocytes and separated into rosetted T cell (ER+) and non-T cell (ER−) fractions. The ER+ fraction is cryopreserved for later use. The ER− fraction (2×10⁶ cells per well) is cultured in serum-free RPMI medium containing 1000U/ml rhGM-CSF, 1000 U/ml rhIL-4 and 1% autologous plasma. This medium is replenished every other day. At day 7, the non-adherent immature DC are harvested from the culture and re-plated in maturation conditions (1000 U/ml GM-CSF, 1000 U/ml IL-4, 1% autologous plasma and 12.5-50% monocyte-conditioned medium) for 2-4 days. Cells manipulated in this manner have morphological and surface characteristics (CD83⁺) of mature DC.

Mature (or immature) DC are pulsed with compounds of the invention, or with free peptide or free MHC/peptide tetramers as controls for a short period followed by cocultivation with autologous T cells in 24 well plates for a period of 7-14 days. In some experiments, these may be total T lymphocytes, but it may also be desirable to fractionate CD4 and CD8 cells using magnetic separation systems (Miltenyi Biotech). Total T lymphocytes are incubated with the appropriate antibody-magnetic bead conjugates to isolate total CD4, CD8, naïve CD4+CD45RA+, naive CD8+CD45RA+, memory CD4+CD45RO+ or memory CD8+CD45RO+ lymphocytes. For naive CD4 and CD8 lymphocytes, a cytokine cocktail consisting of IL-2 (20 U/ml), IL-12 (20 U/ml), IL-18 (10 ng/ml), IFN-gamma (1 ng/ml) and a monoclonal antibody specific for IL-4 (50 ug/ml) is especially potent in enhancing DC activation of cytotoxic T cells in vitro. Following the activation period, CTL activity is assessed in a 4 hour ⁵¹Cr release assay. Other in vitro assays of T cell activation include proliferation (measured by increases in ³H-Thymidine incorporation or colorimetric MTT assay), cytokine secretion (IFN-γ, TNF-α, GM-CSF, IL-2) measured by ELISA, ELISpot, or flow cytometric detection (Luminex bead system). Many of these methods are described in Current Protocols in Immunology (John Wiley & Sons, New York). These and other methods are well known to those practiced in the art. Enhancement of T cell responses to targeted compounds of the invention is determined by comparison to the response to equimolar concentrations of free peptide or untargeted peptide:MHC tetramers.

### Example 8

### Assay for T Cell Proliferation

T cell proliferation can be determined in vitro in a standard assay of ³H-Thymidine uptake and cytotoxic activity can be assayed by ⁵¹Cr release from labeled targets. For example, T cells are treated in vitro with monovalent antibody specific for CD28 costimulator molecules linked to monomeric or polymeric complexes of the influenza matrix peptide (58-66) bound to HLA-A2. Following in vitro culture for 6 days, influenza specific cytotoxic activity is assessed in a standard 4 hour ⁵¹Cr release assay with ⁵¹Cr labeled targets that have been pulsed with either heat killed influenza virus or the specific influenza matrix peptide employed in the stimulating peptide:MHC complexes. The simultaneous delivery to a specific T cell of both ligand for the specific T cell receptor and costimulatory signal via the linked anti-CD28 antibody is expected to greatly enhance that T cell response. Enhancement of T cell responses to compounds of the invention is determined by comparison to the response to equimolar concentrations of the same free peptide or untargeted peptide:MHC complexes.

### Example 9

### Assay for in vivo T Cell Expansion Following Stimulation with Compounds of the Invention

The effect of targeted vaccine complexes on expansion of specific T cells in vivo in either humans or HLA transgenic mice is determined by recovering T cells before and at intervals following immunization with a specific vaccine complex and determining the frequency of T cells specific for the vaccine complex by staining with tetrameric complexes of the same peptide:MHC. Tetramers comprising the same peptide MHC complex of interest are employed in a cell surface immunofluorescence assay as follows. HLA-transgenic mouse spleen, lymph node or peripheral blood cells (collected by tail or retro-orbital bleeding) or human PBMC (1-10⁵ cells per sample) are incubated on ice in the presence of azide with control or experimental tetramers for about 30 minutes. Afterwashing 2-3 times with staining buffer (such as PBS 1% BSA, 0.1% azide) a secondary streptavidin-fluorochrome (FITC, PE, or other fluorochrome) conjugate is added. After incubating for about 30 minutes, the samples are again washed 2-3 times and immunofluorescence is detected using a flow cytometer. These data are compared to pre-vaccination flow cytometric profiles to determine percentage increase in T cell precursor frequency and are repeated multiple times during the course of an experiment or clinical trial.

### Example 10

### In vitro Assays for Tumoricidal Activity of T Cells Specifically Targeted to Tumors by Compounds of the Invention

To demonstrate the ability to redirect cytotoxic T cells to the desired tumor target, tumor cells are incubated with compounds of the invention comprised of a tumor-specific antibody linked to peptide:MHC complexes for which T cells are prevalent (eg HLA-A*0201 associated with influenza matrix peptide 58-66). ⁵¹Cr (100 μCi) is added during this 1 hour incubation to label the tumor cells. Following 2-3 washes, influenza specific CTL restricted to the appropriate MHC molecule (in this case, HLA-A2) are added at various effector to target (E:T) ratios in a 4 hr chromium release assay. Increased tumor lysis in the experimental sample containing compounds of the invention relative to control compounds with irrelevant peptide:MHC complexes or tumor-specific antibody unlinked to peptide:MHC complexes demonstrates that the compound of interest successfully sensitizes tumors to lysis by CTL specific for influenza virus.

The previous paragraph demonstrates redirection of cytotoxic effector function of influenza peptide-specific CTL to uninfected tumor cells by compounds of the invention that comprise a tumor specific antibody and influenza peptide:MHC complexes. To demonstrate the ability of tumor cells treated with the same compound to induce an influenza peptide-specific T cell response, total T cells or CD8⁺CD45RA⁺ naive T cells (1-2×10⁶ per well) are stimulated in 24 well plates with tumor cells (1×10⁵) pulsed with compounds of the invention linked to MHC tetramers with influenza matrix peptide. Cytokines such as IL-2, IL-12, IL-18, IFN-γ may also be added to enhance activation of naive CTL. Induction of cytotoxic T lymphocytes is assessed in a standard ⁵¹Cr release assay, described below.

This same method of targeting peptide:MHC complexes to the tumor cell surface can be employed to enhance MHC-restricted presentation of known tumor-specific peptides; and, more, generally, to overcome immune evasion by tumor cells through downregulation of MHC molecules on the tumor surface. Compounds of the invention that comprise one or more tumor-specific antibodies linked to peptide:MHC complexes would sensitize even tumor targets that have downregulated endogenous MHC to lysis by CTL specific for that same peptide:MHC complex.

### Example 11

### In vivo Assays for Tumoricidal Activity of T Cells Specifically Targeted to Tumors by Compounds of the Invention

In a murine experimental model, compounds of the invention can be targeted to tumor cells through a naturally occurring or transfected tumor membrane marker. For example, BALB/c tumors such as EMT-6 (mammary carcinoma, Rockwell, SC et al., J. Natl. Cancer Inst. 49:735-749 (1972)), Line 1 (small cell lung carcinoma, Yuhas, J. M. et al., Cancer Res. 34:722-728 (1974)) or BCA (fibrosarcoma, Sahasrabudhe, D. M. et al., J. Immunology 151: 6302-6310 (1993)) may be transfected with a model antigen (e.g. chicken egg ovalbumin, OVA) for which antibodies are commercially available or easily made by the skilled artisan. More preferably, a BALB/c mammary tumor such as EMT-6 or SM1 (Hurwitz, A. A. et al., Proc. Nat. Acad. Sci. USA 95:10067-71 (1998)) is employed that expresses the murine homolog of the human C35 protein previously shown to be differentially expressed on the surface of human mammary tumor cells (see Example below). Antibodies or antibody fragments specific for this model antigen may be linked to peptide:MHC tetramers that are either naturally occurring in that tumor, such as the L3 ribosomal protein peptide 48-56 expressed in association with H-2K^{d} in the BCA tumors (see Example below), or a well-characterized pathogenic peptide known to induce a high frequency of high avidity T cells, such as the peptide:MHC complex comprised of the HIV gp160IIIB peptide RGPGRAFVTI in association with H-2D^{d} (Shirai, M. et al., J. Immunol. 148:1657 (1992)).

BALB/c (H-2d) mice with established mammary tumors and/or distant metastases expressing the targeted molecule (e.g. C35) and that have been immunized with a vaccinia recombinant of HIV gp160IIIB are injected with gp160IIIB peptide complexes of H-2D^{d} linked to an anti-C35 antibody specificity for targeting to tumor cells. The effect on tumor growth of treatment with these compounds of the invention is monitored by caliper measurements every other day.

This analysis can be extended to human tumors implanted in immunodeficient (SCID, Rag-1^{-/-}, or Rag-1^{-/-} common γ chain double knockout) mice. Following establishment of tumors in vivo, mice receive an injection(s) of compounds of the invention specific for human tumor antigens conjugated to MHC tetramers bearing the HLA-A2 restricted influenza peptide (or a control peptide). Influenza specific human CTL are adoptively transferred and tumor regression is monitored.

In clinical trials, a standard influenza vaccination may be added to the protocol to increase influenza specific CTL directed at the tumor by compounds of the invention comprising influenza peptide:MHC complexes.

### Example 12

### Inhibition of EAE Induction in SJL Mice

Experimental allergic encephalomyelitis (EAE) is an autoimmune disease in mice and serves as an animal model for multiple sclerosis. Encephalitogenic regions of two proteins, myelin basic protein (MBP91-103) and proteolipoprotein (PLP139-151), have been defined. In the susceptible SJL mouse strain, EAE can be induced to develop following immunization with the encephalitogenic peptide or adoptive transfer of MBP-reactive T cells. To determine whether treatment with a compound of the invention (such a compound comprising MBP 91-103 or PLP 139-151 as the antigenic peptide) will prevent EAE development after T cell activation, SJL mice can be injected with the compound of interest.

To induce EAE in SJL mice with MBP 91-103, mice are immunized with 400 μg of MBP 91-103 in complete Freund's adjuvant on the dorsum. Ten to 14 days later, regional draining lymph node cells are harvested and cultured in 24-well plates at a concentration of 6×10⁶ cells per well in 1.5 ml of RPMI 1640 medium/10% fetal bovine serum/1% penicillin/streptomycin with the addition of MBP at 50 μg/ml. After a 4-day in vitro stimulation, MBP 91-103-reactive T cell blasts are harvested via Ficoll/Hypaque density gradient, washed twice in PBS, and 1.3×10⁷ cells are injected into each mouse. Mice receiving encephalitogenic MBP 91-10³-reactive T cells then receive either 100 μg of a compound of the invention or normal saline on days 0, 3, and 7 i.v. (total dose 300 μg). Clinical and histological evaluations are performed to determine whether the compound of interest inhibited the development of EAE in these mice.

Alternatively, to induce EAE in SJL mice with PLP peptide 139-151, mice are immunized with PLP peptide 139-151 dissolved in PBS and mixed with complete Freund's adjuvant containing Mycobacterium tuberculosis H37Ra at 4 mg/ml in 1:1 ratio. Mice are injected with 150 μg of peptide adjuvant mixture. On the same day and 48 hours later, all animals are given 400 ng of pertussis toxin. Adoptive transfer of EAE are then performed as described above. Clinical and histological evaluations are performed to determine whether the compound of interest inhibited the development of EAE in these mice.

### Example 13

### Effects of the Compounds of the Invention in an Ovalbumin Specific T Cell Hybridoma System

The murine T cell hybridoma, DO 11.10 (Shimonkevitz et al., J. Exp. Med. 158:303 (1983)) expresses on its surface a TCR specific for a 17 amino acid peptide fragment (aa 323-339) derived from chicken egg ovalbumin (Ova). This peptide binds to the murine MHC class II molecule I-A^{d}. DO 11.10 cells respond by producing IL-2, which can then be assayed as a measure of T cell activation.

The compounds of the invention tested in the present example contain MHC class II molecules I-A^{d} as part of the MHC-peptide complex(es). The antigenic peptides used in the present example include Ova 323-339, one of two single-substitution analogs of the Ova peptide (Ova H331R or Ova A332Y), or a peptide from hen egg lysozyme (HEL 74-86). The Ova 323-339, Ova H331R, HEL 74-86 peptides are known to bind I-A^{d} whereas the Ova A332Y analog will serve as anon-binding control (Buus et al., Science 235:1353-1358 (1987); Sette et al., Nature 328:395-399 (1987)). The HEL 74-86 peptide serves as a non-specific negative control.

Briefly, the APCs are incubated with or without the compound of the invention for 3 hours (or more) and then washed extensively to remove unbound compounds. The APCs are then incubated with the DO 11.10 T cell hybridoma (2×10⁵/well) for 24 hours at 37° C. in an atmosphere of 5% CO₂. Cultures are carried out in complete culture medium (RPMI 1640 supplemented with 10% FBS, penicillin/streptomycin, L-glutamine and 5×10⁻⁵ M 2-mercaptoethanol) in 96 well flat bottom microtiter plates.

After 24 hours, culture supernatant is assayed for the presence of IL-2 using the IL-2 dependent murine T cell line CTLL-2. Serial twofold dilutions of each culture supernatant is prepared in completed medium in flat bottomed microtiter plates and 1×10⁴ CTLL-2 cells is added to each well. After 16 to 20 hours the negative control wells (CTLL-2 cultured with medium alone) and positive control wells (CTLL-2 cells cultured with rIL-2) are examined microscopically and at the point at which negative control cells are 90% dead, while positive control cells are still actively proliferating, MTT (2 mg/ml; 25 μl/well) is added and the plates returned to the incubator for an additional 4 hours. At this time, blue crystals formed by MTT in actively metabolizing cells will be dissolved by addition of 150 μl per well of 0.4N HCl in isopropanol per well. After careful mixing, the O.D. at 562 nm is determined using a ELISA plate reader (Ceres-UV900HI). The concentration of IL-2 in experimental wells can be determined by extrapolation from an IL-2 standard curve and then comparison of IL-2 from cultures containing no recombinant protein molecules can be compared to those containing the molecules to be tested and an index of inhibition or stimulation calculated.

Experiments preferably are conducted with peptide antigen pulse conditions of 100 μg/ml and 10 μg/ml and with APC concentrations of 0.5×10⁵/well and 0.1×10⁵/well. This same assay also can be used to identify peptides that function as TCR antagonist or partial agonists.

### Example 14

### Effects of Compounds of the Invention on Antigen Stimulated T Cell Proliferation

Non-transformed T cells isolated from immunized mice require both a peptide/MHC signal as well as co-stimulatory signals in order to proliferate in culture. In this example, T cells are obtained from BALB/c mice (MHC Class II: I-A^{d}). Mice are sacrificed and inguinal and paraaortic lymph nodes removed and rendered into a single cell suspension. The suspension is depleted of antigen presenting cells by incubation on nylon wool and Sephadex G-10 columns, and the resulting purified T cell populations incubated with Click's medium.

Activated B cells from BALB/c mice are used as antigen presenting cells in the proliferation assay. B cells are prepared by culturing spleen cells with 50 μg/ml of LPS for 48 to 72 hours at which time activated cells will be isolated by density gradient centrifugation on Lymphoprep. Activated B cells are then cultured with the compound of interest for 3 hours, washed extensively, fixed with paraformaldehyde to inhibit proliferation of B cells, and added to purified T cells.

The proliferation assay is carried out in 96 well round bottom microtiter plates at 37° C., 5% CO₂ for 3-5 days. Wells are pulsed with 1 μCi of ³H-thymidine for 18 hours prior to termination of cultures and harvested using a Skatron cell harvester. Incorporation of ³H-thymidine into DNA as a measure of T cell proliferation are determined using an LKB liquid scintillation spectrometer. An increase in T cell proliferation following contact with B cells treated with the compound of the invention as compared to a negative control, indicates the compound of interest can stimulate immune responses in a peptide-specific manner.

Alternatively, IL-2 levels can be measured, as described above, at 24 and 48 hours.

### Example 15

### Assay for Immune Induction or Suppression by MHC Fusion Complex

This example uses an animal model of immunization with ovalbumin peptide 323-339 and manipulation of the response to the peptide. The methodology of this example can be applied to a wide variety of compounds of the invention that contain a peptide which can modulate (i.e., suppress or induce) an immune response in an animal.

BALB/c mice (3 per group) are injected i.v. or i.p. with 100 μl of the compound of interest which contains OVA 323-339 as the antigenic peptide.

Ovalbumin peptide (2 mg/ml in PBS) is mixed with 600 μg CpG oligonucleotide, Carson, D. A. and Raz, E. J. Exp. Med. 186:1621-2 (1997) and incomplete Freund's adjuvant in a 1:1 v/v ratio. Fifty μl are injected s.c. into each side of the base of the tail. Seven days after the last injection, lymph nodes (inguinal, paraaortic, cervical, axillary, brachial) are removed and homogenized to obtain a single cell suspension. Lymph nodes from individual mice within a group are processed separately. T cells are purified from lymph node populations by passage of cell suspensions over G-10 and nylon wool to remove accessory cells.

Antigen presenting cells are prepared from the spleens of naive BALB/c mice by homogenizing spleens to obtain a single cell suspension, lysis of erythrocytes using Gey's solution, treatment with mitomycin C (100 μg/ml in RPMI 1640/1% FBS for 1 hour at 37° C.) to inhibit APC proliferation, and 3 washes to remove residual mitomycin C.

Assays for induction of a T cell response are carried out in 96 well round bottom microtiter plates. Two to 4×10⁵ T cells are mixed with 2-4×10⁵ APC. Each T cell/APC combination is incubated, in triplicate, with and without OVA peptide (range 10-200 ng/well) for 3-5 days. Approximately 18 hr before termination of the culture 0.4 μCi of ³H-thymidine is added to each well. The wells are harvested using a Skatron cell harvester and ³H-thymidine incorporation (a measure of DNA synthesis and, therefore, T cell proliferation) is determined using a LKB liquid scintillation spectrometer.

A positive response is evident if the wells containing peptide incorporate significantly more ³thymidine than those without peptide. Typically mice are considered positive where proliferation (in mean cpm) in response to peptide is more than 3 standard deviations greater than the background proliferation without peptide. For each group, mean peptide specific proliferation is calculated by averaging values for each of the 3 mice. Suppression of immunization will typically be considered as having occurred when the experimental group mean is greater than about 3 standard deviations less than the positive control group mean.

### Example 16

### Differential Expression of C35 in Human Breast Carcinoma

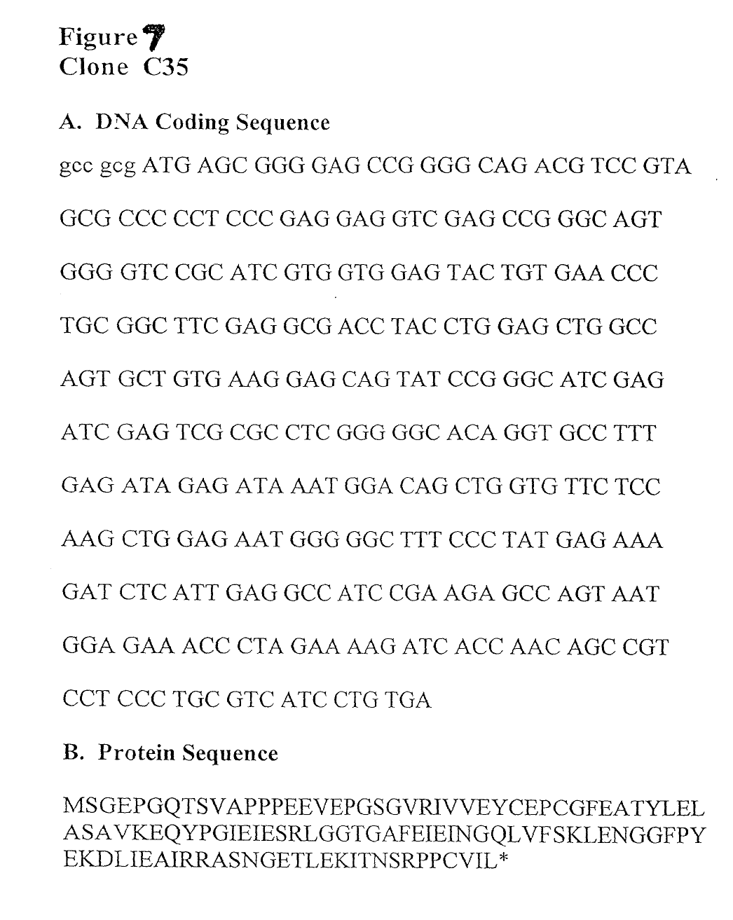

A full-length cDNA representing a gene, C35 (FIG. 7), that is differentially expressed in human breast cancer has been characterized. A 348 base pair DNA fragment of C35 was initially isolated by subtractive hybridization of poly-A RNA from tumor and normal mammary epithelial cell lines derived from the same patient with primary infiltrating intraductal mammary carcinoma. (Band, V. et al., Cancer Res. 50:7351-7357 (1990). Employing primers based on this sequence and that of an overlapping EST sequence (Accession No. W57569), a cDNA that includes the full-length C35 coding sequence was then amplified and cloned from the SKBR2 breast tumor cell line (ATCC, HTB-30). This C35 cDNA includes, in addition to the 348 bp coding sequence, 167 bp of 3′ untranslated region.

Differential expression of the C35 sequence was demonstrated by comparing expression levels of clone C35 in poly-A RNA from cell lines derived from normal mammary epithelium, from two primary breast tumor nodules, and from two metastatic pleural effusions isolated approximately one year later from the same patient (Band, V. et al., Cancer Res. 50:7351-7357 (1990)). Quantitative analysis indicates that the sequence is expressed at a more than 10 fold higher level in tumor cells than in normal mammary epithelium. Low expression levels in a panel of other normal tissues is demonstrated by Northern hybridization. Even though three times as much poly-A RNA was loaded from normal tissues as from the tumor cell lines, little or no expression of RNA homologous to C35 was detected after a comparable 15 hour exposure. Only after an extended 96 hour exposure was low level expression of some homologous sequences detected in normal spleen and kidney tissues. Analysis of expression of C35 homologous sequences in poly-A RNA from three primary infiltrating ductal breast carcinoma from different patients as well as a sample of normal breast epithelium was done. In comparison to normal breast epithelium, sequences homologous to C35 are overexpressed as much as 45 and 25 fold in two of the three primary breast tumors.

An analysis of an immunoprotective tumor antigen expressed in several independently derived murine tumors and, at much reduced levels, in normal mouse tissues was previously conducted. (See U.S. Patent Application No. 60/192,586, the entire contents of which are hereby incorporated herein by reference). In this case, a factor of 9 difference between expression levels in tumor and normal tissues was associated with induction of an immunoprotective tumor-specific response. As discussed above, the expression level of C35 in some human breast cancers relative to normal tissue exceeds a factor of 9, suggesting that C35 might also be immunoprotective against breast cancer in these individuals.

### Example 17

### C35 Specific CTL are Cytolytic for C35 Positive Breast Tumor Cells

Although a gene product may be overexpressed in tumor cells, as is the case for C35, it is immunologically relevant only if peptides derived from that gene product can be processed and presented in association with MHC molecules of the tumor cells. It is conceivable that for any given gene product either no peptides are produced during the cellular degradation process that satisfy the requirements for binding to the MHC molecules expressed by that tumor, or, even if such peptides are generated, that defects in transport or competition for MHC molecules by other tumor peptides would preclude presentation of any peptides from that specific gene product. Even if relevant tumor peptides are processed and presented in association with human MHC in the tumor cells, it must in all cases be determined whether human T cells reactive to these peptides are well-represented in the repertoire or whether T cells may have been rendered tolerant, perhaps due to expression of the same or a related antigen in some other non-homologous normal tissue. For both these reasons, therefore, it is essential to confirm that MHC-restricted, human tumor antigen-specific T cells can be induced by C35 and that they are indeed crossreactive on human tumor cells. Relevant information on this point can be obtained through in vitro stimulation of human T cell responses with recombinant C35 or C35 peptides presented by autologous antigen presenting cells.

A major technical problem in evaluating T cell responses to recombinant gene products is that a strong immune response against the expression vector can block or obscure the recombinant specific response. This is particularly a problem with primary responses that may require multiple cycles of in vitro stimulation. To minimize vector specific responses, it is possible to alternate stimulation by antigen presenting cells infected with different viral vectors recombinant for the same gene product. Convenient vectors include: retroviruses, adenovirus, and pox viruses.

Human PBMC were purified using Ficoll-Paque and subject to rosetting with neuramimidase-treated sheep erythrocytes to isolate monocytes (erythrocyte rosette negative, ER⁻) and T lymphocytes (ER⁺). Dendritic cells were generated from the ER⁻ fraction by culture for 7 days in rhGM-CSF (1000 U/ml) and rhIL-4 (1000 U/ml) with fresh medium and cytokines being added every other day. At day 7, immature dendritic cells were transduced with retrovirus expressing human C35 in the presence of polybrene (1 ug/ml) for 6 hours. Cells were washed and incubated under maturation conditions for 4 days in the presence of 12.5% monocyte conditioned medium, 1000 U/ml rhGM-CSF and 1000 rhU/ml IL-4 and 1% autologous serum. At this point, the dendritic cells were incubated with autologous T lymphocytes (cryopreserved ER fraction) at a ratio of 1 DC:50 T cells for 14 days. Viable T cells were restimulated with autologous, irradiated EBV-B B cells infected at a multiplicity of infection of 1 overnight (16 hours) with a vaccinia recombinant expressing human C35 in the presence of cytokines IL-2 (20U/ml), IL-12 (20 U/ml) and IL-18 (10 ng/ml). Cells were restimulated two more times with autologous EBV-B cells infected with C35-bearing retrovirus in the presence of IL-2 and IL-7 (10 ng/ml). Cytotoxic activity was measured after a total of 4 stimulations by ⁵¹Cr release assay using 5000 targets/well in a 4 hour assay. The results shown in the table below demonstrate specific cytotoxic activity of C35 stimulated T cells against 21NT breast tumor cells that express relatively elevated levels of C35 but not against MDA-MB-231 tumor cells that express the same low levels of C35 as normal nontransformed epithelial cells.

TABLE 7
C35-specific CTL are Cytolytic for C35 Positive Breast Tumor Cells

| | | E:T | |
|---|---|---|---|
| Target Cells | HLA Haplotype | 20:1 | 10:10 |
| Autologous | (Effectors: A2, A11; B8, B35) | (% specific lysis) | |
| EBV-B | A2, A11; B8, B35 | 2 | 1 |
| MDA-MB-231 | A2; B8 | 3 | 1 |
| C35 low (1x) | | | |
| 21NT | A26, A31; B35, B38 | 22 | 10 |
| C35 high (12x) | | | |
| K562 | | 2 | 0 |

### Example 18

### C35 Expression on the Membrane of Breast Carcinoma Cells

To determine whether the C35 polypeptide product is expressed on the surface of tumor cells, a C35 specific antiserum was prepared. BALB/c mice were immunized with syngeneic Line 1 mouse tumor cells that had been transduced with retrovirus encoding human C35. Mice were bled following a series of two or more immunizations. The immune sera were employed to detect surface expression of C35 protein by flow cytometry on three breast tumor cell lines representing high (21NT), intermediate (SKBR3), and low (MDA-MB-231 levels of expression of the C35 transcript in Northern blots. One ×10⁵ breast tumor cells were stained with 3.5 μl of C35 specific antiserum or control, pre-bleed BALB/c serum. After a 30 minute incubation, cells were washed twice with staining buffer (PAB) and incubated with FITC-goat anti-mouse IgG (1 μg/sample) for 30 minutes. Samples were washed and analyzed on an EPICS Elite flow cytometer. The results demonstrated membrane expression of the C35 antigen recognized by the specific immune serum at high levels on tumor line 21NT, intermediate levels for tumor line SKBR3, and undetectable levels in tumor line MDA-MB-231. The high level of reactivity of antibody to membranes of tumor cells that express elevated levels of C35 transcripts suggests that C35 specific antibodies may serve as effective immunotherapeutic agents for the large number of breast carcinoma that overexpress this gene product.

### Example 19

### A Deregulated Ribosomal Protein L3 Gene Encodes a Shared Murine Tumor Rejection Antigen

An antigen discovery technology was developed that allows for the selection of genes encoding CTL epitopes from a cDNA library constructed in a poxyirus. Using this technology, it was determined that a shared murine tumor antigen is encoded by an alternate allele of the ribosomal protein L3 gene. The immunogenic L3 gene is expressed at significant albeit reduced levels in normal tissues including thymus. Immunization with a vaccinia recombinant of the immunogenic L3 cDNA induces protective immunity against tumor challenge. It is of particular interest that a deregulated allele of a housekeeping gene can serve as an immunoprotective antigen and that thymic expression does not preclude immunogenicity of an upregulated tumor product. These observations emphasize that tolerance to a self-protein is not absolute but must be defined in relation to quantitative levels of expression. The ribosomal protein described may be representative of a class of shared tumor antigens that arise as a result of deregulated expression of a self-protein without compromising immune tolerance to normal tissues. Such antigens would be suitable for immunotherapy of cancer in vital organs.

Total RNA was isolated from BCA 39 tumor cells using the Perfect RNA Total RNA Isolation Kit (5 Prime 3 Prime, Inc., Boulder, Colo.). Poly A+ mRNA was isolated from the total RNA using Dynabeads (Dynal, Lake Success, N.Y.). Two micrograms of poly A+ mRNA was converted to double stranded cDNA using the Great Lengths cDNA Synthesis Kit (Clontech, Palo Alto, Calif.). The double stranded cDNA was then inserted in vaccinia virus vector v7.5/tk (5).

BALB/cByJ (Jackson Labs) mice were immunized intraperitoneally with 2×10⁶ irradiated (6,500 cGy) BCA 34 cells. Two weeks later, the mice were boosted by subcutaneous injection of 2×10⁶ irradiated BCA 34 cells. One week following the second immunization, splenocytes were harvested, divided into 12 parts and cultured in 12 well plates with 6×10⁵ irradiated (10,000 cGy), mitomycin C treated BCA 34 cells per well. At weekly intervals, viable T cells were purified using Lympholyte-M (Accurate Chemical, Westbury, N.Y.) and cultured in 12 well plates at 1.5×10⁶ T cells per well. To each well was also added 4×10⁶ irradiated (5000 cGy) BALB/c spleen, along with 6×10⁵ irradiated, mitomycin C treated BCA 34 cells.

A specific vaccinia recombinant that encodes the well characterized ovalbumin 257-264 peptide (SIINFEKL) (SEQ ID NO:35) that is immunodominant in association with H-2K^{b} was diluted with non-recombinant virus so that it initially constituted either 0.2%, 0.01%, or 0.001% of total viral pfu. An adherent monolayer of MC57G cells (H-2) were infected with this viral mix at m.o.i.=1 (approximately 5×10⁵ cells/well). Following 12 hours infection, ovalbumin peptide-specific CTL, derived by repeated in vitro stimulation of ovalbumin primed splenic T cells with the immunodominant SIINFEKL (SEQ ID NO:35) peptide, were added. During this incubation, those adherent cells which were infected with a recombinant particle that expresses the ovalbumin peptide are targeted by specific cytotoxic T cells and undergo a lytic event which causes them to be released from the monolayer. Following incubation with CTL, the monolayer is gently washed, and both floating cells and the remaining adherent cells are separately harvested. Virus extracted from each cell population was titred for the frequency of recombinant (BRdU resistant) viral pfu. Virus extracted from floating cells was then used as input to another enrichment cycle with fresh adherent MC57G cells and ovalbumin peptide-specific CTL. It was observed that following enrichment of VVova to greater than 10% of total virus, further enrichment of the recombinant virus was accelerated if the m.o.i. in succeeding cycles was reduced from 1 to 0.1.

Confluent monolayers of BCN in wells of a 12 well plate were infected with moi=1.0 vaccinia BCA39 cDNA library. At 12 hours post-infection the monolayers were washed 3×with media, and 2.5×10⁶ CTL were added to the wells in a 250 μl volume. The T cells and targets were incubated at 37° C. for 4 hours. Following the incubation the supernatant was harvested, and the monolayer gently washed 3×with 250 μl media. Virus was released from the cells by freeze/thaw, and titers determined by plaque assay on BSC1 cells. The selected virus population (floating cells in cultures that received specific T cells) was amplified on BSC1 cells in one well of a 12 well plate for 2 days. The virus was then harvested and titered. This viral stock was subjected to three additional enrichment cycles. The selected virus population was not amplified prior to the next cycle.

Virus from the fourth enrichment cycle was divided into 40 pools of 5 pfu each. Each pool was amplified on BSC1 cells in a 96 well plate, with 1 pool/well. After 4 days the virus was harvested (P 1), and used to infect monolayers of BCN in a 96 well plate at moi=5, with 1 pool per well. As a control, a monolayer of BCN was infected with moi=5 vNotI/tk (Merschlinsky et al., Virology 190:522 (1992)). At 5 hours post-infection, 2×10⁴ washed CTL were added to each well. The final volume in each well was 225 μl. The cells were incubated at 37° C. for 18 hours. The cells were then pelleted by centrifugation, 150 μl supernatant was harvested and tested for IFNg by ELISA. Twenty seven of the forty pools of 5 pfu were positive for the ability to stimulate CTL. Suggesting, by Poisson analysis, that specific recombinants were enriched to greater than 20%. Individual clones were picked from 5 positive pools and assayed as above.

Monolayers of B/C.N in a 6 well plate were infected with moi=1.0 of v7.5/tk, vF5.8, or vH2.16. At 14 hours post-infection cells were harvested along with the control targets: B/C.N, BCA 34, and BCA 39. The target cells were labeled with 100 microcuries ⁵¹Cr (Dupont, Boston, Mass.) for 1 hour at 37° C., and 10⁴ cells were added to wells of a 96 well round bottom plate in quadruplicate. Tumor specific CTL were added to target cells at the indicated ratios. Cells were incubated at 37° C. for 4 hours. Supernatants were harvested and ⁵¹Cr release determined. Spontaneous release was derived by incubating target cells with media alone. Maximal release was determined by incubating target cells with 5% Triton ×100. Percentage of specific lysis was calculated using the formula: % specific lysis=((experimental release-spontaneous release)/(maximal release-spontaneous release))×100. In each case the mean of quadruplicate wells was used in the above formula.

Two μg of total RNA was converted to cDNA using a dT primer and Superscript II Reverse Transcriptase (BRL, Gaithersburg, Md.). cDNA was used as the template for a PCR using L3 specific primers; L3.F1.S (CGGCGAGATGT CTCACAGGA (SEQ ID NO:36)) and L3.F1.AS (ACCCCACCATCTGCA CAAAG (SEQ ID NO:37)); and Klentaq DNA Polymerase Mix (Clontech) in a 20 μl final volume. Reaction conditions included an initial denaturation step of 94° C. for 3 minutes, followed by 30 cycles of: 94° C. 30 seconds, 60° C. for 30 seconds, 68° C. for 2 minutes. These PCR products contained the region of L3 between position 3 and 1252. The PCR products were purified using Centricon 100 columns (Amicon, Beverly, Mass.), digested with Sau3AI, and resolved on a 3% Agarose/ethidium bromide gel.

Adult female BALB/cByJ mice (2 mice per group) were immunized by subcutaneous injection of 5×10⁶ pfu of vH2.16, orv7.5/tk. Seven days following the immunization, splenocytes were harvested and cultured in 12 well plates along with 1 μM peptide L3₄₈₋₅₆(154). After seven days, the viable T cells were purified using Lympholyte-M, and 1×10⁶ T cells were added to wells of a 12 well plate along with 1 micromolar peptide and 4×10⁶ irradiated (5000 cGy) BALB/c spleen cells per well.

Adult female BALB/cByJ mice were immunized by subcutaneous injection of 1×10⁶ pfu of vH2.16, vPKIa, v7.5/tk or Phosphate Buffered Saline. Secondary immunizations were given 21 days later. Mice were challenged with tumor by subcutaneous injection of 2×10⁵ BCA 34 cells twenty one (primary immunization only) or fourteen days following immunization.

Prospects for development of broadly effective tumor vaccines have been advanced by evidence that several self-proteins can be recognized as tumor antigens by immune T cells (Van den Eynde et al., J. Exp. Med. 173:1373 (1991); M. B. Bloom et al., J. Exp. Med. 185:453 (1997); Van Der Bruggen et al., Science 254:1643 (1991); Gaugler et al., J. Exp. Med. 179:921 (1994); Boel et al., Immunity 2:167 (1995); Van Den Eynde et al., J. Exp. Med. 182:689 (1995); Kawakami et al., Proc. Natl. Acad. Sci. U.S.A. 91:3515 (1994); Kawakami et al., Proc. Natl. Acad. Sci. U.S.A. 91:6458 (1994); Brichard et al., J. Exp. Med. 178:489(1993)). Such normal, nonmutated gene products may serve as common target antigens in tumors of certain types arising in different individuals. Clinical evidence for induction of protective immunity following vaccination with such shared tumor antigens is, currently, very limited (Marchand et al., Int. J. Cancer 80:219 (1999); Rosenberg et al., Nat. Med. 4:321 (1998); Overwijk et al., Proc. Natl. Acad. Sci. 96:2982 (1999); Brandle et al., Eur. J. Immunol. 28:4010 (1998)). It is, moreover, not at all clear whether the T cell responses to these self-proteins represent a surprising breakdown in immunological tolerance or are a consequence of qualitative or quantitative changes in the expression of the self-proteins in tumor cells. In the latter case, normal tissue tolerance could be maintained and vaccine induced immunity to self-proteins whose expression is systematically altered in tumors might be applicable even to cancer of vital organs.

A ribosomal protein allele that is systematically deregulated in multiple murine tumors during the transformation process was shown to be a tumor rejection antigen and the principal correlate of immunogenicity is a dramatic change in quantitative expression in tumors relative to normal tissues and thymus.

Previously, it was reported that cross-protective immunity is induced among three independently derived murine tumor cell lines (Sahasrabudhe et al., J. Immunology 151:6302 (1993)). These tumors, BCA 22, BCA 34, and BCA 39 were derived by in vitro mutagenesis of independent subcultures of the B/C.N line, a cloned, immortalized, anchorage-dependent, contact inhibited, nontumorigenic fibroblast cell line derived from a BALB/c embryo (Collins et al., Nature 299:169 (1982); Lin et al., JNCI 74:1025 (1985)). Strikingly, immunization with any of these tumor cell lines, but not with B/C.N provided protection against challenge with not only homologous tumor cells, but also against challenge with the heterologous tumor cell lines. Following immunization with any of these three tumor cell lines, CD8+cytolytic T lymphocyte (CTL) lines and clones could be generated which in vitro displayed crossreactive specificity for the same three tumors, but not for the non-tumorigenic B/C.N cells from which they derived.

In order to move from an immunological definition to a molecular definition of this shared tumor antigen(s), a novel and efficient method for the identification of genes that encode CTL target epitopes was developed. In this approach a cDNA library from the BCA 39 tumor cell line was constructed in a modified vaccinia virus expression vector (Merchlinsky et al., Virology 238:444 (1997); E. Smith et al., Manuscript in preparation). Five hundred thousand plaque forming units (pfu) of this library were used to infect a monolayer of antigen-negative B/C.N cells at a multiplicity of infection (moi) of 1. Following 12 hours infection, BCA 34 tumor specific CTL were added to the target cell monolayer at an effector to target ratio that gives approximately 50% lysis in a standard ⁵¹Cr release assay. CTL specific for the heterologous BCA 34 tumor cell line were used in order to facilitate the identification of antigen(s) which are shared between these two tumor cell lines. Since adherence is an energy dependent process, it was expected that cells that undergo a CTL mediated lytic event would come off of the monolayer and could be recovered in the supernatant. By harvesting virus from floating cells following cell mediated lymphocytotoxicity (CML), it was possible to enrich for viral recombinants that had sensitized the host cell to lysis. An essential feature of this procedure is that it lends itself to repetition. The virus harvested following one cycle of enrichment can be used as input for additional cycles of selection using fresh monolayers and fresh CTL until the desired level of enrichment has been achieved. In a model experiment with CTL specific for a known recombinant, it was possible to demonstate that specific recombinants could be enriched from an initial dilution of 0.001% to approximately 20% in 6 cycles of selection (Table 8). At this level it is a simple matter to pick individual plaques for further characterization.

TABLE 8
Multiple Cycles of Enrichment for VVova: A vaccinia
cocktail composed of wild type vNotl/tk (tk+) spiked with the indicated
concentrations of VVova (tk−) was subjected to CML Selection (12)

| | Enrichment | % VVova in Floating Cells | | |
|---|---|---|---|---|
| | Cycle # | Expt. 1 | Expt. 2 | Expt. 3 |
| moi = 1 | 0 | 0.2 | 0.01 | 0.001 |
| | 1 | 2.1 | 0.3 | nd |
| | 2 | 4.7 | 1.1 | nd |
| | 3 | 9.1 | 4.9 | nd |
| | 4 | 14.3 | 17.9 | 1.4 |
| | 5 | 24.6 | | 3.3 |
| | 6 | | | 18.6 |
| moi = 0.1 | 5 | 48.8 | 39.3 | |

% VVova = (Titer with BudR/Titer without BudR) × 100
nd = not determined

The poxyirus expression library was subjected to 4 cycles of selection with tumor-specific CTL. Individual plaques of the selected viral recombinants were expanded and used to infect separate cultures of B/C.N cells. These cells were assayed for ability to stimulate specific CTL to secrete interferon gamma (INFγ), or for sensitization to lysis by the tumor-specific CTL. Ten viral clones were isolated, all of which conferred upon B/C.N the ability to stimulate a line of tumor-specific CTL to secrete IFNγ. All 10 clones contained the same sized (1,300 bp) insert (Smith et al., unpublished data). Sequence analysis confirmed that clones F5.8 and H2.16 contained the same full-length cDNA. It appeared, therefore, that all ten clones were recombinant for the same cDNA. In all, 6 of 6 CTL lines that were generated by immunization with BCA 34 demonstrated specificity for this antigen.

A search of GenBank revealed that this cDNA is highly homologous to the murine ribosomal protein L3 gene (Peckham et al., Genes and Development 3:2062 (1989)). Sequencing the entire H2.16 clone revealed only a single nucleotide substitution that coded for an amino acid change when compared to the published L3 gene sequence. This C170T substitution generates a Threonine to Isoleucine substitution at amino acid position 54. The F5.8 clone also contained this nucleotide substitution.

Since CTL recognize antigen as peptide presented by a MHC molecule, it was of interest to identify the peptide epitope recognized by these MHC class I-restricted tumor-specific CD8+ T cells. It was considered likely that the altered amino acid (Ile 54) would be included in the peptide recognized by the CTL. This hypothesis was supported by the demonstration that a vaccinia virus clone recombinant for only the first 199 bp (63 amino acids) of H2.16 (vH2₁₉₉) was able to sensitize B/C.N to lysis by tumor-specific CTL (Smith et al., unpublished data). A Computer screen of peptide-binding motifs suggested that there are two epitopes encoded within this region that could associate with high affinity to the MHC class I molecule Kd (Parker et al., J. Immunology 152:163 (1994)). These two peptides, L3₄₅₋₅₄ (154) and L3₄₈₋₅₆ (154) were synthesized and tested for the ability to sensitize B/C.N cells to lysis by tumor-specific CTL. Peptide L3₄₈₋₅₆ (I54) sensitized B/C.N to lysis, while L3₄₅₋₅₄ (154), and the wild type L3₄₈₋₅₆ (T54) did not. It was determined that 10 nM L3₄₈₋₅₆ (154) was sufficient to sensitize targets to lysis by CTL, whereas 100 mM L3₄₈₋₅₆ (T54) did not. These results demonstrate that peptide L3₄₈₋₅₆ (154) is a target epitope recognized by the tumor-specific CTL.

To analyze expression of the different L3 gene products, oligo-dT primed cDNA was synthesized from RNA of tumors and the B/C.N cell line from which they derived. The first strand cDNA was subjected to PCR amplification using a pair of primers which amplify nearly the entire mouse L3 mRNA. Sequence analysis of these PCR products showed that B/C.N and BCB13 L3 cDNA contained a C at position 170 (same as published sequence). BCB13 is a tumor cell line that was derived from the B/C.N cell line, but that is not immunologically cross-protective with the BCA tumor cell lines (Sahasrabudhe et al., J. Immunology 151:6302 (1993)). Sequence analysis of the PCR products from the crossreactive BCA 39, BCA 34, and BCA 22 tumors suggested that these cell lines express two different species of L3 mRNA. One species contains a C at 170, and the other contains a T at 170, as in the H2.16 clone. The sequence of all L3 cDNAs were identical except for this one base substitution.

There are two possible ways to account for the origin of the new L3 RNA in tumor cells. Either the L3 (C170T)gene expressed in these tumors is a somatic mutant of the wild type gene or there are multiple germ line alleles of L3, at least one of which gives rise to an immunogenic product when deregulated during the process of tumor transformation. The first hypothesis was considered unlikely because the crossreactive BCA 39, BCA 34, and BCA 22 tumors were independently derived. It would be remarkable if the same mutant epitope was generated in all three tumors. On the other hand, Southern blots of different restriction digests of genomic DNA from BCA 39 and B/C.N suggested that there are multiple copies of the L3 gene in the mouse genome (Smith et al., unpublished data). The L3 gene has also been reported to be multi-allelic in both the rat and the cow (Kuwano et al., Biochemical and Biophysical Research Communications 187:58 (1992); Simonic et al., Biochemica et Biophysica Acta 1219:706 (1994)). Further analysis was required to test the hypothesis that different L3 alleles in the germ line are subject to differential regulation in tumors and normal cells.

The nucleotide sequence of the published L3 from position 168 to 171 is GACC. The sequence of H2.16 in this same region is GATC. This new palindrome is the recognition sequence for a number of restriction endonucleases, including Sau3AI. A Sau3A I digest of L3 is expected to generate fragments of 200, 355, 348, 289, and 84 base pairs, while a Sau 3A I digest of H2.16 would generate a 168 bp fragment in place of the 200 bp fragment. This difference in the Sau3AI digestion products was used to confirm that the three BCA cell lines express at least two different L3 alleles. The L3 RT-PCR products from all 5 cell lines and thymus RNA were digested with Sau 3AI and analyzed on an agarose gel. All 3 BCA lines express both versions of L3. Remarkably, when this assay was repeated using greater amounts of starting material, the 168 bp fragment was also detectable in the digests of B/C.N, BCB13 and normal thymus cDNA (Smith et al., unpublished data). To enhance the sensitivity of this assay, the PCR was repeated using a P³² end-labeled 5′ L3 specific primer. The radiolabeled PCR products were digested with Sau3AI and resolved on an agarose gel. B/C.N, BCB13 and thymus contain the 168 bp fragment. Quantitative analysis indicates that the ratio of 200 bp: 168 bp fragments in the BCA tumors is 2:1 while the ratio of the same fragments detected in B/C.N, BCB13, and thymus is approximately 20:1. Low levels of expression of this immunogenic L3 allele was also observed when RNA from kidney, heart, and skeletal muscle was analyzed (Smith et al., unpublished data). These results suggest that gene deregulation associated with the transformation process in the crossreactive tumors leads to the expression of higher levels of this germ line L3 (C170T) allele, and that this altered L3 gene was not generated by somatic mutation of the L3 gene that is predominantly expressed in normal tissues. This new L3 allele (C170T), has been termed the immunogenic L3 allele (iL3).

It is particularly intriguing that the immunogenic L3 allele is also expressed, albeit at a 10 fold reduced level, in normal thymus. This level of expression is evidently not sufficient to tolerize all T cells with functional avidity for the level of deregulated iL3 expressed in some tumors. The observation that although B/C.N and BCB13 express low levels of iL3, they are not susceptible to lysis by the tumor specific CTL suggests, however, that higher affinity T cells have been tolerized. This appears to be the first instance in which a tumor antigen has been reported to be expressed in the thymus. These observations emphasize that tolerance to a self-protein is not absolute but must be defined in relation to quantitative levels of expression (Targoni et al., J. Exp. Med. 187:2055 (1998); C. J. Harrington et al., Immunity 8:571 (1998)).

If broadly effective vaccines are to be developed based on expression of shared tumor antigens, then it is critical to demonstrate that such antigens can be immunoprotective. The largest number of shared antigens have been identified for human tumors, but clinical immunotherapy trials employing these antigens have so far been inconclusive, in part because of uncertainty regarding optimal vaccination strategies (Pardoll, D. M., Nat. Med. 4:525 (1998)). In mice, where immunotherapeutic strategies could be more thoroughly investigated, very few shared tumor antigens have been identified. It was, therefore, of considerable interest to determine whether immunization with iL3 recombinant vaccinia virus would induce tumor specific CTL and protect mice from tumor challenge (Overwijk et al., Proc. Natl. Acad. Sci. 96:2982 (1999); Moss, B., Science 252:1662 (1991); Irvine et al., J. Immunology 154:4651 (1995); McCabe et al., Cancer Research 55:1741 (1995); Estin et al., Proc. Natl. Acad. Sci. 85:1052 (1988); J. Kantor et al., JNCI 84:1084 (1992); V. Bronte et al., Proc. Natl. Acad. Sci. 94:3183 (1997)). Immunization of BALB/c mice with vaccinia virus recombinant for the iL3 gene (H2.16) generated CTL that were able to lyse both BCA 34 and BCA 39 tumor cells, but not B/C.N in vitro. Mice immunized twice or even once with vaccinia virus recombinant for iL3 were able to reject challenge with BCA 34 tumor cells. Mice immunized with empty viral vector, or control vaccinia recombinant for the Inhibitor Protein of cAMP-dependent Protein Kinase (PKIa) were unable to reject this tumor challenge (Olsen, S. R. and Uhler, M. D., J. Biol. Chem. 266:11158 (1991); Mueller et al., Manuscript in Preparation). These results demonstrate that the iL3 self-protein is an immunoprotective tumor antigen.

A new strategy was developed to identify genes that encode CTL epitopes based on CTL-mediated selection from a tumor cDNA library in a modified vaccinia virus vector (Merchlinsky et al., Virology 238:444 (1997); E. Smith et al., manuscript in preparation). This strategy was applied to identify a deregulated housekeeping gene that encodes a tumor rejection antigen shared by three independently derived murine tumors. This ribosomal protein may be representative of a larger class of immunoprotective shared tumor antigens that become immunogenic as a result of deregulated expression of self-proteins without compromising immune tolerance to normal tissues. Such antigens would be well suited for immunotherapy of cancer in vital organs.

### Example 20

### T Cell Stimulation in Mice Treated with Compounds of the Invention

The effects of compounds of the invention on clonal expansion of peptide-specific T cell lines in vivo can be suitably examined in accordance with the following assay.

5 BALB/c mice are injected intraperitoneally with 10-100 μg of a compound of interest in PBS and 24 hours later injected subcutaneously at the base of the tail with 50 μg of peptide-KLH conjugate. The peptide in the antigenic peptide-KLH conjugate is the same antigenic peptide in the compound of interest. 5 BALB/c mice are injected with peptide-KLH conjugate alone. 5 BALB/c mice re injected with PBS. These injections are repeated 6 and 7 days later. Seven days after completion of the second set of injections, the mice are sacrificed. The inguinal and paraaortic lymph nodes are removed and rendered into a single cell suspension.

The suspension is depleted of antigen presenting cells by incubation on nylon wool and Sephadex G-10 columns, and the resulting purified T cell populations incubated with APCs pulsed with the peptide. Activated B cells from BALB/c mice are used at antigen presenting cells in the proliferation assay. B cells are prepared by culturing spleen cells with 50 μg/ml of LPS for 48 to 72 hours at which time activated cells are isolated by density gradient centrifugation on Lymphoprep. Activated B cells are then pulsed with the peptide for 3 hours, washed extensively, fixed with paraformaldehyde to inhibit proliferation of B cells, and added to purified T cells from each panel of mice.

The proliferation assay is carried out in 96 well round bottom microtiter plates at 37° C., 5% CO₂ for 3-5 days. Wells are pulsed with 1 μCi of ³H-thymidine for 18 hours prior to termination of cultures and harvested using a Skatron cell harvester. Incorporation of ³H-thymidine into DNA as a measure of T cell proliferation is determined using an LKB liquid scintillation spectrometer. The degree of peptide-reactive T cell proliferation is indicative of the T cell responses (i.e. of clonal expansion) that took place in the mice following immunization.

### Example 21

### Detection of Peptide Specific T Cells Following Induction of Immune Response

In order to determine whether injection of a compound of the invention has successfully immunized mice to mount a T cell response to ovalbumin, an ovalbumin specific T cell proliferation assay can be employed. Mice are immunized by the protocol described in Example 20 and T cells are prepared from the inguinal and paraaortic lymph nodes 6 days after the second immunization.

The suspension is depleted of antigen presenting cells by incubation on nylon wool and Sephadex G-10 columns, and the resulting purified T cell populations incubated with APCs pulsed with the antigenic peptide. Activated B cells from BALB/c mice are used as antigen presenting cells in the proliferation assay. B cells are prepared by culturing spleen cells with 50 μg/ml of LPS for 48 to 72 hours at which time activated cells are isolated by density gradient centrifugation on Lymphoprep. Activated B cells are then pulsed with the antigenic peptide for 3 hours, washed extensively, fixed with paraformaldehyde to inhibit proliferation of B cells, and added to purified T cells.

The proliferation assay is carried out in 96 well round bottom microtiter plates at 37° C., 5% CO₂ for 3-5 days. Wells are pulsed with 1 μCi of ³H-thymidine for 18 hours prior to termination of cultures and harvested using a Skatorn cell harvester. Incorporation of ³H-thymidine into DNA as a measure of T cell proliferation is determined using an LKB liquid scintillation spectrometer. The degree of peptide-reactive T cell proliferation is indicative of the T cell responses (i.e. of clonal expansion) that took place in the mice following immunization.

### Example 22

### Antibody Dependent Targeting of Exogenous MHC:peptide Complexes to Cell Surface Membranes is Sufficient to Stimulate Specific T Lymphocytes

Biotinylated anti-CD19 antibody (1 μl of 0.7 μg/ml) is added to 5×10⁵ EBV-B cells in a total volume of 0.1 ml. CD19 is a well characterized surface membrane marker of EBV-B cells. After 30 min incubation on ice, cells are washed twice with 1 ml cold PBS+5% BSA. Streptavidin (1 μl of 0.07 μg/ml) is added for another 30 min incubation followed by two more washes. Finally, a biotinylated monomer of H-2D^{d} bound to an immunodominant HIV peptide (p18) is added for a 30 min incubation. The complex of biotinylated-anti-CD19: streptavidin: H-2D^{d}/p18 is assembled step-wise in a 4:1:4 molar ratio. Samples are washed and resuspended in a final volume of 100 μl RPMI-1640 complete medium and transferred to a 96 well plate. Either T cells specific for the immunodominant gp160 epitope, p18, in association with H-2D^{d} or control T cells specific for an unrelated peptide in association with H-2K^{d} (BCA39) are added at 10⁵ cells/well in 100 μl complete medium. Induction of IFNγ secretion by T cells is determined by IFNγ-specific ELISA assay following an overnight incubation. The data show the mean and standard deviation of relative IFNγ secretion as OD 450-OD 570 employing a standard ELISA assay protocol. Each measurement is a replicate of 4 wells. Background secretion in the absence of the assembled MHC:peptide complex is subtracted. The difference in the induction of IFNγ secretion by specific and control T cells is significant with p<0.01 by Student's single tail T test. gp160-specific T cells had a relative IFNγ secretion of 0.94 (±0.26). BCA39-specific T cells had a relative IFNγ secretion of 0.29 (±0.19).

It will be clear that the invention may be practiced otherwise than as particularly described in the foregoing description and examples.

Numerous modifications and variations of the present invention are possible in light of the above teachings and, therefore, are within the scope of the appended claims.

The entire disclosure of all publications (including patents, patent applications, journal articles, laboratory manuals, books, or other documents) cited herein are hereby incorporated by reference.

What is claimed is:

1. A compound comprising:
(a) one or more MHC-peptide complexes; and
(b) an antibody or a fragment thereof specific for a cell surface marker;
wherein said MHC-peptide complexes comprise an MHC class I α chain or fragment thereof, a β₂-microglobulin molecule or fragment thereof, and an antigenic peptide bound in the MHC groove; and
wherein said MHC-peptide complexes are linked to the carboxyl terminus of said antibody or fragment thereof.

2. The compound of claim 1, wherein said cell surface marker is a cell surface marker of a professional antigen presenting cell.

3. The compound of claim 2, wherein said professional antigen presenting cell is a dendritic cell.

4. The compound of claim 3, wherein said cell surface marker is selected from the group consisting of CD83, CMRF-44, CMRF-56 and DEC-205.

5. The compound of claim 1, wherein said cell surface marker is a cell surface marker of a tumor cell.

6. The compound of claim 1, wherein said cell surface marker is a cell surface marker of an epithelial cell.

7. The compound of claim 1, wherein said cell surface marker is a cell surface marker of a fibroblast.

8. The compound of claim 1, wherein said cell surface marker is a cell surface marker of a T cell.

9. The compound of claim 8, wherein said cell surface marker is selected from the group consisting of CD28, CTLA-4 and CD25.

10. The compound of claim 1, wherein said antigenic peptide is derived from a cancer cell.

11. The compound of claim 1, wherein said antigenic peptide is derived from an infectious agent or from infected cells.

12. The compound of claim 1, wherein said antigenic peptide is derived from the target tissue of an autoimmune disease.

13. The compound of claim 5, wherein said antigenic peptide is derived from a cancer cell.

14. A compound comprising:
(a) one or more MHC-peptide complexes; and
(b) an antibody or a fragment thereof specific for a cell surface marker;
wherein said MHC-peptide complexes comprise an MHC class I α chain or fragment thereof, a β₂-microglobulin molecule or fragment thereof, and an antigenic peptide bound in the MHC groove; and
wherein said MHC class I α chain or fragment thereof of said MHC-peptide complexes are linked to the carboxyl terminus of said antibody or fragment thereof.

15. The compound of claim 14, wherein said cell surface marker is a cell surface marker of a professional antigen presenting cell.

16. The compound of claim 15, wherein said professional antigen presenting cell is a dendritic cell.

17. The compound of claim 16, wherein said cell surface marker is selected from the group consisting of CD83, CMRF-44, CMRF-56 and DEC-205.

18. The compound of claim 14, wherein said cell surface marker is a cell surface marker of a tumor cell.

19. The compound of claim 14, wherein said cell surface marker is a cell surface marker of an epithelial cell.

20. The compound of claim 14, wherein said cell surface marker is a cell surface marker of a fibroblast.

21. The compound of claim 14, wherein said cell surface marker is a cell surface marker of a T cell l.

22. The compound of claim 21, wherein said cell surface marker is selected from the group consisting of CD28, CTLA-4 and CD25.

23. The compound of claim 14, wherein said antigenic peptide is derived from a cancer cell.

24. The compound of claim 14, wherein said antigenic peptide is derived from an infectious agent or from infected cells.

25. The compound of claim 14, wherein said antigenic peptide is derived from the target tissue of an autoimmune disease.

26. The compound of claim 18, wherein said antigenic peptide is derived from a cancer cell.

27. A compound comprising:
(a) one or more MHC-peptide complexes; and
(b) an antibody or fragment thereof specific for a cell surface marker;
wherein said MHC-peptide complexes comprise an MHC class II α chain or fragment thereof, an MHC class II chain or fragment thereof, and an antigenic peptide bound in the MHC groove; and
wherein at least one chain or fragment thereof of said MHC-peptide complexes are linked to the carboxyl terminus of said antibody or fragment thereof.

28. The compound of claim 27, wherein said cell surface marker is a cell surface marker of a professional antigen presenting cell.

29. The compound of claim 28, wherein said professional antigen presenting cell is a dendritic cell.

30. The compound of claim 29, wherein said cell surface marker is selected from the group consisting of CD83, CMRF-44, CMRF-56 and DEC-205.

31. The compound of claim 27, wherein said cell surface marker is a cell surface marker of a tumor cell.

32. The compound of claim 27, wherein said cell surface marker is a cell surface marker of an epithelial cell.

33. The compound of claim 27, wherein said cell surface marker is a cell surface marker of a fibroblast.

34. The compound of claim 27, wherein said cell surface marker is a cell surface marker of a T cell.

35. The compound of claim 34, wherein said cell surface marker is selected from the group consisting of CD28, CTLA-4 and CD25.

36. The compound of claim 27, wherein said antigenic peptide is derived from a cancer cell.

37. The compound of claim 27, wherein said antigenic peptide is derived from an infectious agent or from infected cells.

38. The compound of claim 27, wherein said antigenic peptide is derived from the target tissue of an autoimmune disease.

39. The compound of claim 31, wherein said antigenic peptide is derived from a cancer cell.

40. A compound comprising:
(a) two or more MHC-peptide complexes;
(b) a multivalent compound; and
(c) an antibody or a fragment thereof specific for a cell surface marker;
wherein said MHC-peptide complexes comprise either (i) an MHC class I α chain or fragment thereof and β₂-microglobulin or fragment thereof; or (ii) an MHC class II α chain or fragment thereof and an MHC class II β chain or fragment thereof; and an antigenic peptide bound in the MHC groove;
wherein at least one chain or fragment thereof of said MHC-peptide complexes are linked to said multivalent compound; and wherein said multivalent compound is linked to said antibody.

41. The compound of claim 40, wherein said MHC-peptide complex comprises an MHC class I α chain or fragment thereof and β₂-microglobulin or fragment thereof.

42. The compound of claim 40, wherein said MHC-peptide complex comprises an MHC class II α chain or fragment thereof and an MHC class II β chain or fragment thereof.

43. The compound of claim 40, wherein said cell surface marker is a cell surface marker of a professional antigen presenting cell.

44. The compound of claim 43, wherein said professional antigen presenting cell is a dendritic cell.

45. The compound of claim 44, wherein said cell surface marker is selected from the group consisting of CD83, CMRF-44, CMRF-56 and DEC-205.

46. The compound of claim 40, wherein said cell surface marker is a cell surface marker of a tumor cell.

47. The compound of claim 40, wherein said cell surface marker is a cell surface marker of an epithelial cell.

48. The compound of claim 40, wherein said cell surface marker is a cell surface marker of a fibroblast.

49. The compound of claim 40, wherein said cell surface marker is a cell surface marker of a T cell.

50. The compound of claim 49, wherein said cell surface marker is selected from the group consisting of CD28, CTLA-4 and CD25.

51. The compound of claim 40, wherein said antigenic peptide is derived from a cancer cell.

52. The compound of claim 40, wherein said antigenic peptide is derived from an infectious agent or from infected cells.

53. The compound of claim 40, wherein said antigenic peptide is derived from the target tissue of an autoimmune disease.

54. The compound of claim 46, wherein said antigenic peptide is derived from a cancer cell.

55. The compound of claim 40, further comprising a cytokine.

56. The compound of claim 40, wherein said multivalent compound is avidin.

57. The compound of claim 40, wherein said multivalent compound is selected from the group consisting of streptavidin and chicken avidin.

58. The compound of claim 40, wherein said multivalent compound is a modified GCN4-zipper motif.

59. A polynucleotide encoding a compound comprising:
(a) one or more MHC molecules; and
(b) an antibody or fragment thereof specific for a cell surface marker;
wherein said MHC molecules comprise an MHC class I α chain or fragment thereof and a β₂-microglobulin molecule or fragment thereof;
and wherein said MHC molecules are linked to the carboxyl terminus of said antibody or fragment thereof.

60. A polynucleotide encoding a compound comprising:
(a) one or more MHC molecules; and
(b) an antibody or fragment thereof specific for a cell surface marker;
wherein said MHC molecules comprise an MHC class I α chain or fragment thereof and a β₂-microglobulin molecule or fragment thereof;
and wherein said a chain of said MHC molecules are linked to the carboxyl terminus of said antibody or fragment thereof.

61. A polynucleotide encoding a compound comprising:
(a) one or more MHC molecules; and
(b) an antibody or fragment thereof specific for a cell surface marker;
wherein said MHC molecules comprise an MHC class II α chain or fragment thereof and an MHC class II β or fragment thereof;
and wherein at least one chain or fragment thereof of said MHC molecules are linked to the carboxyl terminus of said antibody or fragment thereof.

62. A method of immunizing an animal, comprising administering to said animal a compound comprising:
(a) one or more MHC-peptide complexes; and
(b) an antibody or a fragment thereof specific for a cell surface marker;
wherein said MHC-peptide complexes comprise an MHC class I α chain or fragment thereof, a β₂-microglobulin molecule or fragment thereof, and an antigenic peptide bound in the MHC groove; and
wherein said MHC-peptide complexes are linked to the carboxyl terminus of said antibody or fragment thereof.

63. The method of claim 62, wherein said cell surface marker is a cell surface marker of a professional antigen presenting cell.

64. The method of claim 63, wherein said professional antigen presenting cell is a dendritic cell.

65. The method of claim 64, wherein said cell surface marker is selected from the group consisting of CD83, CMRF-44, CMRF-56 and DEC-205.

66. The method of claim 62, wherein said cell surface marker is a cell surface marker of a tumor cell.

67. The method of claim 62, wherein said cell surface marker is a cell surface marker of an epithelial cell.

68. The method of claim 62, wherein said cell surface marker is a cell surface marker of a fibroblast.

69. The method of claim 62, wherein said cell surface marker is a cell surface marker of a T cell.

70. The method of claim 69, wherein said cell surface marker is selected from the group consisting of CD28, CTLA-4 and CD25.

71. The method of claim 62, wherein said antigenic peptide is derived from a cancer cell.

72. The method of claim 62, wherein said antigenic peptide is derived from an infectious agent or from infected cells.

73. The method of claim 62, wherein said antigenic peptide is derived from the target tissue of an autoimmune disease.

74. The method of claim 66, wherein said antigenic peptide is derived from a cancer cell.

75. A method of immunizing an animal, comprising administering to said animal a compound comprising:
(a) one or more MHC-peptide complexes; and
(b) an antibody or a fragment thereof specific for a cell surface marker;
wherein said MHC-peptide complexes comprise an MHC class I α chain or fragment thereof, a β₂-microglobulin molecule or fragment thereof, and an antigenic peptide bound in the MHC groove; and
wherein said MHC class I α chain or fragment thereof of said MHC-peptide complexes are linked to the carboxyl terminus of said antibody or fragment thereof.

76. The method of claim 75, wherein said cell surface marker is a cell surface marker of a professional antigen presenting cell.

77. The method of claim 76, wherein said professional antigen presenting cell is a dendritic cell.

78. The method of claim 77, wherein said cell surface marker is selected from the group consisting of CD83, CMRF-44, CMRF-56 and DEC-205.

79. The method of claim 75, wherein said cell surface marker is a cell surface marker of a tumor cell.

80. The method of claim 75, wherein said cell surface marker is a cell surface marker of an epithelial cell.

81. The method of claim 75, wherein said cell surface marker is a cell surface marker of a fibroblast.

82. The method of claim 75, wherein said cell surface marker is a cell surface marker of a T cell.

83. The method of claim 82, wherein said cell surface marker is selected from the group consisting of CD28, CTLA-4 and CD25.

84. The method of claim 75, wherein said antigenic peptide is derived from a cancer cell.

85. The method of claim 75, wherein said antigenic peptide is derived from an infectious agent or from infected cells.

86. The method of claim 75, wherein said antigenic peptide is derived from the target tissue of an autoimmune disease.

87. The method of claim 79, wherein said antigenic peptide is derived from a cancer cell.

88. A method of immunizing an animal, comprising administering to said animal a compound comprising:
(a) one or more MHC-peptide complexes; and
(b) an antibody or fragment thereof specific for a cell surface marker;
wherein said MHC-peptide complexes comprise an MHC class II α chain or fragment thereof, an MHC class II β chain or fragment thereof, and an antigenic peptide bound in the MHC groove; and
wherein at least one chain or fragment thereof of said MHC-peptide complexes are linked to the carboxyl terminus of said antibody or fragment thereof.

89. The method of claim 88, wherein said cell surface marker is a cell surface marker of a professional antigen presenting cell.

90. The method of claim 89, wherein said professional antigen presenting cell is a dendritic cell.

91. The method of claim 90, wherein said cell surface marker is selected from the group consisting of CD83, CMRF-44, CMRF-56 and DEC-205.

92. The method of claim 88, wherein said cell surface marker is a cell surface marker of a tumor cell.

93. The method of claim 88, wherein said cell surface marker is a cell surface marker of an epithelial cell.

94. The method of claim 88, wherein said cell surface marker is a cell surface marker of a fibroblast.

95. The method of claim 88, wherein said cell surface marker is a cell surface marker of a T cell.

96. The method of claim 95, wherein said cell surface marker is selected from the group consisting of CD28, CTLA-4 and CD25.

97. The method of claim 88, wherein said antigenic peptide is derived from a cancer cell.

98. The method of claim 88, wherein said antigenic peptide is derived from an infectious agent or from infected cells.

99. The method of claim 88, wherein said antigenic peptide is derived from the target tissue of an autoimmune disease.

100. The method of claim 92, wherein said antigenic peptide is derived from a cancer cell.

101. A method of immunizing an animal, comprising administering to said animal a compound comprising:
(a) two or more MHC-peptide complexes;
(b) a multivalent compound; and
(c) an antibody or a fragment thereof specific for a cell surface marker;
wherein said MHC-peptide complexes comprise either (i) an MHC class I α chain or fragment thereof and β₂-microglobulin or fragment thereof, or (ii) an MHC class II α chain or fragment thereof and an MHC class II β chain or fragment thereof; and an antigenic peptide bound in the MHC groove;
wherein at least one chain or fragment thereof of said MHC-peptide complexes are linked to said multivalent compound; and wherein said multivalent compound is linked to said antibody.

102. The method of claim 101, wherein said MHC-peptide complex comprises an MHC class I α chain or fragment thereof and β₂-microglobulin or fragment thereof.

103. The method of claim 101, wherein said MHC-peptide complex comprises an MHC class II α chain or fragment thereof and an MHC class II β chain or fragment thereof.

104. The method of claim 101, wherein said cell surface marker is a cell surface marker of a professional antigen presenting cell.

105. The method of claim 104, wherein said professional antigen presenting cell is a dendritic cell.

106. The method of claim 105, wherein said cell surface marker is selected from the group consisting of CD83, CMRF-44, CMRF-56 and DEC-205.

107. The method of claim 101, wherein said cell surface marker is a cell surface marker of a tumor cell.

108. The method of claim 101, wherein said cell surface marker is a cell surface marker of an epithelial cell.

109. The method of claim 101, wherein said cell surface marker is a cell surface marker of a fibroblast.

110. The method of claim 101, wherein said cell surface marker is a cell surface marker of a T cell.

111. The method of claim 110, wherein said cell surface marker is selected from the group consisting of CD28, CTLA-4 and CD25.

112. The method of claim 101, wherein said antigenic peptide is derived from a cancer cell.

113. The method of claim 1101, wherein said antigenic peptide is derived from an infectious agent or from infected cells.

114. The method of claim 101, wherein said antigenic peptide is derived from the target tissue of an autoimmune disease.

115. The method of claim 107, wherein said antigenic peptide is derived from a cancer cell.

116. The method of claim 101, further comprising administering a cytokine to said mammal.

117. The compound of claim 101, wherein said multivalent compound is avidin.

118. The compound of claim 101, wherein said multivalent compound is selected from the group consisting of streptavidin and chicken avidin.

119. The compound of claim 101, wherein said multivalent compound is a modified GCN4-zipper motif.
